(19) United States
(12) Patent Application Publication
(10) Pub. No.: US 2012/0232066 A1
(43) Pub. Date: Sep. 13, 2012

(54) COMPOSITIONS FOR REDUCING RISK OF ADVERSE EVENTS CAUSED BY DRUG-DRUG INTERACTIONS

(76) Inventors: Thomas E. JENKINS, Half Moon Bay, CA (US); Alex Gregory Sturmer, Danville, CA (US)

(21) Appl. No.: 13/415,761

(22) Filed: Mar. 8, 2012

Related U.S. Application Data
(60) Provisional application No. 61/451,041, filed on Mar. 9, 2011.

(51) Int. Cl. A61K 31/5517; A61P 21/02; A61P 25/00

(52) U.S. Cl. 514/220

(57) ABSTRACT

The present disclosure provides a composition comprising a GABA_{A} agonist and a GI enzyme inhibitor. The present disclosure also provides a composition comprising (a) a GI enzyme inhibitor and (b) a first drug that interacts with a second drug to produce an adverse effect when the second drug is co-ingested as a GI enzyme-cleavable prodrug with the first drug. Such an interaction can be additive or synergistic.

### CROSS REFERENCE TO RELATED APPLICATIONS

This application claims the benefit of U.S. Provisional Application No. 61/451,041 filed Mar. 9, 2011, the entirety of which is incorporated herein by reference.

### BACKGROUND OF THE INVENTION

Drugs are rarely used singularly as a result of diversification of medicine. In many cases, more than one drug is co-ingested simultaneously. In certain cases, such drugs can have adverse events due to drug-drug interactions. There is a need for compositions that reduce the risk of serious adverse events caused by such drug-drug interactions.

### BRIEF SUMMARY OF THE INVENTION

The present disclosure provides a composition comprising a GABA_{A} agonist and a GI enzyme inhibitor. In certain embodiments, the GABA_{A} agonist is a benzodiazepine. In certain embodiments, the GI enzyme inhibitor is a trypsin inhibitor.

The present disclosure also provides a composition comprising (a) a GI enzyme inhibitor and (b) a first drug that interacts with a second drug to produce an adverse effect when the second drug is co-ingested as a GI enzyme-cleavable prodrug with the first drug. Such an interaction can be additive or synergistic.

The first drug is a drug that can cause an adverse effect when it is co-ingested with a second drug. Such an adverse effect is often due to the two drugs interacting additively or synergistically to produce an adverse drug-drug interaction. In certain embodiments, the first drug is selected from a GABA_{A} agonist, a drug that interacts with an adrenergic receptor, an NMDA receptor antagonist, a monoamine oxidase inhibitor (MAOI), a central nervous system (CNS) depressant, and a drug that causes serotonin syndrome. In certain embodiments, the first drug is a muscle relaxant.

In certain embodiments, the present disclosure provides a composition that comprises a GABA_{A} agonist and a GI enzyme inhibitor.

In certain embodiments, the present disclosure provides a composition that comprises a CNS depressant and a GI enzyme inhibitor.

In certain embodiments, the second drug is a drug that is susceptible to misuse, abuse, or overdose, such as an opioid, amphetamine, or an amphetamine analog. The second drug is administered as a GI enzyme-cleavable prodrug. A “GI enzyme-cleavable prodrug” is a prodrug that comprises a promoiety comprising a GI enzyme-cleavable moiety. A GI enzyme-cleavable moiety has a site that is susceptible to cleavage by a GI enzyme.

The GI enzyme inhibitor of the composition can attenuate the action of GI enzyme(s). The GI enzyme inhibitor of the composition can interact with the GI enzyme(s) that mediates the controlled release of the second drug from the prodrug so as to attenuate enzymatic cleavage of the prodrug, thereby attenuating release of the drug.

### BRIEF DESCRIPTION OF THE FIGURES

FIG. 1 is a graph that compares mean blood concentrations over time of hydromorphone (HM) following PO administration to rats of prodrug Compound PC-1 alone and prodrug Compound PC-1 with various amounts of trypsin inhibitor from Glycine max (soybean) (SBTI).

FIG. 2 compares mean plasma concentrations over time of hydromorphone release following PO administration of prodrug Compound PC-5 with increasing amounts of co-dosed trypsin inhibitor Compound 109 to rats.

FIG. 3A and FIG. 3B compare mean plasma concentrations over time of hydromorphone release following PO administration of a single dose unit and of multiple dose units of a composition comprising prodrug Compound PC-5 and trypsin inhibitor Compound 109 to rats.

FIG. 4 compares mean plasma concentrations over time of oxycodone release following PO administration of prodrug Compound KC-2 with increasing amounts of co-dosed trypsin inhibitor Compound 109 to rats.

FIG. 5 compares mean plasma concentrations over time of oxycodone release following PO administration of prodrug Compound KC-3 with increasing amounts of co-dosed trypsin inhibitor Compound 109 to rats.

FIG. 6A and FIG. 6B compare mean plasma concentrations over time of oxycodone release following PO administration to rats of two doses of prodrug Compound KC-7, each co-dosed with increasing amounts of trypsin inhibitor Compound 109.

FIG. 7A compares mean plasma concentrations over time of oxycodone release following PO administration to rats of single and multiple doses of prodrug Compound KC-8 in the absence of trypsin inhibitor. FIG. 7B compares mean plasma concentrations over time of oxycodone release following PO administration to rats of single and multiple dose units comprising prodrug Compound KC-8 and trypsin inhibitor Compound 109.

FIG. 8 compares mean plasma concentrations over time of oxycodone release following PO administration to rats of prodrug Compound KC-17 co-dosed with increasing amounts of trypsin inhibitor Compound 109.

FIG. 9 provides a graph of mean plasma concentrations over time of amphetamine release following PO administration of prodrug Compound AM-1 with or without a co-dose of trypsin inhibitor according to embodiments of the present disclosure.

FIG. 10 shows a graph of mean plasma concentrations over time of amphetamine release following PO administration of prodrug Compound AM-2 with or without a co-dose of trypsin inhibitor according to embodiments of the present disclosure.

FIG. 11 compares mean plasma concentrations over time of hydromorphone following PO administration to dogs of (a) Compound PC-5, (b) co-administration of Compound PC-5 with Alprazolam XR, and (c) co-administration of Compound PC-5 and Compound 109 with Alprazolam XR.

FIG. 12 compares mean plasma concentrations over time of Alprazolam XR following PO administration to dogs of (a) Alprazolam XR, (b) co-administration of Alprazolam XR with Compound 109, (c) co-administration of Alprazolam XR with Compound PC-5 and, (d) co-administration of Alprazolam XR with Compound PC-5 and Compound 109.

### DETAILED DESCRIPTION OF THE INVENTION

### Definitions

The following terms have the following meaning unless otherwise indicated. Any undefined terms have their art recognized meanings.

“Alkyl” by itself or as part of another substituent refers to a saturated branched or straight-chain monovalent hydrocarbon radical derived by the removal of one hydrogen atom from a single carbon atom of a parent alkane. Typical alkyl groups include, but are not limited to, methyl; ethyl, propyls such as propan-1-yl or propan-2-yl; and butyls such as butan-1-yl, butan-2-yl, 2-methyl-propan-1-yl or 2-methyl-propan-2-yl. In some embodiments, an alkyl group comprises from 1 to 20 carbon atoms. In other embodiments, an alkyl group comprises from 1 to 10 carbon atoms. In still other embodiments, an alkyl group comprises from 1 to 6 carbon atoms, such as from 1 to 4 carbon atoms.

“Alkanyl” by itself or as part of another substituent refers to a saturated branched, straight-chain or cyclic alkyl radical derived by the removal of one hydrogen atom from a single carbon atom of an alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkylene” refers to a branched or unbranched saturated hydrocarbon chain, usually having from 1 to 40 carbon atoms, more usually 1 to 10 carbon atoms and even more usually 1 to 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

“Alkenyl” by itself or as part of another substituent refers to an unsaturated branched, straight-chain or cyclic alkyl radical having at least one carbon-carbon double bond derived by the removal of one hydrogen atom from a single carbon atom of an alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl(allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent refers to an unsaturated branched, straight-chain or cyclic alkyl radical having at least one carbon-carbon triple bond derived by the removal of one hydrogen atom from a single carbon atom of an alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Acyl” by itself or as part of another substituent refers to a radical —C(O)R³⁰, where R³⁰ is hydrogen, alkyl, cycloalkyl, cycloheteroalkyl, aryl, arylalkyl, heteroalkyl, heteroaryl, heteroarylalkyl as defined herein and substituted versions thereof. Representative examples include, but are not limited to formyl, acetyl, cyclohexylcarbonyl, cyclohexylmethylcarbonyl, benzoyl, benzylcarbonyl, piperonyl, succinyl, and malonyl, and the like.

“Acylamino” refers to the groups —NR²⁰C(O)alkyl, —NR²⁰C(O)substituted alkyl, N R²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

“Aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Alkoxy” by itself or as part of another substituent refers to a radical —OR³¹ where R³¹ represents an alkyl or cycloalkyl group as defined herein. Representative examples include, but are not limited to, methoxy, ethoxy, propoxy, butoxy, cyclohexyloxy and the like.

“Alkoxycarbonyl” by itself or as part of another substituent refers to a radical —C(O)OR³¹ where R³¹ represents an alkyl or cycloalkyl group as defined herein. Representative examples include, but are not limited to, methoxycarbonyl, ethoxycarbonyl, propoxycarbonyl, butoxycarbonyl, cyclohexyloxycarbonyl and the like.

“Aryl” by itself or as part of another substituent refers to a monovalent aromatic hydrocarbon radical derived by the removal of one hydrogen atom from a single carbon atom of an aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In certain embodiments, an aryl group comprises from 6 to 20 carbon atoms. In certain embodiments, an aryl group comprises from 6 to 12 carbon atoms. Examples of an aryl group are phenyl and naphthyl.

“Arylalkyl” by itself or as part of another substituent refers to an acyclic alkyl radical in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In certain embodiments, an arylalkyl group is (C₇-C₃₀) arylalkyl, e.g., the alkanyl, alkenyl or alkynyl moiety of the arylalkyl group is (C₁-C₁₀) and the aryl moiety is (C₆-C₂₀). In certain embodiments, an arylalkyl group is (C₇-C₂₀) arylalkyl, e.g., the alkanyl, alkenyl or alkynyl moiety of the arylalkyl group is (C₁-C₈) and the aryl moiety is (C₆-C₁₂).

“Arylaryl” by itself or as part of another substituent, refers to a monovalent hydrocarbon group derived by the removal of one hydrogen atom from a single carbon atom of a ring system in which two or more identical or non-identical aromatic ring systems are joined directly together by a single bond, where the number of such direct ring junctions is one less than the number of aromatic ring systems involved. Typical arylaryl groups include, but are not limited to, biphenyl, triphenyl, phenyl-napthyl, binaphthyl, biphenyl-napthyl, and the like. When the number of carbon atoms in an arylaryl group is specified, the numbers refer to the carbon atoms comprising each aromatic ring. For example, (C₅-C₁₄) arylaryl is an arylaryl group in which each aromatic ring comprises from 5 to 14 carbons, e.g., biphenyl, triphenyl, binaphthyl, phenylnapthyl, etc. In certain embodiments, each aromatic ring system of an arylaryl group is independently a (C₅-C₁₄) aromatic. In certain embodiments, each aromatic ring system of an arylaryl group is independently a (C₅-C₁₀) aromatic. In certain embodiments, each aromatic ring system is identical, e.g., biphenyl, triphenyl, binaphthyl, trinaphthyl, etc.

“Carboxyl,” “carboxy” or “carboxylate” refers to —CO₂H or salts thereof.

“Cyano” or “nitrile” refers to the group —CN.

“Cycloalkyl” by itself or as part of another substituent refers to a saturated or unsaturated cyclic alkyl radical. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane and the like. In certain embodiments, the cycloalkyl group is (C₃-C₁₀) cycloalkyl. In certain embodiments, the cycloalkyl group is (C₃-C₇) cycloalkyl.

“Cycloheteroalkyl” or “heterocyclyl” by itself or as part of another substituent, refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidine, quinuclidine and the like.

“Heteroalkyl, Heteroalkanyl, Heteroalkenyl and Heteroalkynyl” by themselves or as part of another substituent refer to alkyl, alkanyl, alkenyl and alkynyl groups, respectively, in which one or more of the carbon atoms (and any associated hydrogen atoms) are independently replaced with the same or different heteroatomic groups. Typical heteroatomic groups which can be included in these groups include, but are not limited to, —O—, —S—, —S—S—, —O—S—, —NR³⁷R³⁸—, ═N—N═, —N═N—, —N═N—NR³⁹R⁴⁰, —PR⁴¹—, —P(O)₂—, —POR⁴²—, —O—P(O)₂—, —S—O—, —S—(O)—, —SO₂—, —SnR⁴³R⁴⁴— and the like, where R³⁷, R³⁸, R³⁹, R⁴⁰, R⁴¹, R⁴², R⁴³ and R⁴⁴ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent, refers to a monovalent heteroaromatic radical derived by the removal of one hydrogen atom from a single atom of a heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, benzodioxole and the like. In certain embodiments, the heteroaryl group is from 5-20 membered heteroaryl. In certain embodiments, the heteroaryl group is from 5-10 membered heteroaryl. In certain embodiments, heteroaryl groups are those derived from thiophene, pyrrole, benzothiophene, benzofuran, indole, pyridine, quinoline, imidazole, oxazole and pyrazine.

“Heteroarylalkyl” by itself or as part of another substituent, refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heteroarylalkynyl is used. In certain embodiments, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20-membered heteroaryl. In certain embodiments, the heteroarylalkyl group is 6-20 membered heteroarylalkyl, e.g., the alkanyl, alkenyl or alkynyl moiety of the heteroarylalkyl is 1-8 membered and the heteroaryl moiety is a 5-12-membered heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 15 ring atoms, including 1 to 4 hetero atoms. These hetero atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

“Aromatic Ring System” by itself or as part of another substituent, refers to an unsaturated cyclic or polycyclic ring system having a conjugated π electron system. Specifically included within the definition of “aromatic ring system” are fused ring systems in which one or more of the rings are aromatic and one or more of the rings are saturated or unsaturated, such as, for example, fluorene, indane, indene, phenalene, etc. Typical aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Heteroaromatic Ring System” by itself or as part of another substituent, refers to an aromatic ring system in which one or more carbon atoms (and any associated hydrogen atoms) are independently replaced with the same or different heteroatom. Typical heteroatoms to replace the carbon atoms include, but are not limited to, N, P, O, S, Si, etc. Specifically included within the definition of “heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Typical heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Substituted” refers to a group in which one or more hydrogen atoms are independently replaced with the same or different substituent(s). Typical substituents include, but are not limited to, alkylenedioxy (such as methylenedioxy), -M, —R⁶⁰, —O⁻, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂O⁻, —S(O)₂OH, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)NR⁶⁰R⁶¹, C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹ and —C(NR⁶²)NR⁶⁰R⁶¹ where M is halogen; R⁶⁰, R⁶¹, R⁶² and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶⁴ and R⁶⁵ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶⁴ and R⁶⁵ together with the nitrogen atom to which they are bonded form a cycloheteroalkyl or substituted cycloheteroalkyl ring. In certain embodiments, substituents include -M, —R⁶⁰, ═O, —OR⁶⁰, —SR⁶⁰, S′, ═S, —NR⁶⁰R⁶¹, ═NR⁶⁰, —CF₃, —CN, —OCN, —SCN, —NO, —NO₂, ═N₂, —N₃, —S(O)₂R⁶⁰, —OS(O)₂O⁻, —OS(O)₂R⁶⁰, P(O)(O⁻)₂, —P(O)(OR⁶)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, C(O)O⁻, —NR⁶²C(O)NR⁶⁰R⁶¹. In certain embodiments, substituents include -M, —R⁶⁰, ═O, —OR⁶⁰, —SR⁶⁰, —NR⁶⁰R⁶¹, CF₃, —CN, —NO₂, —S(O)₂R⁶⁰, —P(O)(OR⁶⁰)(O⁻), —OP(O)(OR⁶⁰)(OR⁶¹), —C(O)R⁶⁰, —C(O)OR⁶⁰, —C(O)_{NR}⁶⁰R⁶¹, —C(O)O⁻. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(OR⁶⁰), —C(O)OR⁶⁰, —C(O)O⁻, where R⁶⁰, R⁶¹ and R⁶² are as defined above. For example, a substituted group may bear a methylenedioxy substituent or one, two, or three substituents selected from a halogen atom, a (1-4C)alkyl group and a (1-4C)alkoxy group.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups are limited to substituted aryl-(substituted aryl)-substituted aryl.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

“Dose unit” as used herein refers to a combination of a GI enzyme-cleavable prodrug (e.g., trypsin-cleavable prodrug) and a GI enzyme inhibitor (e.g., a trypsin inhibitor). A “single dose unit” is a single unit of a combination of a GI enzyme-cleavable prodrug (e.g., trypsin-cleavable prodrug) and a GI enzyme inhibitor (e.g., trypsin inhibitor), where the single dose unit provide a therapeutically effective amount of drug (i.e., a sufficient amount of drug to effect a therapeutic effect, e.g., a dose within the respective drug's therapeutic window, or therapeutic range). “Multiple dose units” or “multiples of a dose unit” or a “multiple of a dose unit” refers to at least two single dose units.

“Gastrointestinal enzyme” or “GI enzyme” refers to an enzyme located in the gastrointestinal (GI) tract, which encompasses the anatomical sites from mouth to anus. Trypsin is an example of a GI enzyme.

“Gastrointestinal enzyme-cleavable moiety” or “GI enzyme-cleavable moiety” refers to a group comprising a site susceptible to cleavage by a GI enzyme. For example, a “trypsin-cleavable moiety” refers to a group comprising a site susceptible to cleavage by trypsin.

“Gastrointestinal enzyme inhibitor” or “GI enzyme inhibitor” refers to any agent capable of inhibiting the action of a gastrointestinal enzyme on a substrate. The term also encompasses salts of gastrointestinal enzyme inhibitors. For example, a “trypsin inhibitor” refers to any agent capable of inhibiting the action of trypsin on a substrate.

“Patient” includes humans, and also other mammals, such as livestock, zoo animals, and companion animals, such as a cat, dog, or horse.

“Pharmaceutical composition” refers to at least one compound and can further comprise a pharmaceutically acceptable carrier, with which the compound is administered to a patient.

“Pharmaceutically acceptable carrier” refers to a diluent, adjuvant, excipient or vehicle with, or in which a compound is administered.

“Pharmaceutically acceptable salt” refers to a salt of a compound, which possesses the desired pharmacological activity of the compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonate, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Pharmacodynamic (PD) profile” refers to a profile of the efficacy of a drug in a patient (or subject or user), which is characterized by PD parameters. “PD parameters” include “drug Emax” (the maximum drug efficacy), “drug EC50” (the concentration of drug at 50% of the Emax) and side effects.

“PK parameter” refers to a measure of drug concentration in blood or plasma, such as: 1) “drug Cmax”, the maximum concentration of drug achieved in blood or plasma; 2) “drug Tmax”, the time elapsed following ingestion to achieve Cmax; and 3) “drug exposure”, the total concentration of drug present in blood or plasma over a selected period of time, which can be measured using the area under the curve (AUC) of a time course of drug release over a selected period of time (t). Modification of one or more PK parameters provides for a modified PK profile.

“PK profile” refers to a profile of drug concentration in blood or plasma. Such a profile can be a relationship of drug concentration over time (i.e., a “concentration-time PK profile”) or a relationship of drug concentration versus number of doses ingested (i.e., a “concentration-dose PK profile”). A PK profile is characterized by PK parameters.

“Preventing” or “prevention” or “prophylaxis” refers to a reduction in risk of occurrence of a condition, such as pain.

“Prodrug” refers to a derivative of an active agent that requires a transformation within the body to release the active agent. In certain embodiments, the transformation is an enzymatic transformation. In certain embodiments, the transformation is a cyclization transformation. In certain embodiments, the transformation is a combination of an enzymatic transformation and a cyclization transformation. Prodrugs are frequently, although not necessarily, pharmacologically inactive until converted to the active agent.

“Promoiety” refers to a form of protecting group that when used to mask a functional group within an active agent converts the active agent into a prodrug. Typically, the promoiety will be attached to the drug via bond(s) that are cleaved by enzymatic or non-enzymatic means in vivo.

“Solvate” as used herein refers to a complex or aggregate formed by one or more molecules of a solute, e.g. a prodrug or a pharmaceutically acceptable salt thereof, and one or more molecules of a solvent. Such solvates are typically crystalline solids having a substantially fixed molar ratio of solute and solvent. Representative solvents include by way of example, water, methanol, ethanol, isopropanol, acetic acid, and the like. When the solvent is water, the solvate formed is a hydrate.

“Therapeutically effective amount” means the amount of a compound (e.g., prodrug) that, when administered to a patient for preventing or treating a condition such as pain, is sufficient to effect such treatment. The “therapeutically effective amount” will vary depending on the compound, the condition and its severity and the age, weight, etc., of the patient.

“Treating” or “treatment” of any condition, such as pain, refers, in certain embodiments, to ameliorating the condition (i.e., arresting or reducing the development of the condition). In certain embodiments “treating” or “treatment” refers to ameliorating at least one physical parameter, which may not be discernible by the patient. In certain embodiments, “treating” or “treatment” refers to inhibiting the condition, either physically, (e.g., stabilization of a discernible symptom), physiologically, (e.g., stabilization of a physical parameter), or both. In certain embodiments, “treating” or “treatment” refers to delaying the onset of the condition.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It should be understood that as used herein, the term “a” entity or “an” entity refers to one or more of that entity. For example, a compound refers to one or more compounds. As such, the terms “a”, “an”, “one or more” and “at least one” can be used interchangeably. For example, a first drug refers to at least one first drug, and one or more first drugs. Similarly the terms “comprising”, “including” and “having” can be used interchangeably.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

The nomenclature used herein to name the subject compounds is illustrated in the Examples herein. In certain instances, this nomenclature is derived using the commercially-available AutoNom software (MDL, San Leandro, Calif.).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the chemical groups represented by the variables are specifically embraced by the present invention and are disclosed herein just as if each and every combination was individually and explicitly disclosed, to the extent that such combinations embrace compounds that are stable compounds (i.e., compounds that can be isolated, characterised, and tested for biological activity). In addition, all sub-combinations of the chemical groups listed in the embodiments describing such variables are also specifically embraced by the present invention and are disclosed herein just as if each and every such sub-combination of chemical groups was individually and explicitly disclosed herein.

### General Synthetic Procedures

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any of the means known in the art, including chromatographic means, such as high performance liquid chromatography (HPLC), preparative thin layer chromatography, flash column chromatography and ion exchange chromatography. Any suitable stationary phase can be used, including normal and reversed phases as well as ionic resins. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the compounds of the present disclosure, it may be necessary and/or desirable to protect sensitive or reactive groups on any of the molecules concerned. This can be achieved by means of conventional protecting groups as described in standard works, such as T. W. Greene and P. G. M. Wuts, “Protective Groups in Organic Synthesis”, Fourth edition, Wiley, New York 2006. The protecting groups can be removed at a convenient subsequent stage using methods known from the art.

The compounds described herein can contain one or more chiral centers and/or double bonds and therefore, can exist as stereoisomers, such as double-bond isomers (i.e., geometric isomers), enantiomers or diastereomers. Accordingly, all possible enantiomers and stereoisomers of the compounds including the stereoisomerically pure form (e.g., geometrically pure, enantiomerically pure or diastereomerically pure) and enantiomeric and stereoisomeric mixtures are included in the description of the compounds herein. Enantiomeric and stereoisomeric mixtures can be resolved into their component enantiomers or stereoisomers using separation techniques or chiral synthesis techniques well known to the skilled artisan. The compounds can also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that can be incorporated into the compounds disclosed herein include, but are not limited to, ²H, ³H, ¹¹C, ¹³C, ¹⁴C, ¹⁵N, ¹⁸O, ¹⁷O, etc. Compounds can exist in unsolvated forms as well as solvated forms, including hydrated forms. In general, compounds can be hydrated or solvated. Certain compounds can exist in multiple crystalline or amorphous forms. In general, all physical forms are equivalent for the uses contemplated herein and are intended to be within the scope of the present disclosure.

### Representative Embodiments

Reference will now be made in detail to various embodiments. It will be understood that the invention is not limited to these embodiments. To the contrary, it is intended to cover alternatives, modifications, and equivalents as may be included within the spirit and scope of the allowed claims.

The present disclosure provides a composition comprising a GABA_{A} agonist and a GI enzyme inhibitor. In certain embodiments, the GABA_{A} agonist is a benzodiazepine. In certain instances the GABA_{A} agonist is a drug that exerts a similar effect at a GABA_{A} receptor. In certain embodiments, the GI enzyme inhibitor is a trypsin inhibitor.

The present disclosure also provides a composition comprising (a) a GI enzyme inhibitor and (b) a first drug that interacts with a second drug to produce an adverse effect when the second drug is co-ingested as a prodrug with the first drug. Such an interaction can be additive or synergistic.

The first drug is a drug that causes an adverse effect when it is co-ingested with a second drug. Such an adverse effect is often due to the two drugs interacting additively or synergistically to produce an adverse drug-drug interaction.

In certain embodiments, the second drug is a drug that is susceptible to misuse, abuse, or overdose, such as an opioid, amphetamine, or an amphetamine analog. The second drug is administered as a GI enzyme-cleavable prodrug. A “GI enzyme-cleavable prodrug” is a prodrug that comprises a promoiety comprising a GI enzyme-cleavable moiety. A GI enzyme-cleavable moiety has a site that is susceptible to cleavage by a GI enzyme.

The GI enzyme inhibitor of the composition can attenuate the action of GI enzyme(s). The GI enzyme inhibitor of the composition can interact with the GI enzyme(s) that mediates the controlled release of the second drug from the prodrug so as to attenuate enzymatic cleavage of the prodrug, thereby attenuating release of the drug.

Examples of first drugs, GI enzyme inhibitors, and GI enzyme-cleavable prodrugs that release second drugs are described herein.

### First Drugs

The first drug interacts with a second drug to produce an adverse drug-drug interaction. That is, co-ingestion of the first drug and the second drug lead to an additive or synergistic pharmacodynamic effect, which can lead to adverse effects, even death. In certain embodiments, the first drug is selected from a GABA_{A} agonist, a drug that interacts with an adrenergic receptor, an NMDA receptor antagonist, a monoamine oxidase inhibitor (MAOI), a central nervous system (CNS) depressant, and a drug that causes serotonin syndrome. In certain embodiments, the first drug is a muscle relaxant.

In certain embodiments, the first drug is a GABA_{A} agonist. In certain embodiments, the first drug is selected from a drug that interacts with an adrenergic receptor, an NMDA receptor antagonist, a monoamine oxidase inhibitor (MAUI), a central nervous system (CNS) depressant, and a drug that causes serotonin syndrome.

GABA is an inhibitory neurotransmitter in the brain, which is known to affect mood stabilizing activity, anxiolytic activity and muscle relaxant activity, and is further known to be related to some central nervous system disorders and diseases. GABA_{A} agonists can stimulate or increase the action at the GABA receptor, producing typically sedative effects, and may also cause other effects such as anxiolytic and muscle relaxant effects.

Examples of GABA_{A} agonists include, but are not limited to, benzodiazepines, non-benzodiazepines, barbiturates, neuroactive steroids, methaqualone, progabide, and tiagabine.

Benzodiazepines enhance the effect of GABA, which results in sedative, hypnotic (sleep-inducing), anxiolytic (anti-anxiety), anticonvulsant, muscle relaxant and amnesic action. The structure of benzodiazepines includes a fusion of a benzene ring and a diazepine ring, as shown in the following structure:

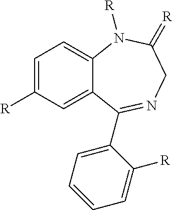

Examples of benzodiazepines include, but are not limited to, alprazolam, bretazenil, bromazepam, brotizolam, chlordiazepoxide, cinolazepam, clonazepam, cloxazolam, clorazepate, delorazepam, diazepam, estazolam, flunitrazepam, flurazepam, flutopazepam, halazepam, ketazolam, loprazolam, lorazepam, lormetazepam, midazolam, nimetazepam, nitrazepam, nordazepam, oxazepam, phenazepam, pinazepam, prazepam, premazepam, quazepam, temazepam, tetrazepam, clobazam, flumazenil, eszopiclone, zaleplon, zolpidem, and zopiclone.

Non-benzodiazepines, also called benzodiazepine-like drugs, are a class of psychoactive drugs whose pharmacological actions are similar to those of the benzodiazepines, but are structurally distant or unrelated to the benzodiazepines on a chemical level. They have side effects and benefits and risks similar to benzodiazepines. Subclasses of non-benzodiazepines include imidazopyridines, pyrazolopyrimidines, and cyclopyrrolones. Imidazopyridines have the following structure:

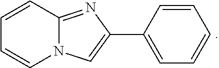

Examples of imidazopyridines include, but are not limited to, Zolpidem (AMBIEN), Alpidem, Saripidem, Necopidem, and DS-1. Pyrazolopyrimidines have the following structure:

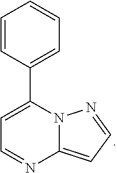

Examples of pyrazolopyrimidines include, but are not limited to, Zaleplon (SONATA), Fasiplon, Indiplon, Ocinaplon, Panadiplon, and Taniplon. Cyclopyrrolones have the following structure:

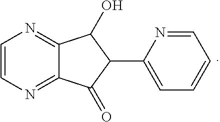

Examples of cyclopyrrolones include, but are not limited to, Eszopiclone (LUNESTA), Zopiclone (IMOVANE), Pagoclone, Pazinaclone, Suproclone, and Suriclone.

Barbiturates are drugs that act as central nervous system depressants and produce a wide spectrum of effects, from mild sedation to total anesthesia. Barbiturates are derivatives of barbituric acid:

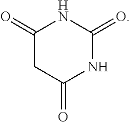

Examples of barbiturates include, but are not limited to, allobarbital, amobarbital, aprobarbital, alphenal, barbital, brallobarbital, and phenobarbital.

Neuroactive steroids (or neurosteroids) rapidly alter neuronal excitability through interaction with neurotransmitter-gated ion channels. Neurosteroids have a wide range of potential clinical applications from sedation to treatment of epilepsy and traumatic brain injury. Neuroactive steroids have a steroid core structure, as follows:

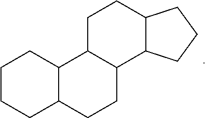

Examples of neuroactive steroids include, but are not limited to, alphaxolone, alphadolone, hydroxydione, and minoxolone.

Methaqualone is a sedative-hypnotic drug that is similar in effect to barbiturates, a general central nervous system depressant. Methaqualone is also known as Quaaludes, Sopors, Ludes or Mandrax. Methaqualone has the following structure:

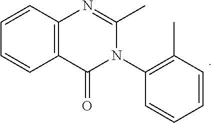

Progabide (GABRENE) is an analog and prodrug of gamma-aminobutyric acid used in the treatment of epilepsy. It has agonistic activity at both the GABA_{A} and GABA_{B} receptors. Progabide has the following structure:

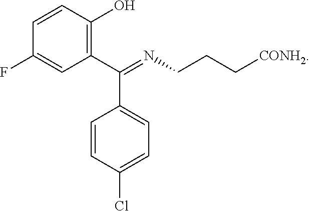

Tiagabine (GABITRIL) is an anti-convulsive medication. The medication is also used in the treatment of panic disorder, as are a few other anticonvulsants. Tiagabine has the following structure:

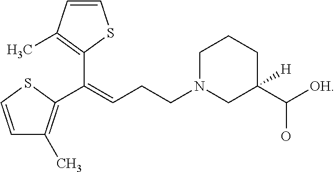

One embodiment is a drug that interacts with an adrenergic receptor, such as an alpha-adrenergic receptor or a beta-adrenergic receptor. One embodiment is a drug that antagonizes an alpha- or beta-adrenergic receptor. One embodiment is an alpha-blocker. One embodiment is a beta-blocker.

One embodiment is a NMDA receptor antagonist.

One embodiment is a monoamine oxidase inhibitor (MAOI). Co-ingestion of an MAOI and a drug susceptible to misuse, abuse or overdose, such as an opioid (e.g., tapentadol), amphetamine or amphetamine analog, can lead to adverse drug-drug interactions. Examples of MAOIs include, but are not limited to, furazolidone, isocarboxazid, linezolid, moclobemide, phenelzine, procarbazine, rasagiline, selegiline, and tranylcypromine.

One embodiment is a central nervous system (CNS) depressant. One embodiment is a drug that when co-ingested with an opioid leads to respiratory depression, or hypoventilation. One embodiment is a muscle relaxant. Other embodiments include, but are not limited to, certain antihistamines, drugs for high blood pressure, anti-psychotics, pain medicines, anti-seizure drugs, stimulants, and veratrum alkaloids.

One embodiment is a drug that causes drowsiness such as certain antihistamines (such as diphenhydramine), anti-anxiety drugs (such as diazepam), tricyclic antidepressants (such as amitriptyline), anti-seizure drugs (such as phenyloin), medicine for sleep (such as zolpidem), and muscle relaxants (such as cyclobenzaprine).

One embodiment is a drug that can cause serotonin syndrome, particularly when co-ingested with an opioid, such as hydrocodone, oxycodone, or tapentadol. Examples of such drugs include antidepressants, CNS stimulants, and 5-HT₁ agonists.

Examples of antidepressants that, alone or in combination with another drug (such as an opioid), can lead to serotonin syndrome include, but are not limited to, monoamine oxidase inhibitors (MAOIs), TCAs, SSRIs (such as citalopram, paroxetine), SNRIs (such as duloxetine, venlafaxine), bupropion, nefazodone, and trazodone. Another example is St. John's wort.

Examples of CNS stimulants that, alone or in combination with another drug (such as an opioid), can lead to serotonin syndrome include, but are not limited to, phentermine, diethylpropion, amphetamine, sibutramine, methylphenidate, methamphetamine, and cocaine.

Examples of 5-HT₁ agonists that, alone or in combination with another drug (such as an opioid), can lead to serotonin syndrome include, but are not limited to, triptans (such as eletriptan, sumatriptan).

Other examples of a drug that, alone or in combination with another drug (such as an opioid), can lead to serotonin syndrome include, but are not limited to, tryptophan, L-Dopa, valproate, buspirone, lithium, linezolid, dextromethorphan, 5-hydroxytryptophan, chlorpheniramine, risperidone, olanzapine, ondansetron, granisetron, metoclopramide, and ritonavir.

### Enzyme Inhibitors

The GI enzyme inhibitor of the composition can attenuate the action of GI enzyme(s). The GI enzyme inhibitor of the composition can interact with the GI enzyme(s) that mediates the controlled release of the second drug from the prodrug so as to attenuate enzymatic cleavage of the prodrug.

The enzyme capable of cleaving the enzymatically-cleavable moiety of a prodrug can be a peptidase, also called a protease. In certain embodiments, the enzyme is an enzyme located in the gastrointestinal (GI) tract, i.e., a gastrointestinal enzyme, or a GI enzyme. The enzyme can be a digestive enzyme such as a gastric, intestinal, pancreatic or brush border enzyme or enzyme of GI microbial flora, such as those involved in peptide hydrolysis. Examples include a pepsin, such as pepsin A or pepsin B; a trypsin; a chymotrypsin; an elastase; a carboxypeptidase, such as carboxypeptidase A or carboxypeptidase B; an aminopeptidase (such as aminopeptidase N or aminopeptidase A; an endopeptidase; an exopeptidase; a dipeptidylaminopeptidase such as dipeptidylaminopeptidase IV; a dipeptidase; a tripeptidase; or an enteropeptidase. In certain embodiments, the enzyme is a cytoplasmic protease located on or in the GI brush border. In certain embodiments, the enzyme is trypsin. Accordingly, in certain embodiments, the corresponding composition is administered orally to the patient.

The disclosure provides for a composition comprising a GI enzyme inhibitor. Such an inhibitor can inhibit at least one of any of the GI enzymes disclosed herein. An example of a GI enzyme inhibitor is a protease inhibitor, such as a trypsin inhibitor.

As used herein, the term “GI enzyme inhibitor” refers to any agent capable of inhibiting the action of a GI enzyme on a substrate. The ability of an agent to inhibit a GI enzyme can be measured using assays well known in the art.

In certain embodiments, the GI enzyme capable of cleaving the enzymatically-cleavable moiety may be a protease—the enzymatically-cleavable moiety being linked to the nucleophilic nitrogen through an amide (e.g. a peptide: —NHC(O)—) bond. The disclosure provides for inhibitors of proteases.

Proteases can be classified as exopeptidases or endopeptidases. Examples of exopeptidases include aminopeptidase and carboxypeptidase (A, B, or Y). Examples of endopeptidases include trypsin, chymotrypsin, elastase, pepsin, and papain. The disclosure provides for inhibitors of exopeptidase and endopeptidase.

In some embodiments, the enzyme is a digestive enzyme of a protein. The disclosure provides for inhibitors of digestive enzymes. A gastric phase involves stomach enzymes, such as pepsin. An intestinal phase involves enzymes in the small intestine duodenum, such as trypsin, chymotrypsin, elastase, carboxypeptidase A, and carboxypeptidase B. An intestinal brush border phase involves enzymes in the small intestinal brush border, such as aminopeptidase N, aminopeptidase A, endopeptidases, dipeptidases, dipeptidylaminopeptidase, and dipeptidylaminopeptidase IV. An intestinal intracellular phase involves intracellular peptidases, such as dipeptidases (i.e. iminopeptidase) and aminopeptidase.

In certain embodiments, the enzyme inhibitor in the disclosed compositions is a peptidase inhibitor or protease inhibitor. In certain embodiments, the enzyme is a digestive enzyme such as a gastric, pancreatic or brush border enzyme, such as those involved in peptide hydrolysis. Examples include pepsin, trypsin, chymotrypsin, colipase, elastase, aminopeptidase N, aminopeptidase A, dipeptidylaminopeptidase IV, tripeptidase or enteropeptidase.

Proteases can be inhibited by naturally occurring peptide or protein inhibitors, or by small molecule naturally occurring or synthetic inhibitors. Examples of protein or peptide inhibitors that are protease inhibitors include, but are not limited to, α1-antitrypsin from human plasma, aprotinin, trypsin inhibitor from soybean (SBTI), Bowman-Birk Inhibitor from soybean (BBSI), trypsin inhibitor from egg white (ovomucoid), chromostatin, and potato-derived carboxypeptidase inhibitor. Examples of small molecule irreversible inhibitors that are protease inhibitors include, but are not limited to, TPCK (1-chloro-3-tosylamido-4-phenyl-2-butanone), TLCK (1-chloro-3-tosylamido-7-amino-2-heptone), and PMSF (phenylmethyl sulfonyl floride). Examples of small molecule irreversible inhibitors that are protease inhibitors include, but are not limited to benzamidine, apixaban, camostat, 3,4-dichloroisocoumarin, ε-aminocaprionic acid, amastatin, lysianadioic acid, 1,10-phenanthroline, cysteamine, and bestatin. Other examples of small molecule inhibitors are Compound 101, Compound 102, Compound 103, Compound 104, Compound 105, Compound 106, Compound 107, Compound 108, Compound 109 and Compound 110.

The following table shows examples of gastrointestinal (GI) proteases, examples of their corresponding substrates, and examples of corresponding inhibitors.

Table of Examples of GI Proteases and
Corresponding Substrates and Inhibitors

| GI Protease | Substrates | Inhibitors |
|---|---|---|
| Trypsin | Arg, Lys, | TLCK, Benzamidine, |
| | positively | Apixaban, Bowman Birk |
| | charged residues | |
| Chymotrypsin | Phe, Tyr, Trp, | ε-Aminocaprionic |
| | bulky | TPCK |
| | hydrophobic | Bowman-Birk |
| | residues | |
| Pepsin | Leu, Phe, Trp, | Pepstatin, PMSF |
| | Tyr | |
| Carboxypeptidase B | Arg, Lys | Potato-derived inhibitor, |
| | | Lysianadioic acid |
| Carboxypeptidase A | not Arg, Lys | Potato-derived inhibitor, 1,10- |
| | | phenanthroline |
| Elastase | Ala, Gly, Ser, | α1-antitrypsin, |
| | small neutral | 3,4-dichlorocoumarin |
| | residues | |
| Aminopeptidase | All free N- | Bestatin, Amastatin |
| | terminal AA | |

### Trypsin Inhibitors

As used herein, the term “trypsin inhibitor” refers to any agent capable of inhibiting the action of trypsin on a substrate. The term “trypsin inhibitor” also encompasses salts of trypsin inhibitors. The ability of an agent to inhibit trypsin can be measured using assays well known in the art. For example, in a typical assay, one unit corresponds to the amount of inhibitor that reduces the trypsin activity by one benzoyl-L-arginine ethyl ester unit (BAEE-U). One BAEE-U is the amount of enzyme that increases the absorbance at 253 nm by 0.001 per minute at pH 7.6 and 25° C. See, for example, K. Ozawa, M. Laskowski, 1966, J. Biol. Chem. 241, 3955 and Y. Birk, 1976, Meth. Enzymol. 45, 700. In certain instances, a trypsin inhibitor can interact with an active site of trypsin, such as the S1 pocket and the S3/4 pocket. The S1 pocket has an aspartate residue which has affinity for positively charged moiety. The S3/4 pocket is a hydrophobic pocket. The disclosure provides for specific trypsin inhibitors and non-specific serine protease inhibitors.

There are many trypsin inhibitors known in the art, both those specific to trypsin and those that inhibit trypsin and other proteases such as chymotrypsin. The disclosure provides for trypsin inhibitors that are proteins, peptides, and small molecules. The disclosure provides for trypsin inhibitors that are irreversible inhibitors or reversible inhibitors. The disclosure provides for trypsin inhibitors that are competitive inhibitors, non-competitive inhibitors, or uncompetitive inhibitors. The disclosure provides for natural, synthetic or semi-synthetic trypsin inhibitors.

Trypsin inhibitors can be derived from a variety of animal or vegetable sources: for example, soybean, corn, lima and other beans, squash, sunflower, bovine and other animal pancreas and lung, chicken and turkey egg white, soy-based infant formula, and mammalian blood. Trypsin inhibitors can also be of microbial origin: for example, antipain; see, for example, H. Umezawa, 1976, Meth. Enzymol. 45, 678.

In one embodiment, the trypsin inhibitor is derived from soybean. Trypsin inhibitors derived from soybean (Glycine max) are readily available and are considered to be safe for human consumption. They include, but are not limited to, SBTI, which inhibits trypsin, and Bowman-Birk inhibitor, which inhibits trypsin and chymotrypsin. Such trypsin inhibitors are available, for example from Sigma-Aldrich, St. Louis, Mo., USA.

A trypsin inhibitor can be an arginine mimic or lysine mimic, either natural or synthetic compound. In certain embodiments, the trypsin inhibitor is an arginine mimic or a lysine mimic, wherein the arginine mimic or lysine mimic is a synthetic compound. As used herein, an arginine mimic or lysine mimic can include a compound capable of binding to the P¹ pocket of trypsin and/or interfering with trypsin active site function. The arginine or lysine mimic can be a cleavable or non-cleavable moiety.

Examples of trypsin inhibitors, which are arginine mimics and/or lysine mimics, include, but not limited to, arylguanidine, benzamidine, 3,4-dichloroisocoumarin, diisopropylfluorophosphate, gabexate mesylate, and phenylmethanesulfonyl fluoride, or substituted versions or analogs thereof. In certain embodiments, trypsin inhibitors comprise a covalently modifiable group, such as a chloroketone moiety, an aldehyde moiety, or an epoxide moiety. Other examples of trypsin inhibitors are aprotinin, camostat and pentamidine.

Other examples of trypsin inhibitors include compounds of formula:

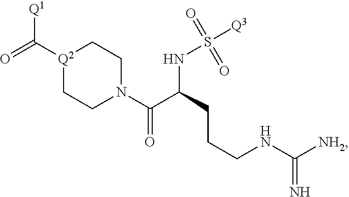

wherein:

Q¹ is selected from —O-Q⁴ or -Q⁴-COOH, where Q⁴ is C₁-C₄ alkyl;

Q² is N or CH; and

Q³ is aryl or substituted aryl.

Certain trypsin inhibitors include compounds of formula:

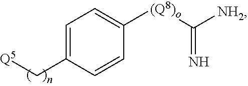

wherein:

Q⁵ is —C(O)—COOH or —NH-Q⁶-Q⁷-SO₂—C₆H₅, where

Q⁶ is —(CH₂)ₚ—COOH;

Q⁷ is —(CH₂)ᵣ—C₆H₅;

Q⁸ is NH;

n is a number from zero to two;

o is zero or one;

p is an integer from one to three; and

r is an integer from one to three.

Other examples of trypsin inhibitors include compounds of formula:

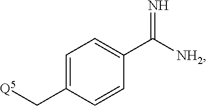

wherein:

Q⁵ is —C(O)—COOH or —NH-Q⁶-Q⁷-SO₂—C₆H₅, where

Q⁶ is —(CH₂)ₚ—COOH;

Q⁷ is —(CH₂)ᵣ—C₆H₅; and

p is an integer from one to three; and

r is an integer from one to three.

Certain trypsin inhibitors include the following:

| | | |
|---|---|---|
| Compound 101 | 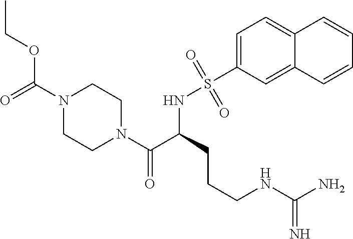 | (S)-ethyl 4-(5-guanidino-2- (naphthalene-2- sulfonamido)pentanoyl)piperazine- 1-carboxylate |
| Compound 102 | 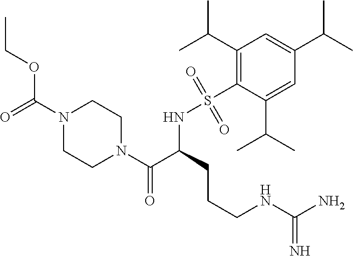 | (S)-ethyl 4-(5-guanidino-2-(2,4,6- triisopropylphenyl- sulfonamido)pentanoyl)piperazine- 1-carboxylate |
| Compound 103 | 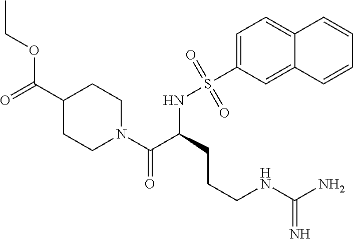 | (S)-ethyl 1-(5-guanidino-2- (naphthalene-2- sulfonamido)pentanoyl)piperidine- 4-carboxylate |
| Compound 104 | 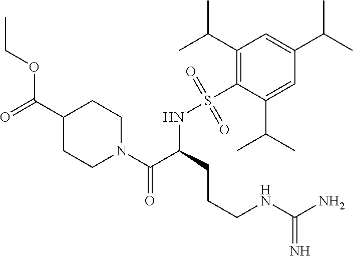 | (S)-ethyl 1-(5-guanidino-2-(2,4,6- triisopropylphenyl- sulfonamido)pentanoyl)piperidine- 4-carboxylate |
| Compound 105 | 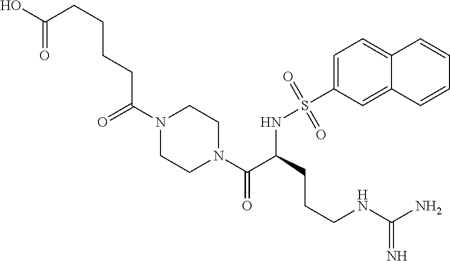 | (S)-6-(4-(5-guanidino-2- (naphthalene-2- sulfonamido)pentanoyl)piperazin- 1-yl)-6-oxohexanoic acid |
| Compound 106 | 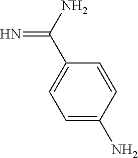 | 4-aminobenzimidamide (also 4-aminobenzamidine) |
| Compound 107 | 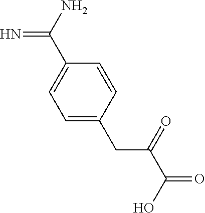 | 3-(4-carbamimidoylphenyl)-2- oxopropanoic acid |
| Compound 108 | 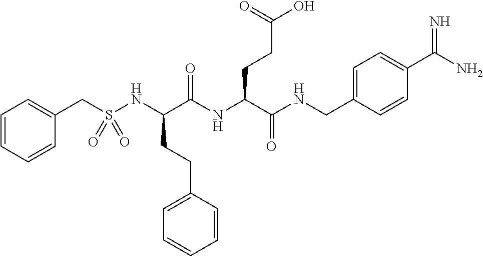 | (S)-5-(4- carbamimidoylbenzylamino)-5- oxo-4-((R)-4-phenyl-2- (phenylmethylsulfonamido)butana- mido)pentanoic acid |
| Compound 109 | 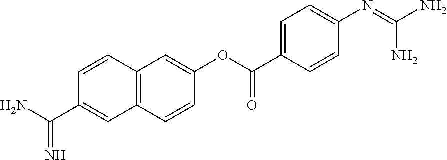 | 6-carbamimidoylnaphthalen-2-yl 4- (diaminomethyleneamino)benzoate |
| Compound 110 | 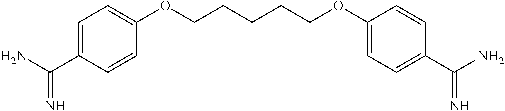 | 4,4′-(pentane-1,5- diylbis(oxy))dibenzimidamide |

A description of methods to prepare Compound 101, Compound 102, Compound 103, Compound 104, Compound 105, Compound 107, and Compound 108 is provided in PCT International Publication Number WO 2010/045599A1, published 22 Apr. 2010, which is hereby incorporated by reference in its entirety. Compound 106, Compound 109, and Compound 110 can be obtained commercially (Sigma-Aldrich, St. Louis, Mo., USA.).

In certain embodiments, the trypsin inhibitor is SBTI, BBSI, Compound 101, Compound 106, Compound 108, Compound 109, or Compound 110. In certain embodiments, the trypsin inhibitor is camostat.

In certain embodiments, the trypsin inhibitor is a compound of formula T-I:

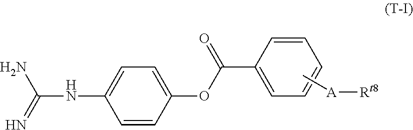

wherein

A represents a group of the following formula:

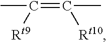

R^{t9} and R^{t10} each represents independently a hydrogen atom or a C₁₋₄ alkyl group,

R^{t8} represents a group selected from the following formulae:

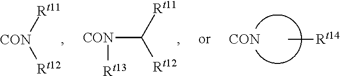

wherein R^{t11}, R^{t12} and R^{t13} each represents independently

(1) a hydrogen atom,

(2) a phenyl group,

(3) a C₁₋₄ alkyl group substituted by a phenyl group,

(4) a C₁₋₁₀ alkyl group,

(5) a C₁₋₁₀ alkoxyl group,

(6) a C₂₋₁₀ alkenyl group having 1 to 3 double bonds,

(7) a C₂₋₁₀ alkynyl group having 1 to 2 triple bonds,

(8) a group of formula: R^{t15}—C(O)XR^{t16},
wherein R^{t15} represents a single bond or a C₁₋₈ alkylene group,
X represents an oxygen atom or an NH-group, and
R^{t16} represents a hydrogen atom, a C₁₋₄ alkyl group, a phenyl group or a C₁₋₄ alkyl group substituted by a phenyl group, or

(9) a C₃₋₇ cycloalkyl group;

the structure

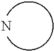

represents a 4-7 membered monocyclic hetero-ring containing 1 to 2 nitrogen or oxygen atoms,

R^{t14} represents a hydrogen atom, a C₁₋₄ alkyl group substituted by a phenyl group or a group of formula: COOR^{t17}, wherein R^{t17} represents a hydrogen atom, a C₁₋₄ alkyl group or a C₁₋₄ alkyl group substituted by a phenyl group;

provided that R^{t11}, R^{t12} and R^{t13} do not represent simultaneously hydrogen atoms;

or nontoxic salts, acid addition salts or hydrates thereof.

In certain embodiments, the trypsin inhibitor is a compound selected from the following:

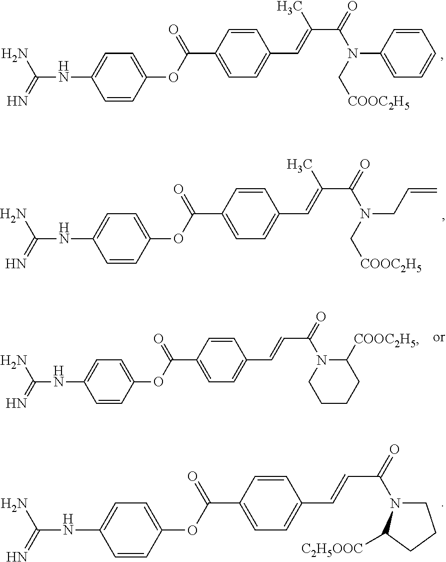

In certain embodiments, the trypsin inhibitor is a compound of formula T-II:

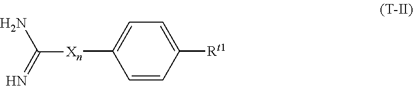

wherein

X is NH;

n is zero or one; and

R^{t1} is selected from hydrogen, halogen, nitro, alkyl, substituted alkyl, alkoxy, carboxyl, alkoxycarbonyl, acyl, aminoacyl, guanidine, amidino, carbamide, amino, substituted amino, hydroxyl, cyano and —(CH₂)ₘ—C(O)—O—(CH₂)ₘ—C(O)—N—Rⁿ¹Rⁿ², wherein each m is independently zero to 2; and Rⁿ¹ and Rⁿ² are independently selected from hydrogen and C₁₋₄ alkyl.

In certain embodiments, in formula T-II, R^{t1} is guanidino or amidino.

In certain embodiments, in formula T-II, R^{t1} is —(CH₂)ₘ—C(O)—O—(CH₂)ₘ—C(O)—N—Rⁿ¹Rⁿ², wherein m is one and Rⁿ¹ and Rⁿ² are methyl.

In certain embodiments, the trypsin inhibitor is a compound of formula T-III:

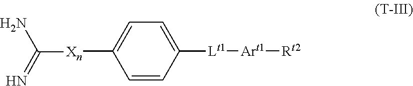

wherein

X is NH;

n is zero or one;

L^{t1} is selected from —C(O)—O—; —O—C(O)—; —O—(CH₂)ₘ—O—; —OCH₂—Ar^{t2}—CH₂O—; —C(O)—NR^{t3}—; and —NR^{t3}—C(O)—;

R^{t3} is selected from hydrogen, C₁₋₆ alkyl, and substituted C₁₋₆ alkyl;

Ar^{t1} and Ar^{t2} are independently a substituted or unsubstituted aryl group;

m is a number from 1 to 3; and

R^{t2} is selected from hydrogen, halogen, nitro, alkyl, substituted alkyl, alkoxy, carboxyl, alkoxycarbonyl, acyl, aminoacyl, guanidine, amidino, carbamide, amino, substituted amino, hydroxyl, cyano and —(CH₂)ₘ—C(O)—O—(CH₂)ₘ—C(O)—N—Rⁿ¹Rⁿ², wherein each m is independently zero to 2; and Rⁿ¹ and Rⁿ² are independently selected from hydrogen and C₁₋₄ alkyl.

In certain embodiments, in formula T-III, R^{t2} is guanidino or amidino.

In certain embodiments, in formula T-III, R^{t2} is —(CH₂)ₘ—C(O)—O—(CH₂)ₘ—C(O)—N—Rⁿ¹Rⁿ², wherein m is one and Rⁿ¹ and Rⁿ² are methyl.

In certain embodiments, the trypsin inhibitor is a compound of formula T-IV:

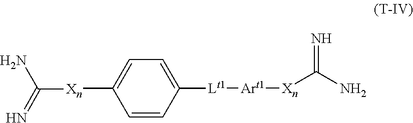

wherein

each X is NH;

each n is independently zero or one;

L^{t1} is selected from —C(O)—O—; —O—C(O)—; —O—(CH₂)ₘ—O—; —OCH₂—Ar^{t2}—CH₂O—; —C(O)—NR^{t3}—; and —NR^{t3}—C(O)—;

R^{t3} is selected from hydrogen, C₁₋₆ alkyl, and substituted C₁₋₆ alkyl;

Ar^{t1} and Ar^{t2} are independently a substituted or unsubstituted aryl group; and

m is a number from 1 to 3.

In certain embodiments, in formula T-IV, Ar^{t1} or Ar^{t2} is phenyl.

In certain embodiments, in formula T-IV, Ar^{t1} or Ar^{t2} is naphthyl.

In certain embodiments, the trypsin inhibitor is Compound 109.

In certain embodiments, the trypsin inhibitor is

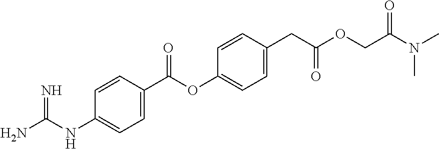

In certain embodiments, the trypsin inhibitor is Compound 110 or a bis-arylamidine variant thereof; see, for example, J. D. Geratz, M. C.-F. Cheng and R. R. Tidwell (1976) J. Med. Chem. 19, 634-639.

It will be appreciated that the pharmaceutical composition according to the embodiments may further comprise one or more additional trypsin inhibitors.

It is to be appreciated that the invention also includes inhibitors of other enzymes involved in protein assimilation that can be used in combination with a prodrug disclosed herein comprising an amino acid of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine or amino acid variants thereof.

### Combinations of GABA_{A} Agonists and GI Enzyme Inhibitors

Examples of compositions containing GABA_{A} agonist and a GI enzyme inhibitor (e.g., a trypsin inhibitor) are described below.

The embodiments provide a pharmaceutical composition, which comprises a GI enzyme inhibitor and a GABA_{A} agonist selected from benzodiazepines, non-benzodiazepines, barbiturates, neuroactive steroids, methaqualone, progabide, and tiagabine. In certain embodiments, the pharmaceutical composition comprises a GI enzyme inhibitor and a benzodiazepine. In certain embodiments, the pharmaceutical composition comprises a GI enzyme inhibitor and a non-benzodiazepine. In certain embodiments, the pharmaceutical composition comprises a GI enzyme inhibitor and a barbiturate. In certain embodiments, the pharmaceutical composition comprises a GI enzyme inhibitor and a neuroactive steroid. In certain embodiments, the pharmaceutical composition comprises a GI enzyme inhibitor and methaqualone. In certain embodiments, the pharmaceutical composition comprises a GI enzyme inhibitor and progabide. In certain embodiments, the pharmaceutical composition comprises a GI enzyme inhibitor and tiagabine.

The embodiments provide a pharmaceutical composition, which comprises a trypsin inhibitor and a GABA_{A} agonist selected from benzodiazepines, non-benzodiazepines, barbiturates, neuroactive steroids, methaqualone, progabide, and tiagabine. In certain embodiments, the pharmaceutical composition comprises a trypsin inhibitor and a benzodiazepine. In certain embodiments, the pharmaceutical composition comprises a trypsin inhibitor and a non-benzodiazepine. In certain embodiments, the pharmaceutical composition comprises a trypsin inhibitor and a barbiturate. In certain embodiments, the pharmaceutical composition comprises a trypsin inhibitor and a neuroactive steroid. In certain embodiments, the pharmaceutical composition comprises a trypsin inhibitor and methaqualone. In certain embodiments, the pharmaceutical composition comprises a trypsin inhibitor and progabide. In certain embodiments, the pharmaceutical composition comprises a trypsin inhibitor and tiagabine.

The embodiments provide a pharmaceutical composition, which comprises a compound of Formulae T-I to T-IV and a GABA_{A} agonist selected from benzodiazepines, non-benzodiazepines, barbiturates, neuroactive steroids, methaqualone, progabide, and tiagabine. In certain embodiments, the pharmaceutical composition comprises a compound of Formulae T-I to T-IV and a benzodiazepine. In certain embodiments, the pharmaceutical composition comprises a compound of Formulae T-I to T-IV and a non-benzodiazepine. In certain embodiments, the pharmaceutical composition comprises a compound of Formulae T-I to T-IV and a barbiturate. In certain embodiments, the pharmaceutical composition comprises a compound of Formulae T-I to T-IV and a neuroactive steroid. In certain embodiments, the pharmaceutical composition comprises a compound of Formulae T-I to T-IV and methaqualone. In certain embodiments, the pharmaceutical composition comprises a compound of Formulae T-I to T-IV and progabide. In certain embodiments, the pharmaceutical composition comprises a compound of Formulae T-I to T-IV and tiagabine.

The embodiments provide a pharmaceutical composition, which comprises Compound 109 and a GABA_{A} agonist selected from benzodiazepines, non-benzodiazepines, barbiturates, neuroactive steroids, methaqualone, progabide, and tiagabine. In certain embodiments, the pharmaceutical composition comprises Compound 109 and a benzodiazepine. In certain embodiments, the pharmaceutical composition comprises Compound 109 and a non-benzodiazepine. In certain embodiments, the pharmaceutical composition comprises Compound 109 and a barbiturate. In certain embodiments, the pharmaceutical composition comprises Compound 109 and a neuroactive steroid. In certain embodiments, the pharmaceutical composition comprises Compound 109 and methaqualone. In certain embodiments, the pharmaceutical composition comprises Compound 109 and progabide. In certain embodiments, the pharmaceutical composition comprises Compound 109 and tiagabine.

Certain embodiments provide for a combination of a GABA_{A} agonist and a trypsin inhibitor, shown in the table below.

| | | | Neuroactive |
|---|---|---|---|
| Benzodiazepine | Non-benzodiazepine | Barbiturate | steroid |
| and | and Trypsin | and Trypsin | and Trypsin |
| Trypsin Inhibitor | Inhibitor | Inhibitor | Inhibitor |
| SBTI | SBTI | SBTI | SBTI |
| BBSI | BBSI | BBSI | BBSI |
| Compound 101 | Compound 101 | Compound 101 | Compound 101 |
| Compound 106 | Compound 106 | Compound 106 | Compound 106 |
| Compound 108 | Compound 108 | Compound 108 | Compound 108 |
| Compound 109 | Compound 109 | Compound 109 | Compound 109 |
| Compound 110 | Compound 110 | Compound 110 | Compound 110 |

| Methaqualone and | Progabide and | Tiagabine and |
|---|---|---|
| Trypsin Inhibitor | Trypsin Inhibitor | Trypsin Inhibitor |
| SBTI | SBTI | SBTI |
| BBSI | BBSI | BBSI |
| Compound 101 | Compound 101 | Compound 101 |
| Compound 106 | Compound 106 | Compound 106 |
| Compound 108 | Compound 108 | Compound 108 |
| Compound 109 | Compound 109 | Compound 109 |
| Compound 110 | Compound 110 | Compound 110 |

The embodiments provide a pharmaceutical composition, which comprises (a) a GI enzyme inhibitor and (b) a CNS depressant, a muscle relaxant, an antihistamine, a drug for high blood pressure, an anti-psychotic, a pain medicine, an anti-seizure drug, a stimulant, or a veratrum alkaloid.

In certain embodiments, the pharmaceutical composition comprises a GI enzyme inhibitor and a CNS depressant. In certain embodiments, the pharmaceutical composition comprises a GI enzyme inhibitor and a muscle relaxant. In certain embodiments, the pharmaceutical composition comprises a GI enzyme inhibitor and an antihistamine. In certain embodiments, the pharmaceutical composition comprises a GI enzyme inhibitor and a drug for high blood pressure. In certain embodiments, the pharmaceutical composition comprises a GI enzyme inhibitor and an anti-psychotic. In certain embodiments, the pharmaceutical composition comprises a GI enzyme inhibitor and a pain medicine. In certain embodiments, the pharmaceutical composition comprises a GI enzyme inhibitor and an anti-seizure drug. In certain embodiments, the pharmaceutical composition comprises a GI enzyme inhibitor and a stimulant. In certain embodiments, the pharmaceutical composition comprises a GI enzyme inhibitor and a veratrum alkaloid.

The embodiments provide a pharmaceutical composition, which comprises (a) a trypsin inhibitor and (b) a CNS depressant, a muscle relaxant, an antihistamine, a drug for high blood pressure, an anti-psychotic, a pain medicine, an anti-seizure drug, a stimulant, or a veratrum alkaloid.

In certain embodiments, the pharmaceutical composition comprises a trypsin inhibitor and a CNS depressant. In certain embodiments, the pharmaceutical composition comprises a trypsin inhibitor and a muscle relaxant. In certain embodiments, the pharmaceutical composition comprises a trypsin inhibitor and an antihistamine. In certain embodiments, the pharmaceutical composition comprises a trypsin inhibitor and a drug for high blood pressure.

In certain embodiments, the pharmaceutical composition comprises a trypsin inhibitor and an anti-psychotic. In certain embodiments, the pharmaceutical composition comprises a trypsin inhibitor and a pain medicine. In certain embodiments, the pharmaceutical composition comprises a trypsin inhibitor and an anti-seizure drug. In certain embodiments, the pharmaceutical composition comprises a trypsin inhibitor and a stimulant. In certain embodiments, the pharmaceutical composition comprises a trypsin inhibitor and a veratrum alkaloid.

The embodiments provide a pharmaceutical composition, which comprises (a) a compound of Formulae T-I to T-IV and (b) a CNS depressant, a muscle relaxant, an antihistamine, a drug for high blood pressure, an anti-psychotic, a pain medicine, an anti-seizure drug, a stimulant, or a veratrum alkaloid.

In certain embodiments, the pharmaceutical composition comprises a compound of Formulae T-I to T-IV and a CNS depressant. The embodiments provide a pharmaceutical composition, which comprises a compound of Formulae T-I to T-IV and a CNS depressant selected from muscle relaxants, antihistamines, drugs for high blood pressure, anti-psychotics, pain medicines, anti-seizure drugs, stimulants, and veratrum alkaloids.

In certain embodiments, the pharmaceutical composition comprises a compound of Formulae T-I to T-IV and a muscle relaxant. In certain embodiments, the pharmaceutical composition comprises a compound of Formulae T-I to T-IV and an antihistamine. In certain embodiments, the pharmaceutical composition comprises a compound of Formulae T-I to T-IV and a drug for high blood pressure. In certain embodiments, the pharmaceutical composition comprises a compound of Formulae T-I to T-IV and an anti-psychotic. In certain embodiments, the pharmaceutical composition comprises a compound of Formulae T-Ito T-IV and a pain medicine. In certain embodiments, the pharmaceutical composition comprises a compound of Formulae T-I to T-IV and an anti-seizure drug. In certain embodiments, the pharmaceutical composition comprises a compound of Formulae T-I to T-IV and a stimulant. In certain embodiments, the pharmaceutical composition comprises a compound of Formulae T-I to T-IV and a veratrum alkaloid.

The embodiments provide a pharmaceutical composition, which comprises (a) Compound 109 and (b) a CNS depressant, a muscle relaxant, an antihistamine, a drug for high blood pressure, an anti-psychotic, a pain medicine, an anti-seizure drug, a stimulant, or a veratrum alkaloid.

In certain embodiments, the pharmaceutical composition comprises Compound 109 and a CNS depressant. In certain embodiments, the pharmaceutical composition comprises Compound 109 and a muscle relaxant. In certain embodiments, the pharmaceutical composition comprises Compound 109 and an antihistamine. In certain embodiments, the pharmaceutical composition comprises Compound 109 and a drug for high blood pressure. In certain embodiments, the pharmaceutical composition comprises Compound 109 and an anti-psychotic. In certain embodiments, the pharmaceutical composition comprises Compound 109 and a pain medicine. In certain embodiments, the pharmaceutical composition comprises Compound 109 and an anti-seizure drug. In certain embodiments, the pharmaceutical composition comprises Compound 109 and a stimulant. In certain embodiments, the pharmaceutical composition comprises Compound 109 and a veratrum alkaloid.

Certain embodiments provide for a combination of a CNS depressant and a trypsin inhibitor, shown in the table below.

| | Muscle | | Drugs for high | |
|---|---|---|---|---|
| CNS depressant | relaxants and | Antihistamines | blood pressure | Anti-psychotics |
| and Trypsin | Trypsin | and Trypsin | and Trypsin | and Trypsin |
| Inhibitor | Inhibitor | Inhibitor | Inhibitor | Inhibitor |
| SBTI | SBTI | SBTI | SBTI | SBTI |
| BBSI | BBSI | BBSI | BBSI | BBSI |
| Compound 101 | Compound 101 | Compound 101 | Compound 101 | Compound 101 |
| Compound 106 | Compound 106 | Compound 106 | Compound 106 | Compound 106 |
| Compound 108 | Compound 108 | Compound 108 | Compound 108 | Compound 108 |
| Compound 109 | Compound 109 | Compound 109 | Compound 109 | Compound 109 |
| Compound 110 | Compound 110 | Compound 110 | Compound 110 | Compound 110 |

| Pain medicines and | Anti-seizure drugs | Stimulants and | Veratrum alkaloids |
|---|---|---|---|
| Trypsin Inhibitor | and Trypsin Inhibitor | Trypsin Inhibitor | and Trypsin Inhibitor |
| SBTI | SBTI | SBTI | SBTI |
| BBSI | BBSI | BBSI | BBSI |
| Compound 101 | Compound 101 | Compound 101 | Compound 101 |
| Compound 106 | Compound 106 | Compound 106 | Compound 106 |
| Compound 108 | Compound 108 | Compound 108 | Compound 108 |
| Compound 109 | Compound 109 | Compound 109 | Compound 109 |
| Compound 110 | Compound 110 | Compound 110 | Compound 110 |

Prodrugs that Release Second Drugs

In certain embodiments, the first drug interacts in an additive or synergistic manner with a second drug to produce an adverse drug-drug interaction. The second drug is released from a GI enzyme-cleavable prodrug. A “GI enzyme-cleavable prodrug” is a prodrug that comprises a promoiety comprising a GI enzyme-cleavable moiety. A GI enzyme-cleavable moiety has a site that is susceptible to cleavage by a GI enzyme. In certain embodiments, the second drug is a drug that is susceptible to misuse, abuse, or overdose, such as an opioid, amphetamine, or an amphetamine analog. In one embodiment, prodrug that releases the second drug is not a peptide. In one embodiment, the prodrug that releases the second drug is not a protein. In one embodiment, the second drug is not a peptide. In one embodiment, the second drug is not a protein.

Examples of GI enzyme-cleavable opioid prodrugs and GI enzyme-cleavable amphetamine prodrugs are disclosed herein.

### Opioid Prodrugs

In certain embodiments, the GI enzyme-cleavable prodrug is a GI enzyme-cleavable opioid prodrug. Examples of opioid prodrugs are described below.

An “opioid” refers to a chemical substance that exerts its pharmacological action by interaction at an opioid receptor. An opioid can be a natural product, a synthetic compound or a semi-synthetic compound. In certain embodiments, an opioid is a compound with a pharmacophore that presents to the opioid receptor an aromatic group and an aliphatic amine group in an architecturally discrete way. See, for example, Foye's Principles of Medicinal Chemistry, Sixth Edition, ed. T. L. Lemke and D. A. Williams, Lippincott Williams & Wilkins, 2008, particularly Chapter 24, pages 653-678.

A phenolic opioid refers to a subset of the opioids that contain a phenol group. For instance, the following opioids contain a phenol group that can be a point of attachment to a promoiety: buprenorphine, dihydroetorphine, diprenorphine, etorphine, hydromorphone, levorphanol, morphine, nalmefene, naloxone, N-methyldiprenorphine, N-methylnaloxone, naltrexone, N-methylnaltexone, oxymorphone, oripavine, ketobemidone, dezocine, pentazocine, phenazocine, butorphanol, nalbuphine, meptazinol, o-desmethyltramadol, tapentadol, and nalorphine. The following opioids also contain a phenol that can be a point of attachment to a promoiety: benzylmorphine, codeine, dihydrocodeine, dihydromorphine, ethylmorphine, loperamide, methyldihydromorphine, normorphine, N-methylnalmefene, olmefentanyl, oxycodone, pentamorphone, pholcodine, and tramadol.

A ketone-containing opioid refers to a subset of the opioids that contain a ketone group. For instance, the following opioids contain a ketone group that can be a point of attachment to a promoiety: acetylmorphone, hydrocodone, hydromorphone, ketobemidone, methadone, naloxone, N-methylnaloxone, naltrexone, N-methylnaltrexone, oxycodone, oxymorphone, and pentamorphone.

An amino-containing opioid refers to a subset of the opioids that contain an amino group. For instance, the following opioids contain an amino group that can be a point of attachment to a promoiety as a quaternary ammonium salt: acetylmorphine, alfentanil, benzylmorphine, buprenorphine, butorphanol, carfentanil, codeine, dextropropoxyphene, diacetylhidhydromorphine, diacetylmorphine, dihydrocodeine, dihydrocodeinone enol acetate, dihydroetorphine, dihydromorphine, diphenoxylate, diprenorphine, dipropanoylmorphine, ethylmorphine, etorphine, fentanyl, hydrocodone, hydromorphone, ketobemidone, leva-α-acetylmethadol, levorphanol, lofentanil, meperidine, meptazinol, methadone, methyldihydromorphine, morphine, nalbuphine, nalmefene, nalorphine, naloxone, naltrexone, nicocodeine, nicomorpine, normorphine, olmefentanyl, oripavin, oxycodone, oxymorphone, pentamorphone, pentazocine, phenazocine, pholcodine, remifentanil, sufentanil, tapentadol, thebaine, tilidine, tramadol, and o-desmethyltramadol. For instance, the following opioid contains an amino group that can be a point of attachment to a promoiety: dezozine.

An amide-containing opioid refers to a subset of the opioids that contain an amide group. For instance, the following opioids contain an amide group that can be a point of attachment to a promoiety: alfentanil, carfentanil, fentanyl, lofentanil, loperamide, olmefentanyl, remifentanil, and sufentanil.

It is contemplated that opioids bearing at least some of the functionalities described herein will be developed; such opioids are included as part of the scope of this disclosure.

In certain embodiments, a promoiety can be attached to a phenolic opioid via modification of the phenol moiety. Release of the opioid is mediated by enzymatic cleavage of the promoiety from the phenolic opioid. In certain embodiments, a promoiety can be attached to a ketone-containing opioid through the enolic oxygen atom of the ketone moiety. Release of the opioid is mediated by enzymatic cleavage of the promoiety from the ketone-containing opioid. In certain embodiments, a promoiety can be attached to an amide-containing opioid through the enolic oxygen of the amide moiety or the imine tautomer. Release of the opioid is mediated by enzymatic cleavage of the promoiety from the amide-containing opioid. In each case, the promoiety comprises an enzyme-cleavable moiety that is susceptible to cleavage by a GI enzyme. Such cleavage can initiate, contribute to or effect drug release.

### Phenol-modified Opioid Prodrugs

The disclosure provides a phenol-modified opioid prodrug which provides enzymatically-controlled release of a phenolic opioid. In a phenol-modified opioid prodrug, a promoiety is attached to the phenolic opioid via modification of the phenol moiety. A phenol-modified opioid prodrug can also be referred to as a phenolic opioid prodrug. In a phenol-modified opioid prodrug, the hydrogen atom of the phenolic hydroxyl group of the phenolic opioid is replaced by a covalent bond to a promoiety.

As disclosed herein, a gastrointestinal (GI) enzyme-cleavable phenol-modified opioid prodrug is a phenol-modified opioid prodrug that comprises a promoiety comprising a GI enzyme-cleavable moiety having a site susceptible to cleavage by a GI enzyme. Such a prodrug comprises a phenolic opioid covalently bound to a promoiety comprising a GI enzyme-cleavable moiety, wherein cleavage of the GI enzyme-cleavable moiety by the GI enzyme mediates release of the drug. Cleavage can initiate, contribute to or effect drug release.

According to one aspect, the embodiments include compositions, which comprise compounds disclosed in WO 2007/140272, which is hereby incorporated by reference in its entirety. WO 2007/140272 describes the synthesis of phenol-modified opioid prodrugs with promoiety comprising cyclizable spacer leaving group and cleavable moiety.

In a particular embodiment, the present disclosure provides the compound hydromorphone 3-(N-methyl-N-(2-N′-acetylarginylamino)) ethylcarbamate, or a pharmaceutically acceptable salt thereof. This compound is described in Example 3 of WO 2007/140272.

Formulae PCC-(I)

According to one aspect, the embodiments include compositions, which comprise a compound of general formula PCC-(I):

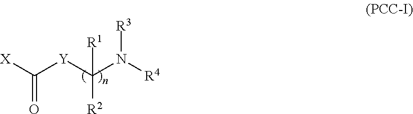

or a salt, hydrate or solvate thereof wherein:
X is a phenolic opioid, wherein the hydrogen atom of the phenolic hydroxyl group is replaced by a covalent bond to —C(O)—Y—(C(R¹)(R²))ₙ—N—(R³)(R⁴);
Y is NR⁵ and R⁵ is (1-4C)alkyl;
n is 2 or 3;
R¹ and R² are each hydrogen;
R³ is hydrogen or (1-4C)alkyl;
R⁴ is:
a residue of an L-amino acid selected from alanine, arginine, asparagine, aspartic acid, cysteine, glycine, glutamine, glutamic acid, histidine, isoleucine, leucine, methionine, phenylalanine, proline, serine, threonine, tryptophan, tyrosine, lysine and valine;
a residue of a dipeptide or tripeptide composed of two or three L-amino acid residues selected independently from alanine, arginine, asparagine, aspartic acid, cysteine, glycine, glutamine, glutamic acid, histidine, isoleucine, leucine, methionine, phenylalanine, proline, serine, threonine, tryptophan, tyrosine, lysine and valine; or
a residue of an N-acyl derivative thereof.

According to one aspect, the embodiments include compositions, which comprise compounds disclosed in US 2009/0137618, which is hereby incorporated by reference in its entirety. According to one aspect, the embodiments include compositions, which comprise compounds disclosed in WO 2010/045599, which is hereby incorporated by reference in its entirety.

Formula PC-(II)

The embodiments provide a composition, which comprises a compound of general formula PC-(II):

X—C(O)—NR¹—(C(R²)(R³))ₙ—NH—C(O)—CH(R⁴)—NH(R⁵) (PC-(II))

or a pharmaceutically acceptable salt thereof, in which:

X represents a residue of a phenolic opioid, wherein the hydrogen atom of the phenolic hydroxyl group is replaced by a covalent bond to —C(O)—NR¹—(C(R²)(R³))ₙ—NH—C(O)—CH(R⁴)—NH(R⁵);

R¹ is selected from alkyl, substituted alkyl, arylalkyl, substituted arylalkyl, aryl and substituted aryl;

each R² is independently selected from hydrogen, alkyl, substituted alkyl, aryl, substituted aryl, acyl, and aminoacyl;

each R³ is independently selected from hydrogen, alkyl, substituted alkyl, aryl, substituted aryl, acyl, and aminoacyl;

or R² and R³ together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;

n represents an integer from 2 to 4;

R⁴ represents —CH₂CH₂CH₂NH(C═NH)NH₂ or —CH₂CH₂CH₂CH₂NH₂, the configuration of the carbon atom to which R⁴ is attached corresponding with that in an L-amino acid; and

R⁵ represents a hydrogen atom, an N-acyl group (including N-substituted acyl), a residue of an amino acid, a dipeptide, or an N-acyl derivative (including N-substituted acyl derivative) of an amino acid or dipeptide.

Formula PC-(VIII)

The embodiments provide a composition, which comprises a compound of general formula PC-(VIII):

X—C(O)—NR¹—(C(R²)(R³))ₙ—NH—R⁶ (PC-(VIII))

or a pharmaceutically acceptable salt thereof, in which:

X represents a residue of a phenolic opioid, wherein the hydrogen atom of the phenolic hydroxyl group is replaced by a covalent bond to —C(O)—NR¹—(C(R²)(R³))ₙ—NHR⁶;

R¹ is selected from alkyl, substituted alkyl, arylalkyl, substituted arylalkyl, aryl and substituted aryl;

each R² is independently selected from hydrogen, alkyl, substituted alkyl, aryl, substituted aryl, acyl, and aminoacyl;

each R³ is independently selected from hydrogen, alkyl, substituted alkyl, aryl, substituted aryl, acyl, and aminoacyl;

or R² and R³ together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;

n represents an integer from 2 to 4; and

R⁶ is a trypsin-cleavable moiety.

Compound PC-5

The embodiments provide a composition, which comprises Compound PC-5, [2-((S)-2-malonylamino-6-amino-hexanoyl amino)-ethyl]-ethyl-carbamic acid hydromorphone ester, shown below:

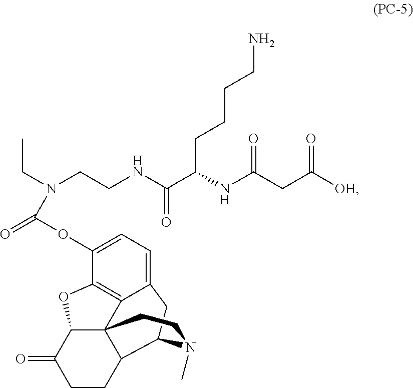

or acceptable salts, solvates, and hydrates thereof.

Formula TC-(I)

The embodiments provide a composition, which comprises a compound of general formula TC-(I):

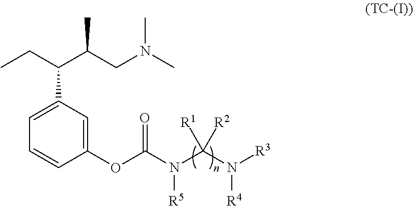

wherein:

R⁵ is selected from alkyl, substituted alkyl, arylalkyl, substituted arylalkyl, aryl and substituted aryl;

each R¹ is independently selected from hydrogen, alkyl, substituted alkyl, aryl, substituted aryl, acyl, and aminoacyl;

each R² is independently selected from hydrogen, alkyl, substituted alkyl, aryl, substituted aryl, acyl, and aminoacyl;

or R¹ and R² together with the carbon to which they are attached form a cycloalkyl, substituted cycloalkyl, aryl, or substituted aryl group, or two R¹ or R² groups on adjacent carbon atoms, together with the carbon atoms to which they are attached, form a cycloalkyl, substituted cycloalkyl, aryl, or substituted aryl group;

n is an integer from 2 to 4;

R³ is hydrogen;

R⁴ is

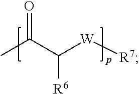

each R⁶ is independently selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or optionally, R⁶ and R⁷ together with the atoms to which they are bonded form a cycloheteroalkyl or substituted cycloheteroalkyl ring;

each W is independently —NR⁸—, —O— or —S—;

each R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;

p is an integer from one to 100; and

R⁷ is selected from hydrogen, alkyl, substituted alkyl, acyl, substituted acyl, alkoxycarbonyl, substituted alkoxycarbonyl, aryl, substituted aryl, arylalkyl, and substituted arylalkyl;

or a salt, hydrate or solvate thereof.

The disclosure provides for a compound of the following formula:

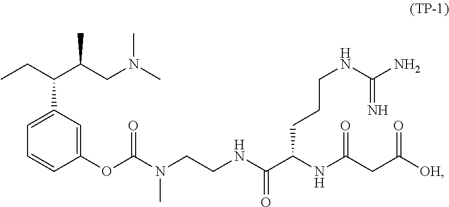

or a salt, hydrate or solvate thereof.

### Ketone-Modified Opioid Prodrugs

The disclosure provides a ketone-modified opioid prodrug which provides enzymatically-controlled release of a ketone-containing opioid. As used herein, a ketone-containing opioid is an opioid containing an enolizable ketone group. In a ketone-modified opioid prodrug, a promoiety is attached to the ketone-containing opioid through the enolic oxygen atom of the ketone moiety. In a ketone-modified opioid prodrug, the hydrogen atom of the corresponding enolic group of the ketone-containing opioid is replaced by a covalent bond to a promoiety.

As disclosed herein, a trypsin-cleavable ketone-modified opioid prodrug is a ketone-modified opioid prodrug that comprises a promoiety comprising a trypsin-cleavable moiety, i.e., a moiety having a site susceptible to cleavage by trypsin. Such a prodrug comprises a ketone-containing opioid covalently bound to a promoiety comprising a trypsin-cleavable moiety, wherein cleavage of the trypsin-cleavable moiety by trypsin mediates release of the drug. Cleavage can initiate, contribute to or effect drug release.

Formula KC-(III)

The embodiments provide a composition, which comprises a compound of general formula KC-(III):

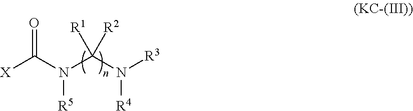

wherein:

X represents a residue of a ketone-containing opioid, wherein the hydrogen atom of the corresponding enolic group of the ketone is replaced by a covalent bond to —C(O)—NR⁵—(C(R¹)(R²))ₙ—NR³R⁴;

R⁵ is selected from alkyl, substituted alkyl, arylalkyl, substituted arylalkyl, aryl and substituted aryl;

each R¹ is independently selected from hydrogen, alkyl, substituted alkyl, aryl, substituted aryl, acyl, and aminoacyl;

each R² is independently selected from hydrogen, alkyl, substituted alkyl, aryl, substituted aryl, acyl, and aminoacyl;

or R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;

n is an integer from 2 to 4;

R³ is hydrogen;

R⁴ is

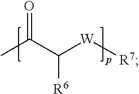

each R⁶ is independently selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or optionally, R⁶ and R⁷ together with the atoms to which they are bonded form a cycloheteroalkyl or substituted cycloheteroalkyl ring;

each W is independently —NR⁸—, —O— or —S—;

each R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;

p is an integer from one to 100; and

R⁷ is selected from hydrogen, alkyl, substituted alkyl, acyl, substituted acyl, alkoxycarbonyl, substituted alkoxycarbonyl, aryl, substituted aryl, arylalkyl, and substituted arylalkyl;

or a salt, hydrate or solvate thereof.

Compounds of formula KC-(IV) are compounds of formula KC-(III) in which R⁵ is selected from (1-6C) alkyl, (1-6C) substituted alkyl, —(CH₂)_{q}(C₆H₄)—COOH, —(CH₂)_{q}(C₆H₄)—COOCH₃, and —(CH₂)_{q}(C₆H₄)—COOCH₂CH₃, where q is an integer from one to 10; n is 2 or 3; R³ is hydrogen; R⁴ is an L-amino acid or peptide, where the peptide can be comprised of L-amino acids. In one of its composition aspects, the present embodiments provide a compound of formula KC-(IV):

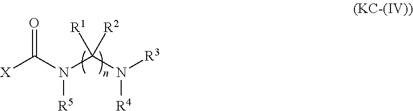

wherein:

X represents a residue of a ketone-containing opioid, wherein the hydrogen atom of the corresponding enolic group of the ketone is replaced by a covalent bond to —C(O)—NR⁵—(C(R¹)(R²))ₙ—NR³R⁴;

R⁵ is selected from (1-6C)alkyl, (1-6C) substituted alkyl, —(CH₂)_{q}(C₆H₄)—COOH, —(CH₂)_{q}(C₆H₄)—COOCH₃, and —(CH₂)_{q}(C₆H₄)—COOCH₂CH₃, where q is an integer from one to 10;

each R¹ is independently selected from hydrogen, alkyl, substituted alkyl, aryl, substituted aryl, acyl, and aminoacyl;

each R² is independently selected from hydrogen, alkyl, substituted alkyl, aryl, substituted aryl, acyl, and aminoacyl;

or R¹ and R² together with the carbon to which they are attached form a cycloalkyl or substituted cycloalkyl group, or two R¹ or R² groups on adjacent carbon atoms, together with the carbon atoms to which they are attached, form a cycloalkyl or substituted cycloalkyl group;

n is 2 or 3;

R³ is hydrogen;

R⁴ is a residue of an L-amino acid selected from alanine, arginine, asparagine, aspartic acid, cysteine, glycine, glutamine, glutamic acid, histidine, isoleucine, leucine, lysine, methionine, phenylalanine, proline, serine, threonine, tryptophan, tyrosine and valine, or a residue of an N-acyl derivative of any of said amino acids; or a residue of a peptide composed of at least two L-amino acid residues selected independently from alanine, arginine, asparagine, aspartic acid, cysteine, glycine, glutamine, glutamic acid, histidine, isoleucine, leucine, lysine, methionine, phenylalanine, proline, serine, threonine, tryptophan, tyrosine and valine or a residue of an N-acyl derivative thereof;

or a salt, hydrate or solvate thereof.

Compounds of formula KC-(Va) are compounds of formula KC-(III) in which R⁴ is a trypsin-cleavable moiety. In one of its composition aspects, the present embodiments provide a compound of formula KC-(Va):

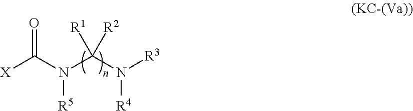

wherein:

X represents a residue of a ketone-containing opioid, wherein the hydrogen atom of the corresponding enolic group of the ketone is replaced by a covalent bond to —C(O)—NR⁵—(C(R¹)(R²))ₙ—NR³R⁴;

R⁵ is selected from alkyl, substituted alkyl, arylalkyl, substituted arylalkyl, aryl and substituted aryl;

each R¹ is independently selected from hydrogen, alkyl, substituted alkyl, aryl, substituted aryl, acyl, and aminoacyl;

each R² is independently selected from hydrogen, alkyl, substituted alkyl, aryl, substituted aryl, acyl, and aminoacyl;

or R¹ and R² together with the carbon to which they are attached form a cycloalkyl, substituted cycloalkyl, aryl, or substituted aryl group, or two R¹ or R² groups on adjacent carbon atoms, together with the carbon atoms to which they are attached, form a cycloalkyl, substituted cycloalkyl, aryl, or substituted aryl group;

n is an integer from 2 to 4;

R³ is hydrogen;

R⁴ is a trypsin-cleavable moiety;

or a salt, hydrate or solvate thereof.

Compounds of formula KC-(Vb) are compounds of formula KC-(III) in which R⁴ is a GI enzyme-cleavable moiety. In one of its composition aspects, the present embodiments provide a compound of formula KC-(Vb):

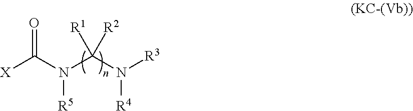

wherein:

X represents a residue of a ketone-containing opioid, wherein the hydrogen atom of the corresponding enolic group of the ketone is replaced by a covalent bond to —C(O)—NR⁵—(C(R¹)(R²))ₙ—NR³R⁴;

R⁵ is selected from alkyl, substituted alkyl, arylalkyl, substituted arylalkyl, aryl and substituted aryl;

each R¹ is independently selected from hydrogen, alkyl, substituted alkyl, aryl, substituted aryl, acyl, and aminoacyl;

each R² is independently selected from hydrogen, alkyl, substituted alkyl, aryl, substituted aryl, acyl, and aminoacyl;

or R¹ and R² together with the carbon to which they are attached form a cycloalkyl, substituted cycloalkyl, aryl, or substituted aryl group, or two R¹ or R² groups on adjacent carbon atoms, together with the carbon atoms to which they are attached, form a cycloalkyl, substituted cycloalkyl, aryl, or substituted aryl group;

n is an integer from 2 to 4;

R³ is hydrogen;

R⁴ is a GI enzyme-cleavable moiety;

or a salt, hydrate or solvate thereof.

Particular compounds of interest, and salts or solvates or stereoisomers thereof, include:

oxycodone 6-(N-methyl-N-(2-N′-acetylarginylamino))ethylcarbamate:

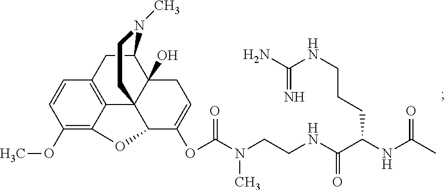

hydrocodone 6-(N-methyl-N-(2-N′-acetylarginylamino))ethylcarbamate:

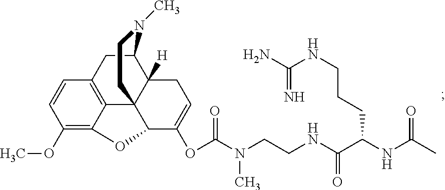

oxycodone 6-(N-methyl-N-(2-N′-malonylarginylamino))ethylcarbamate:

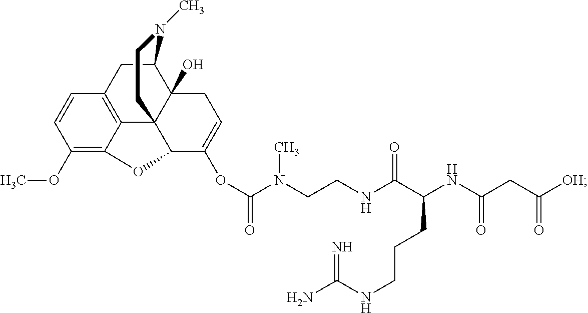

oxycodone 6-(N-5′-carboxypentyl-N-(2-N′-acetylarginylamino))ethylcarbamate:

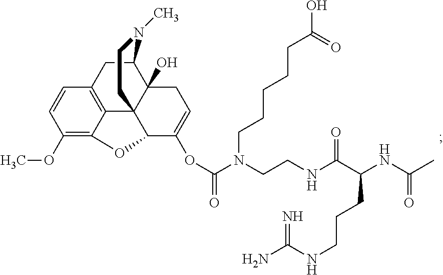

hydrocodone 6-(N-methyl-N-(2-N′-malonylarginylamino))ethylcarbamate:

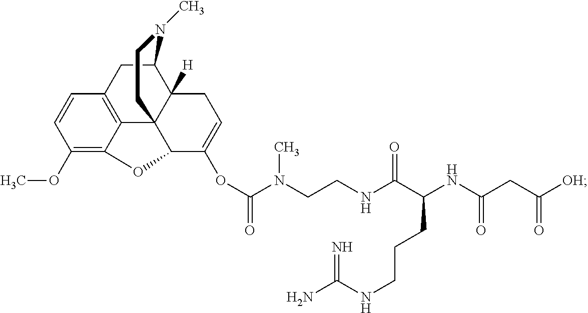

oxycodone 6-(N-methyl-N-(2-N′-acetylarginylamino-2-(N-methyl-N-carboxymethyl-acetamido))ethylcarbamate:

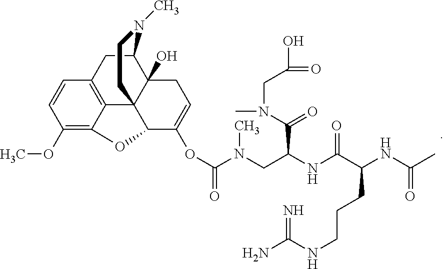

wherein the amino acid residue is of the L configuration.

Compound KC-8

The embodiments provide Compound KC-8, N-1-[3-(oxycodone-6-enol-carbonyl-methyl-amino)-2,2-dimethyl-propylamine]-arginine-glycine-malonate, shown below:

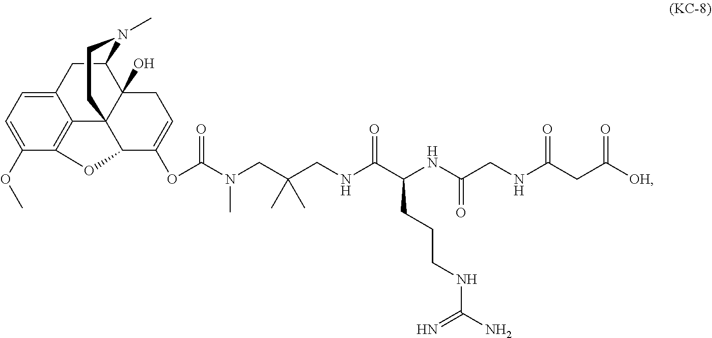

or acceptable salts, solvates, and hydrates thereof.

Compound KC-7

The embodiments provide Compound KC-7, N-1-[(S)-2-(oxycodone-6-enol-carbonyl-methyl-amino)-2-carbonyl-sarcosine-ethyl amine]-arginine-glycine-acetate, shown below:

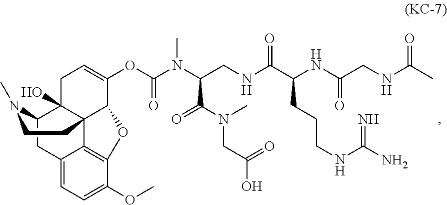

or acceptable salts, solvates, and hydrates thereof.

General Synthetic Procedures for Ketone-Modified Opioid Prodrugs

A representative synthesis for ketone-modified opioid prodrugs is shown in the following schemes. A representative synthesis for Compound KC203 is shown in Scheme KC-1. In Scheme KC-1, the terms R¹, R², R⁵, and n are defined herein. The terms PG¹ and PG² are amino protecting groups.

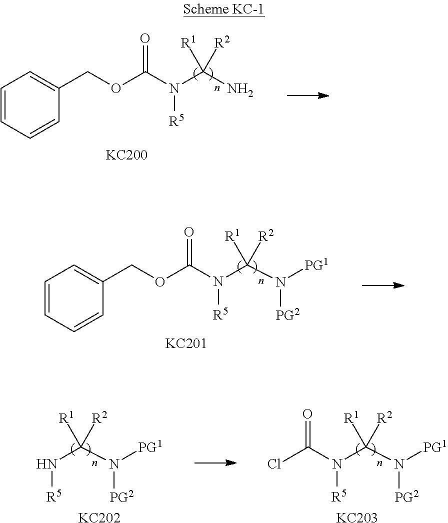

In Scheme KC-1, Compound KC200 is a commercially available starting material. Alternatively, Compound KC200 can be synthesized via a variety of different synthetic routes using commercially available starting materials and/or starting materials prepared by conventional synthetic methods.

With continued reference to Scheme KC-1, Compound KC200 is protected at the amino group to form Compound KC201, wherein PG¹ and PG² are amino protecting groups. Amino protecting groups can be found in T. W. Greene and P. G. M. Wuts, “Protective Groups in Organic Synthesis”, Fourth edition, Wiley, New York 2006. Representative amino-protecting groups include, but are not limited to, formyl groups; acyl groups, for example alkanoyl groups, such as acetyl; alkoxycarbonyl groups, such as tert-butoxycarbonyl (Boc); arylmethoxycarbonyl groups, such as benzyloxycarbonyl (Cbz) and 9-fluorenylmethoxycarbonyl (Fmoc); arylmethyl groups, such as benzyl (Bn), trityl (Tr), and 1,1-di-(4′-methoxyphenyl)methyl; silyl groups, such as trimethylsilyl (TMS) and tert-butyldimethylsilyl (TBS); and the like.

In certain embodiments, PG¹ and PG² are Boc groups. Conditions for forming Boc groups on Compound KC201 can be found in Greene and Wuts. One method is reaction of Compound KC200 with di-tert-butyl dicarbonate. The reaction can optionally be run in the presence of an activating agent, such as DMAP.

With continued reference to Scheme KC-1, the carboxybenzyl group on Compound KC201 is deprotected to form Compound KC202. Conditions to remove the carboxybenzyl group can be found in Greene and Wuts. Methods to remove the carboxybenzyl group include hydrogenolysis of Compound KC201 or treatment of Compound KC201 with HBr. One method to remove the carboxybenzyl group is reaction of Compound KC201 with hydrogen and palladium.

With continued reference to Scheme KC-1, Compound KC202 is reacted with phosgene to form Compound KC203. Reaction with phosgene forms an acyl chloride on the amino group of Compound KC202. Other reagents can act as substitutes for phosgene, such as diphosgene or triphosgene.

A representative synthesis for Compound KC302 is shown in Scheme KC-2. In Scheme 2, the terms R^{a}, R¹, R², R⁵, and n are defined herein. The terms PG¹ and PG² are amino protecting groups.

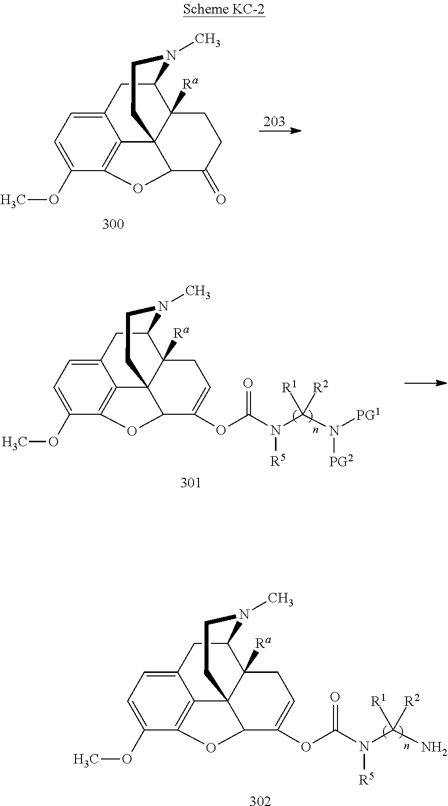

In Scheme KC-2, Compound KC300 is a commercially available starting material. Alternatively, Compound KC300 can be synthesized via a variety of different synthetic routes using commercially available starting materials and/or starting materials prepared by conventional synthetic methods.

With continued reference to Scheme KC-2, Compound KC300 is reacted with Compound KC203 to form Compound KC301. In this reaction, the enolate of Compound KC300 reacts with the acyl chloride of Compound KC203 to form a carbamate.

With continued reference to Scheme KC-2, the protecting groups PG¹ and PG² are removed from Compound KC301 to form Compound KC302. Conditions to remove amino groups can be found in Greene and Wuts. When PG¹ and PG² are Boc groups, the protecting groups can be removed with acidic conditions, such as treatment with trifluoroacetic acid.

A representative synthesis for Compound KC402 is shown in Scheme KC-3. In Scheme KC-3, the terms R^{a}, R¹, R², R⁵, R⁶, R⁷ and n are defined herein. The term PG³ is an amino protecting group.

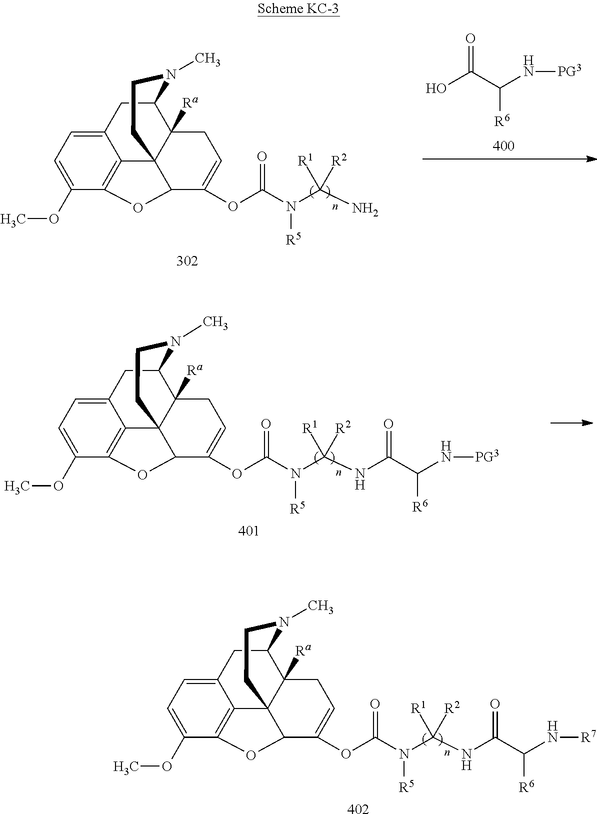

In Scheme KC-3, Compound KC400 is a commercially available starting material. Alternatively, Compound KC400 can be synthesized via a variety of different synthetic routes using commercially available starting materials and/or starting materials prepared by conventional synthetic methods.

With continued reference to Scheme KC-3, Compound KC302 reacts with Compound KC400 to form Compound KC401 in a peptide coupling reaction. A peptide coupling reaction typically employs a conventional peptide coupling reagent and is conducted under conventional coupling reaction conditions, typically in the presence of a trialkylamine, such as ethyldiisopropylamine or diisopropylethylamine (DIEA). Suitable coupling reagents for use include, by way of example, carbodiimides, such as ethyl-3-(3-dimethylamino)propylcarbodiimide (EDC), dicyclohexylcarbodiimide (DCC), diisopropylcarbodiimide (DIC) and the like, and other well-known coupling reagents, such as N,N′-carbonyldiimidazole, 2-ethoxy-1-ethoxycarbonyl-1,2-dihydroquinoline (EEDQ), benzotriazol-1-yloxy-tris(dimethylamino)phosphonium hexafluorophosphate (BOP), O-(7-azabenzotriazol-1-yl)-N,N,N,N′,N′-tetramethyluronium hexafluorophosphate (HATU) and the like. Optionally, well-known coupling promoters, such N-hydroxysuccinimide, 1-hydroxybenzotriazole (HOBT), 1-hydroxy-7-azabenzotriazole (HOAT), N,N-dimethylaminopyridine (DMAP) and the like, can be employed in this reaction. Typically, this coupling reaction is conducted at a temperature ranging from about 0° C. to about 60° C. for about 1 to about 72 hours in an inert diluent, such as THF or DMF. In certain instances, Compound KC302 reacts with Compound KC400 to form Compound KC401 in the presence of HATU and DIEA in DMF.

With continued reference to Scheme KC-3, Compound KC401 is transformed into Compound KC402 with removal of the amino protecting group and addition of R⁷ group. In certain cases, the amino protecting group is R⁷ and removal of the amino protecting group is optional.

As disclosed herein, representative amino-protecting groups include, but are not limited to, formyl groups; acyl groups, for example alkanoyl groups, such as acetyl; alkoxycarbonyl groups, such as tert-butoxycarbonyl (Boc); arylmethoxycarbonyl groups, such as benzyloxycarbonyl (Cbz) and 9-fluorenylmethoxycarbonyl (Fmoc); arylmethyl groups, such as benzyl (Bn), trityl (Tr), and 1,1-di-(4′-methoxyphenyl)methyl; silyl groups, such as trimethylsilyl (TMS) and tert-butyldimethylsilyl (TBS); and the like. In certain embodiments, PG³ is a Boc group. When PG³ is a Boc group, the protecting group can be removed with acidic conditions, such as treatment with trifluoroacetic acid.

In certain instances, the R⁷ group is added to Compound KC401. Conditions for addition of R⁷ depend on the identity of R⁷ and are known to those skilled in the art. In certain instances R⁷ is an acyl group, such as acetyl, benzoyl, malonyl, piperonyl or succinyl.

N-Acyl derivatives of the compounds of formula KC-(I) may conveniently be prepared by acylating a corresponding compound of formula KC-(I) using an appropriate acylating agent, for example an anhydride, such as acetic anhydride (to prepare an N-acetyl compound) or an acid halide. The reaction is conveniently performed in the presence of a non-reactive base, for example a tertiary amine, such as triethylamine. Convenient solvents include amides, such as dimethyl formamide. The temperature at which the reaction is performed is conveniently in the range of from 0 to 100° C., such as at ambient temperature.

With continued reference to Scheme KC-3, removal of other protecting groups can be performed if other protecting groups were used, such as protecting groups present on the R⁶ moiety. Conditions for removal of other protecting groups depend on the identity of the protecting group and are known to those skilled in the art. The conditions can also be found in Greene and Wuts.

Opioid Prodrugs with Heterocyclic Linkers

The embodiments provide a prodrug with a substituent which is a spacer leaving group bearing a nucleophilic nitrogen that is protected with an enzyme-cleavable moiety. Upon enzymatic cleavage of the cleavable moiety, the nucleophilic nitrogen is capable of forming a cyclic urea. A representative scheme of a cyclization of a spacer group is shown below, wherein X is an opioid.

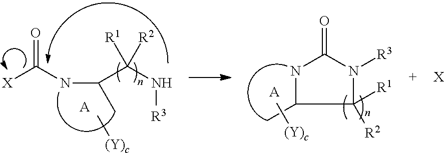

The rate of cyclization of the cyclic urea can be adjusted by incorporation of a heterocyclic ring within the spacer group. In certain embodiments, incorporation of a heterocyclic ring within the spacer group results in formation of a fused ring cyclic urea and in a faster cyclization reaction.

Formula HP-(I)

Compounds of the present disclosure include compounds of formula HP-(I) shown below. Compositions of the present disclosure also include compounds of formula HP-(I) shown below. Pharmaceutical compositions and methods of the present disclosure also contemplate compounds of formula HP-(I).

The present embodiments provide a compound of formula HP-(I):

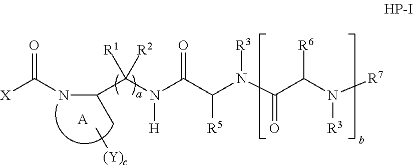

wherein

X is selected from a residue of a ketone-containing opioid, wherein the hydrogen atom of the corresponding hydroxyl group of the enolic tautomer of the ketone is replaced by a covalent bond to —C(O)—N[(A ring)-Y_{c}]-(CR¹R²)ₐ—NH—C(O)—CH(R⁵)—N(R³)—[C(O)—CH(R⁶)—N(R³)]_{b}—R⁷; a residue of a phenolic opioid, wherein the hydrogen atom of the phenolic hydroxyl group is replaced by a covalent bond to —C(O)—N[(A ring)-Y_{c}]-(CR¹R²)ₐ—NH—C(O)—CH(R⁵)—N(R³)—[C(O)—CH(R⁶)—N(R³)]_{b}—R⁷; and a residue of an amide-containing opioid, wherein —C(O)—N[(A ring)-Y_{c}]-(CR¹R²)ₐ—NH—C(O)—CH(R⁵)—N(R³)—[C(O)—CH(R⁶)—N(R³)]_{b}—R⁷ is connected to the amide-containing opioid through the oxygen of the amide group, wherein the amide group is converted to an amide enol or an imine tautomer;

the A ring is a heterocyclic 5 to 12-membered ring;

each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;

c is a number from zero to 3;

each R′ is independently selected from hydrogen, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;

each R² is independently selected from hydrogen, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano; or

R¹ and R² together with the carbon to which they are attached can form a cycloalkyl or substituted cycloalkyl group, or two R′ or R² groups on adjacent carbon atoms, together with the carbon atoms to which they are attached, can form a cycloalkyl or substituted cycloalkyl group;

a is an integer from one to 8;

provided that when a is one, the A ring is a heterocyclic 6 to 12-membered ring; and when the A ring is a heterocyclic 5-membered ring, then a is an integer from 2 to 8;

each R³ is independently hydrogen, alkyl, substituted alkyl, aryl or substituted aryl;

R⁵ is selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl;

each R⁶ is independently selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl;

b is a number from zero to 100; and

R⁷ is selected from hydrogen, alkyl, substituted alkyl, acyl, substituted acyl, alkoxycarbonyl, substituted alkoxycarbonyl, aryl, substituted aryl, arylalkyl, and substituted arylalkyl;

or a salt, hydrate or solvate thereof.

Ketone-Modified Opioid Prodrugs with Heterocyclic Linkers

The disclosure provides a ketone-modified opioid prodrug that provides controlled release of a ketone-containing opioid. In a ketone-modified opioid prodrug, a promoiety is attached to the ketone-containing opioid through the enolic oxygen atom of the ketone moiety. In a ketone-modified opioid prodrug, the hydrogen atom of the corresponding hydroxyl group of the enolic tautomer of the ketone-containing opioid is replaced by a covalent bond to a promoiety.

As disclosed herein, an enzyme-cleavable ketone-modified opioid prodrug is a ketone-modified opioid prodrug that comprises a promoiety comprising an enzyme-cleavable moiety, i.e., a moiety having a site susceptible to cleavage by an enzyme. In one embodiment, the cleavable moiety is a GI enzyme-cleavable moiety, such as a trypsin-cleavable moiety. Such a prodrug comprises a ketone-containing opioid covalently bound to a promoiety comprising an enzyme-cleavable moiety, wherein cleavage of the enzyme-cleavable moiety by an enzyme mediates release of the drug.

Formula HP-(II)

The present embodiments provide a compound of formula HP-(II):

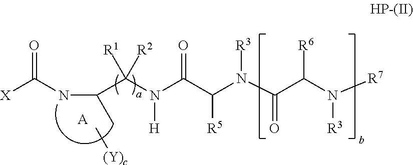

wherein

X represents a residue of a ketone-containing opioid, wherein the hydrogen atom of the corresponding hydroxyl group of the enolic tautomer of the ketone is replaced by a covalent bond to —C(O)—N[(A ring)-Y_{c}]—(CR¹R²)ₐ—NH—C(O)—CH(R⁵)—N(R³)—[C(O)—CH(R⁶)—N(R³)]_{b}—R⁷;

the A ring is a heterocyclic 5 to 12-membered ring;

each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;

c is a number from zero to 3;

each R¹ is independently selected from hydrogen, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;

each R² is independently selected from hydrogen, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano; or

R¹ and R² together with the carbon to which they are attached can form a cycloalkyl or substituted cycloalkyl group, or two R¹ or R² groups on adjacent carbon atoms, together with the carbon atoms to which they are attached, can form a cycloalkyl or substituted cycloalkyl group;

a is an integer from one to 8;

provided that when a is one, the A ring is a heterocyclic 6 to 12-membered ring; and when the A ring is a heterocyclic 5-membered ring, then a is an integer from 2 to 8;

each R³ is independently hydrogen, alkyl, substituted alkyl, aryl or substituted aryl;

R⁵ is selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl;

each R⁶ is independently selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl;

b is a number from zero to 100; and

R⁷ is selected from hydrogen, alkyl, substituted alkyl, acyl, substituted acyl, alkoxycarbonyl, substituted alkoxycarbonyl, aryl, substituted aryl, arylalkyl, and substituted arylalkyl;

or a salt, hydrate or solvate thereof.

Particular compounds of interest, and salts or solvates or stereoisomers thereof, include:

N-(oxycodone-6-enol-carbonyl)-R-(piperidine-2-methylamine)-L-arginine-glycine-malonate (Compound KC-17):

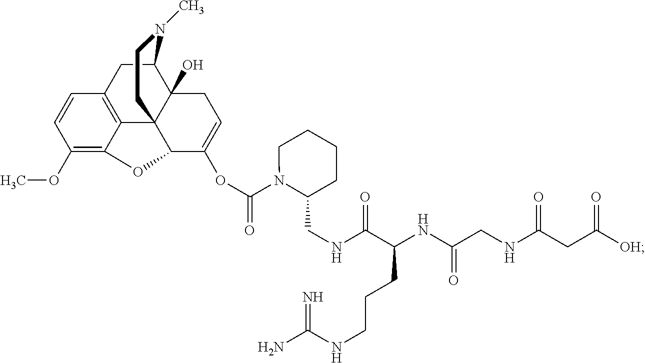

N-(oxycodone-6-enol-carbonyl)piperidine-2-methylamine-L-arginine-malonate (Compound KC-12):

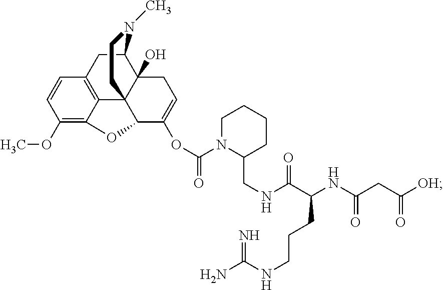

N-(oxycodone-6-enol-carbonyl)piperidine-2-methylamine-L-arginine-L-alanine-acetate (Compound KC-13):

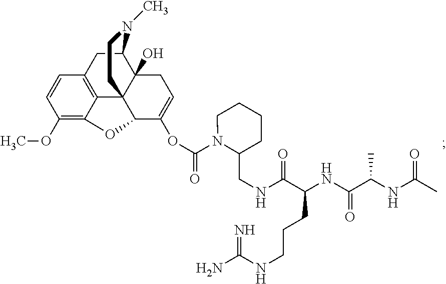

N-(oxycodone-6-enol-carbonyl)piperidine-2-methylamine-L-arginine-glycine-acetate (Compound KC-14):

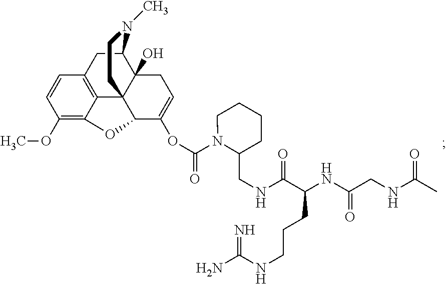

N-(oxycodone-6-enol-carbonyl)piperidine-2-methylamine-L-arginine-L-alanine-malonate (Compound KC-15):

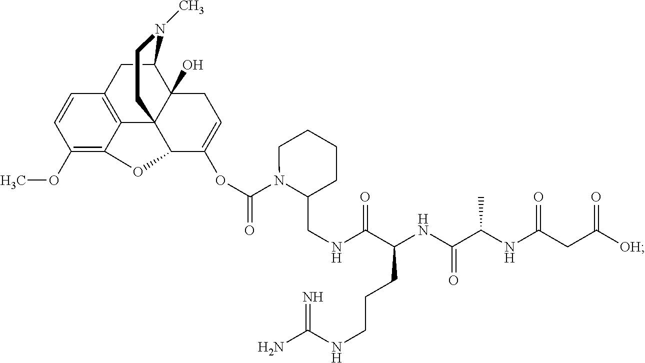

N-(oxycodone-6-enol-carbonyl)piperidine-2-methylamine-L-arginine-glycine-malonate (Compound KC-16):

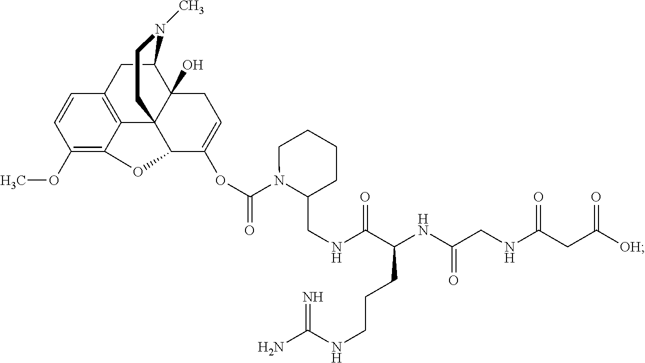

and

N-(hydrocodone-6-enol-carbonyl)-R-(piperidine-2-methylamine)-L-arginine-glycine-malonate (Compound KC-31):

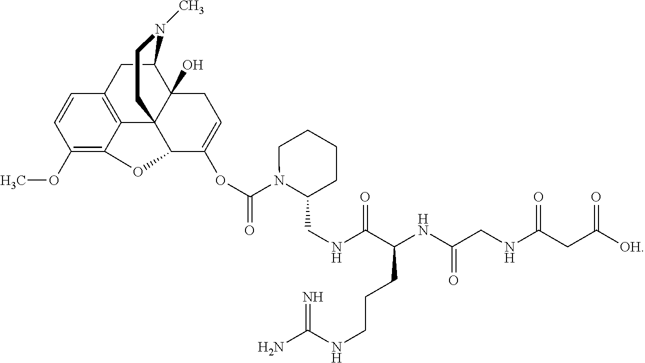

Particular compounds of interest, and salts or solvates or stereoisomers thereof, include:

Compound KC-32:

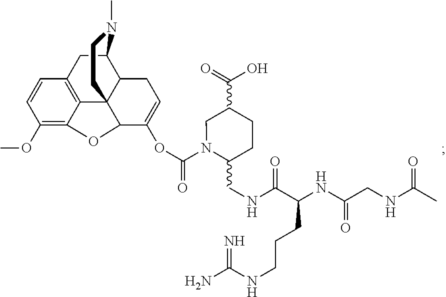

Compound KC-35:

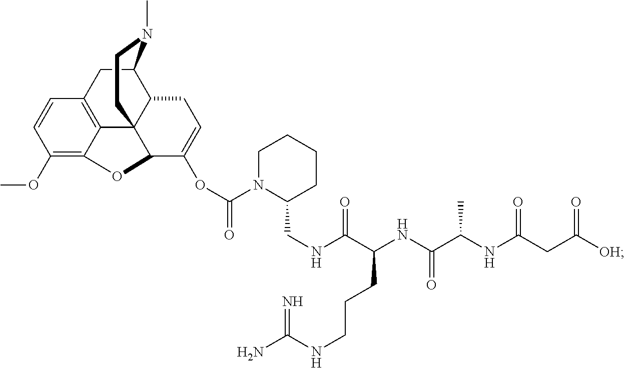

Compound KC-36:

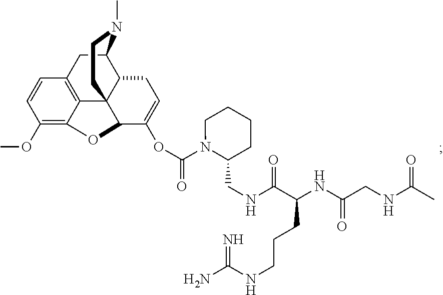

Compound KC-37:

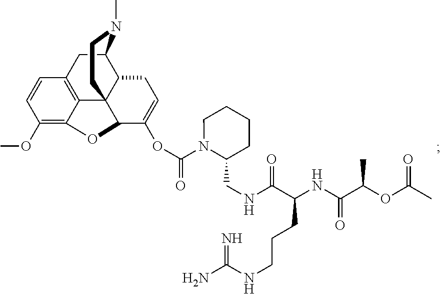

Compound KC-38:

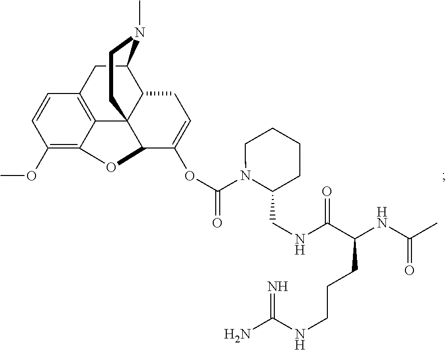

Compound KC-39:

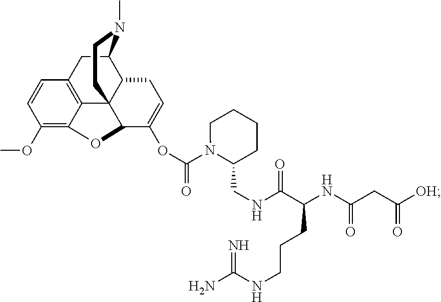

Compound KC-40:

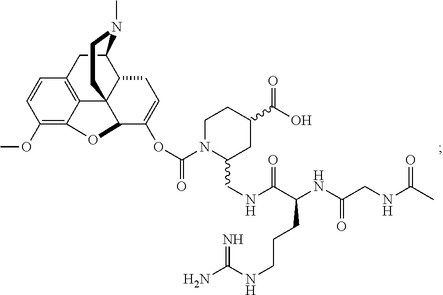

Compound KC-41:

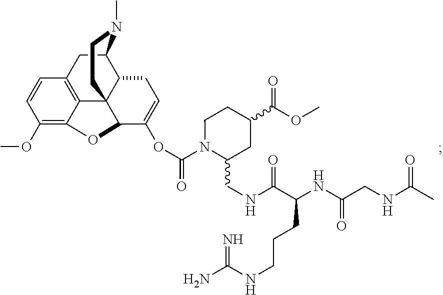

Compound KC-42:

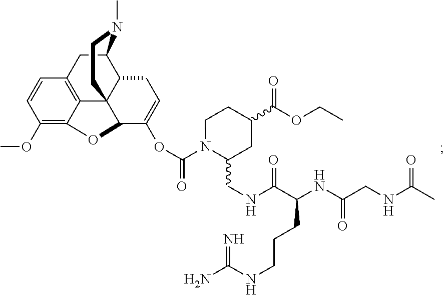

Compound KC-43:

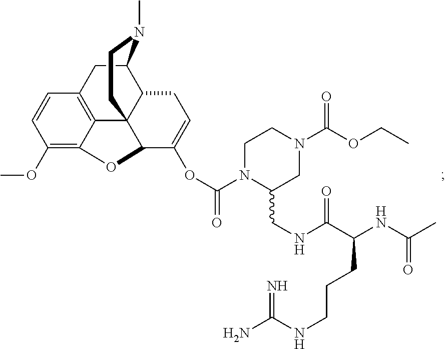

Compound KC-44:

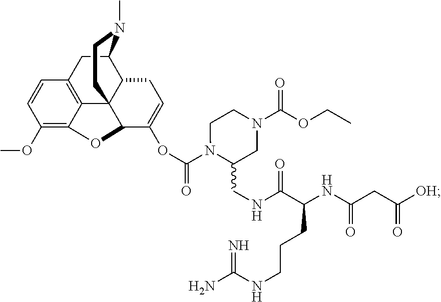

Compound KC-45:

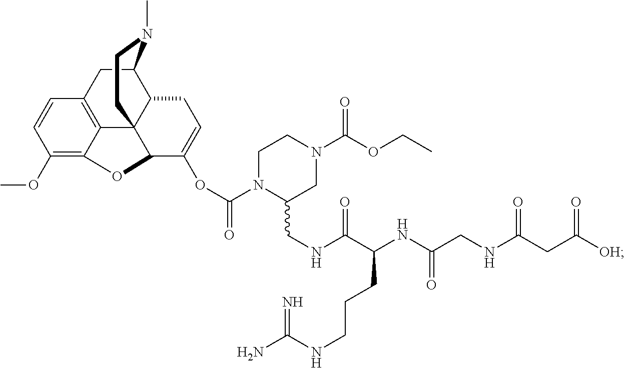

Compound KC-46:

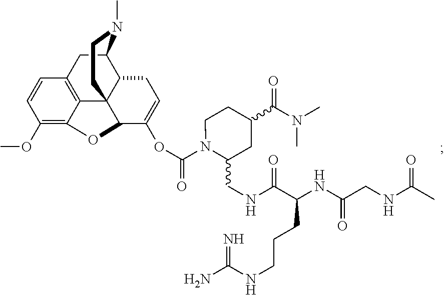

Compound KC-47:

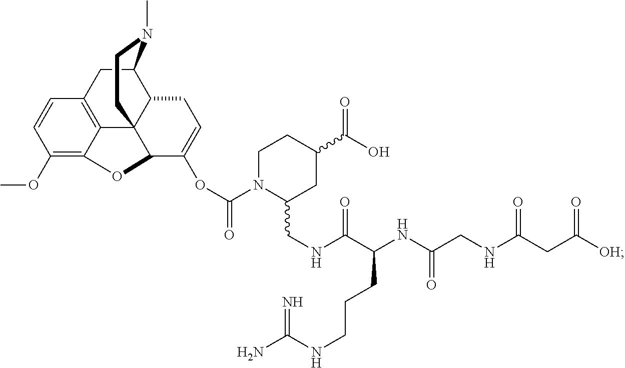

Compound KC-48:

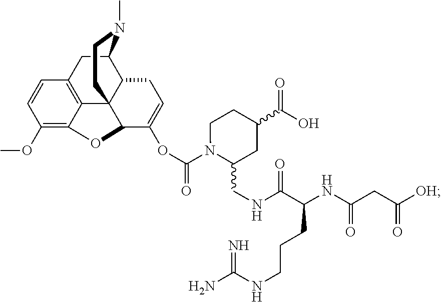

Compound KC-49:

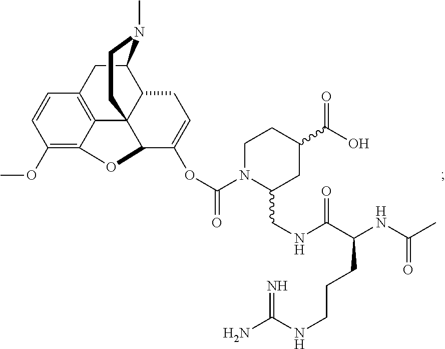

Compound KC-50:

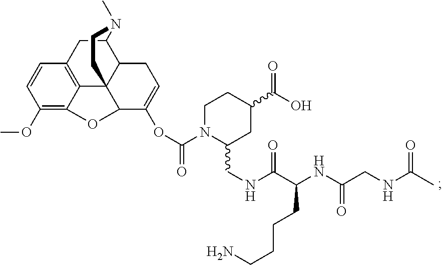

Compound KC-51:

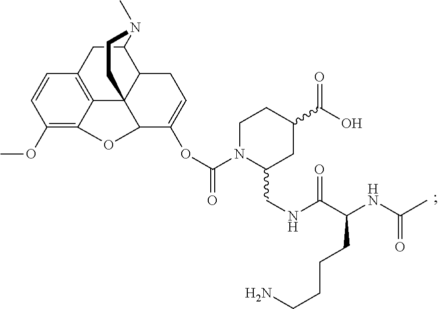

Compound KC-52:

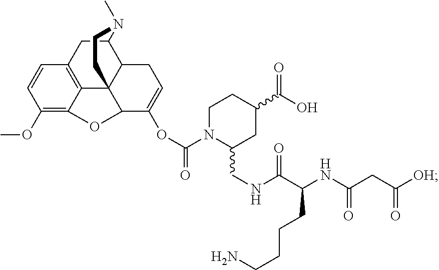

Compound KC-53:

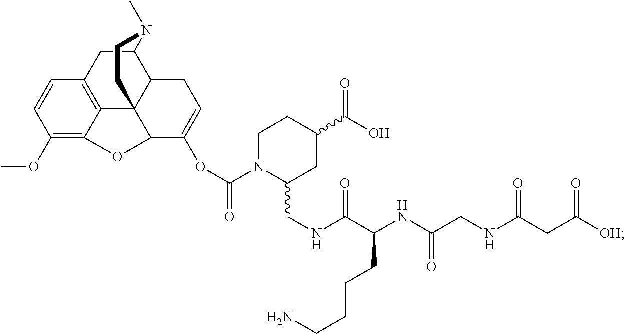

and

Compound KC-55:

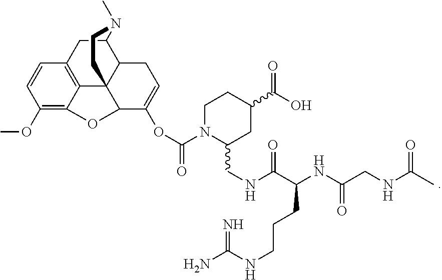

Phenolic Opioid Prodrugs with Heterocyclic Linkers

The disclosure provides a phenolic opioid prodrug that provides controlled release of a phenolic opioid. In a phenolic opioid prodrug, a promoiety is attached to the phenolic opioid through the phenolic oxygen atom. In a phenolic opioid prodrug, the oxygen atom of the phenol group of the phenolic opioid is replaced by a covalent bond to a promoiety.

As disclosed herein, an enzyme-cleavable phenolic opioid prodrug is a phenolic opioid prodrug that comprises a promoiety comprising an enzyme-cleavable moiety, i.e., a moiety having a site susceptible to cleavage by an enzyme. In one embodiment, the cleavable moiety is a GI enzyme-cleavable moiety, such as a trypsin-cleavable moiety. Such a prodrug comprises a phenolic opioid covalently bound to a promoiety comprising an enzyme-cleavable moiety, wherein cleavage of the enzyme-cleavable moiety by an enzyme mediates release of the drug.

Formula HP-(VI)

The present embodiments provide a compound of formula HP-(VI):

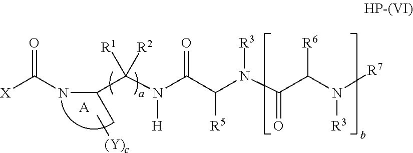

wherein

X represents a residue of a phenolic opioid, wherein the hydrogen atom of the phenolic hydroxyl group is replaced by a covalent bond to —C(O)—N[(A ring)-Y_{c}]-(CR¹R²)ₐ—NH—C(O)—CH(R⁵)—N(R³)—[C(O)—CH(R⁶)—N(R³)]_{b}—R⁷;

the A ring is a heterocyclic 5 to 12-membered ring;

each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;

c is a number from zero to 3;

each R′ is independently selected from hydrogen, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;

each R² is independently selected from hydrogen, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano; or

R¹ and R² together with the carbon to which they are attached can form a cycloalkyl or substituted cycloalkyl group, or two R′ or R² groups on adjacent carbon atoms, together with the carbon atoms to which they are attached, can form a cycloalkyl or substituted cycloalkyl group;

a is an integer from one to 8;

provided that when a is one, the A ring is a heterocyclic 6 to 12-membered ring; and when the A ring is a heterocyclic 5-membered ring, then a is an integer from 2 to 8;

each R³ is independently hydrogen, alkyl, substituted alkyl, aryl or substituted aryl;

R⁵ is selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl;

each R⁶ is independently selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl;

b is a number from zero to 100;

R⁷ is selected from hydrogen, alkyl, substituted alkyl, acyl, substituted acyl, alkoxycarbonyl, substituted alkoxycarbonyl, aryl, substituted aryl, arylalkyl, and substituted arylalkyl;

or a salt, hydrate or solvate thereof.

Particular compound of interest, and salts or solvates or stereoisomers thereof, includes:
N-(Tapentadol-carbonyl)piperidine-2-methylamine-L-arginine-malonate (Compound TP-5):

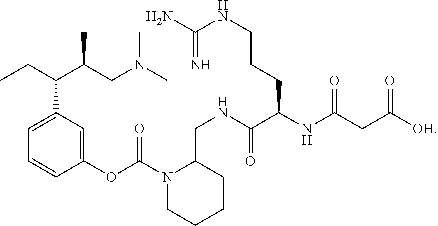

Amide-Modified Opioid Prodrugs with Heterocyclic Linkers

The disclosure provides an amide-modified opioid prodrug that provides controlled release of an amide-containing opioid. As shown below, in an amide-modified opioid prodrug, a promoiety is attached to the amide-containing opioid through the enolic oxygen atom of the amide enol moiety or through the oxygen of the imine tautomer. In an amide-modified opioid prodrug, the hydrogen atom of the corresponding enolic group of the amide enol or of the imine tautomer of the amide-containing opioid is replaced by a covalent bond to a promoiety. In certain embodiments, the promoiety that replaces the hydrogen atom of the corresponding enolic group of the amide enol or the imine tautomer of the amide-containing opioid contains an acyl group as the point of connection.

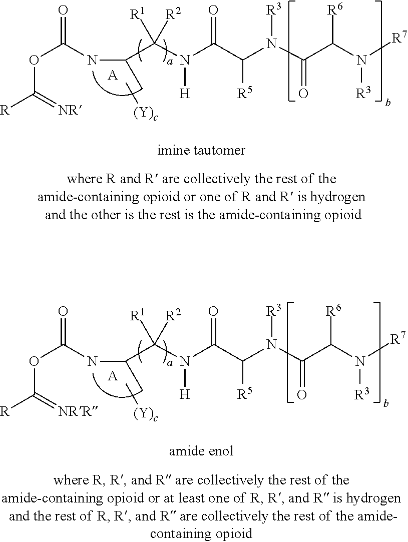

As disclosed herein, an enzyme-cleavable amide-modified opioid prodrug is an amide-modified opioid prodrug that comprises a promoiety comprising an enzyme-cleavable moiety, i.e., a moiety having a site susceptible to cleavage by an enzyme. Release of the opioid is mediated by enzymatic cleavage of the promoiety from the amide-containing opioid. In one embodiment, the cleavable moiety is a GI enzyme-cleavable moiety, such as a trypsin-cleavable moiety.

Formula HP-(X)

The present embodiments provide a compound of formula HP-(X):

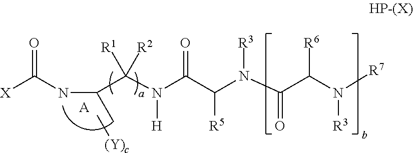

wherein

X represents a residue of an amide-containing opioid, wherein —C(O)—N[(A ring)-Y_{c}]-(CR¹R²)ₐ—NH—C(O)—CH(R⁵)—N(R³)—[C(O)—CH(R⁶)—N(R³)]_{b}—R⁷ is connected to the amide-containing opioid through the oxygen of the amide group, wherein the amide group is converted to an amide enol or an imine tautomer;

the A ring is a heterocyclic 5 to 12-membered ring;

each Y is independently selected from alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;

c is a number from zero to 3;

each R¹ is independently selected from hydrogen, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano;

each R² is independently selected from hydrogen, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, and cyano; or

R¹ and R² together with the carbon to which they are attached can form a cycloalkyl or substituted cycloalkyl group, or two R¹ or R² groups on adjacent carbon atoms, together with the carbon atoms to which they are attached, can form a cycloalkyl or substituted cycloalkyl group;

a is an integer from one to 8;

provided that when a is one, the A ring is a heterocyclic 6 to 12-membered ring; and when the A ring is a heterocyclic 5-membered ring, then a is an integer from 2 to 8;

each R³ is independently hydrogen, alkyl, substituted alkyl, aryl or substituted aryl;

R⁵ is selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl;

each R⁶ is independently selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl;

b is a number from zero to 100;

R⁷ is selected from hydrogen, alkyl, substituted alkyl, acyl, substituted acyl, alkoxycarbonyl, substituted alkoxycarbonyl, aryl, substituted aryl, arylalkyl, and substituted arylalkyl;

or a salt, hydrate or solvate thereof.

General Synthetic Procedures for Opioid Prodrugs with Heterocyclic Linkers

A representative synthesis for Compound S-104 is shown in Scheme HP-1. In Scheme HP-1, R^{a}, A ring, Y, and c are defined herein. PG¹ is an amino protecting group.

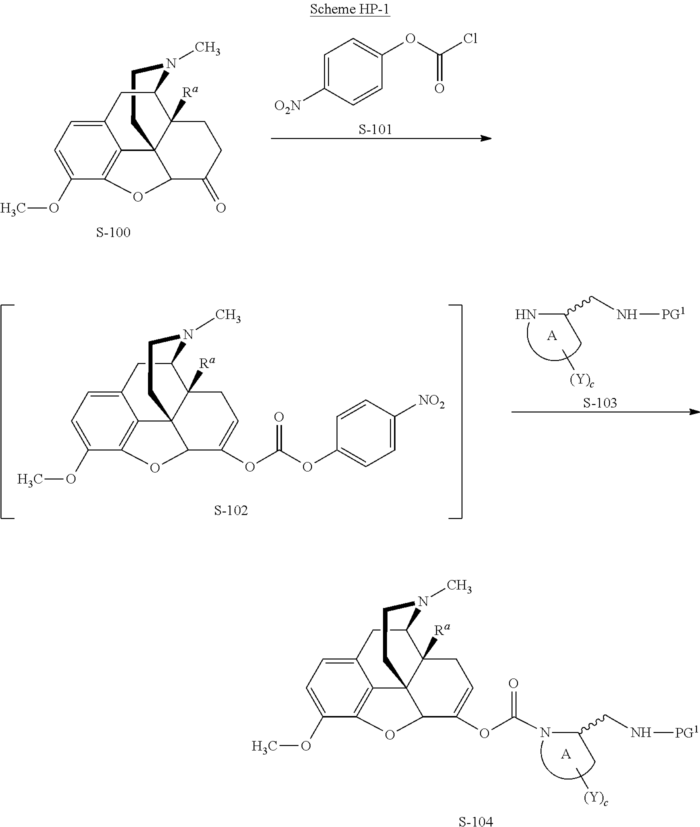

In Scheme HP-1, Compound S-100 is a commercially available starting material. Alternatively, Compound S-100 can be semi-synthetically derived from natural materials or synthesized via a variety of different synthetic routes using commercially available starting materials and/or starting materials prepared by conventional synthetic methods.

With continued reference to Scheme HP-1, Compound S-100 is enolized. Enolization of a ketone can be performed with reaction with a strong base, such as potassium hexamethyldisilazide (KHMDS). The enolate of Compound S-100 is then reacted with an activation agent, such as Compound S-101, to form intermediate Compound S-102. Suitable activation agents include carbonate-forming reagents, such as chloroformates. In Scheme 1, the activation agent Compound S-101 is 4-nitrophenyl chloroformate. Other suitable activation agents can be used prior to reaction with Compound S-103.

With continued reference to Scheme HP-1, Compound S-102 reacts with Compound S-103 to form Compound S-104. In Scheme 1, Compound S-103 is a commercially available starting material. Alternatively, Compound S-103 can be synthesized via a variety of different synthetic routes using commercially available starting materials and/or starting materials prepared by conventional synthetic methods.

A representative synthesis for Compound S-203 is shown in Scheme HP-2. In Scheme HP-2, R^{a}, A ring, Y, c, and R⁵ are defined herein. PG¹ and PG² are amino protecting groups.

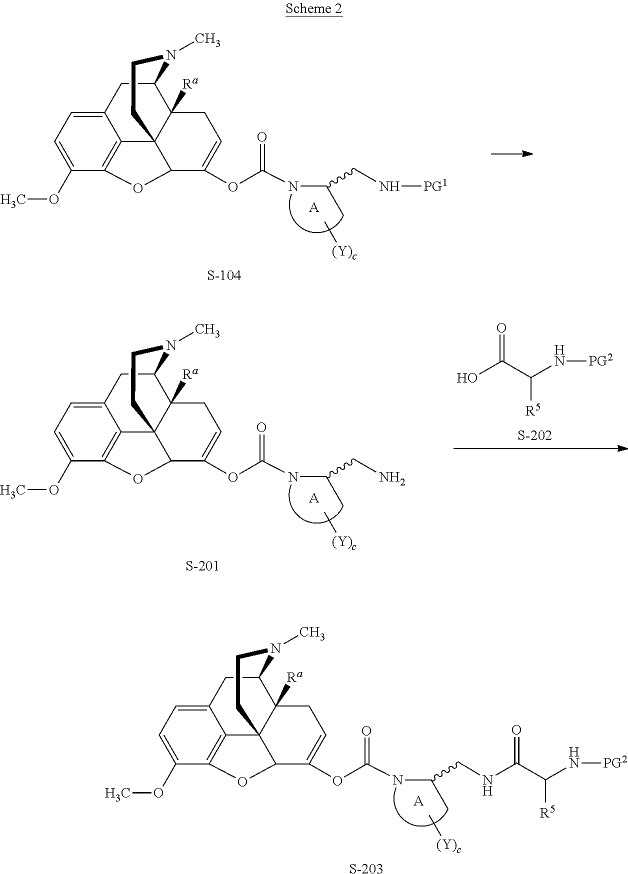

In Scheme HP-2, the protecting group PG¹ is removed from Compound S-104 to form Compound S-201. Conditions to remove amino groups can be found in Greene and Wuts. When PG¹ is a Boc group, the protecting group can be removed with acidic conditions, such as treatment with hydrochloric acid or trifluoroacetic acid.

With reference to Scheme HP-2, Compound S-201 reacts with Compound S-202 to form Compound S-203 in a peptide coupling reaction. In certain embodiments, R⁵ is a side chain of an amino acid and is optionally protected. Protecting groups for the side chain of amino acids are known to those skilled in art and can be found in Greene and Wuts. In certain instances, the protecting group for the side chain of arginine is a sulfonyl-type protecting group, such as 2,2,4,6,7-pentamethyldihydrobenzofurane (Pbf). Other protecting groups include 2,2,5,7,8-pentamethylchroman (Pmc) and 1,2-dimethylindole-3-sulfonyl (MIS).

A peptide coupling reaction typically employs a conventional peptide coupling reagent and is conducted under conventional coupling reaction conditions, typically in the presence of a trialkylamine, such as triethylamine or diisopropylethylamine (DIEA). Suitable coupling reagents for use include, by way of example, carbodiimides, such as ethyl-3-(3-dimethylamino)propylcarbodiimide (EDC), dicyclohexylcarbodiimide (DCC), diisopropylcarbodiimide (DIC) and the like, and other well-known coupling reagents, such as N,N′-carbonyldiimidazole, 2-ethoxy-1-ethoxycarbonyl-1,2-dihydroquinoline (EEDQ), benzotriazol-1-yloxy-tris(dimethylamino)phosphonium hexafluorophosphate (BOP), O-(7-azabenzotriazol-1-yl)-N,N,N,N′,N′-tetramethyluronium hexafluorophosphate (HATU) and the like. Optionally, well-known coupling promoters, such as N-hydroxysuccinimide, 1-hydroxybenzotriazole (HOBT), 1-hydroxy-7-azabenzotriazole (HOAT), N,N-dimethylaminopyridine (DMAP) and the like, can be employed in this reaction. Typically, this coupling reaction is conducted at a temperature ranging from about 0° C. to about 60° C. for about 1 to about 72 hours in an inert diluent, such as THF or DMF. In certain instances, Compound S-201 reacts with Compound S-202 to form Compound S-203 in the presence of HATU.

A representative synthesis for Compound S-303 is shown in Scheme HP-3. In Scheme HP-3, R^{a}, A ring, Y, c, R⁵, R⁶, and R⁷ are defined herein. PG² is an amino protecting group.

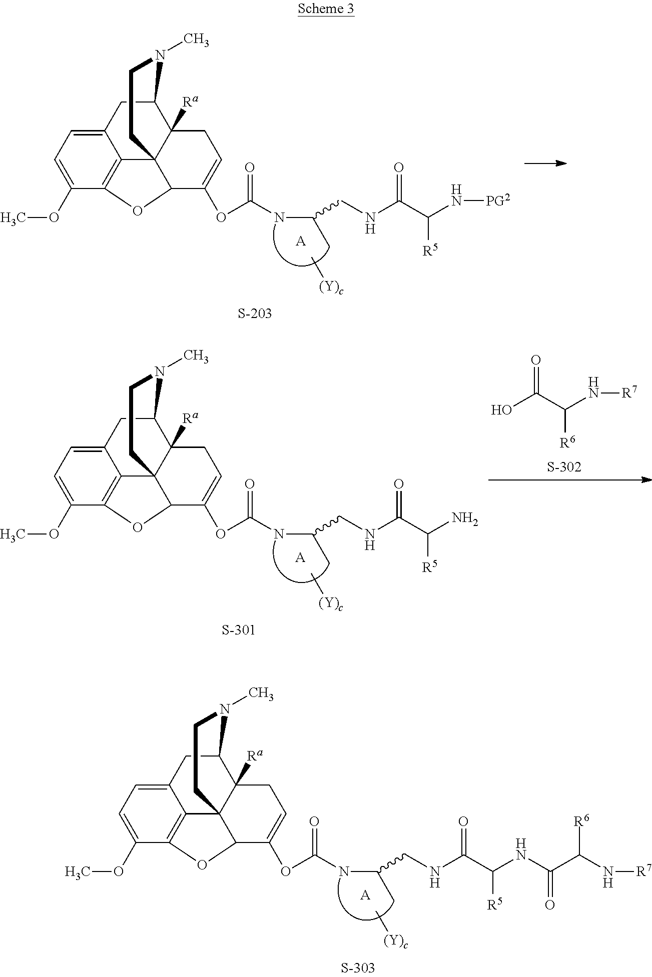

In Scheme HP-3, the protecting group PG² is removed from Compound S-203 to form Compound S-301. Conditions to remove amino groups can be found in Greene and Wuts. When PG² is a Boc group, the protecting group can be removed with acidic conditions, such as treatment with hydrochloric acid or trifluoroacetic acid.

With reference to Scheme HP-3, Compound S-301 reacts with Compound S-302 to form Compound S-303 in a peptide coupling reaction. A peptide coupling reaction typically employs a conventional peptide coupling reagent and is conducted under conventional coupling reaction conditions, typically in the presence of a trialkylamine, such as triethylamine or diisopropylethylamine (DIEA). Suitable coupling reagents for use include, by way of example, carbodiimides, such as ethyl-3-(3-dimethylamino)propylcarbodiimide (EDC), dicyclohexylcarbodiimide (DCC), diisopropylcarbodiimide (DIC) and the like, and other well-known coupling reagents, such as N,N′-carbonyldiimidazole, 2-ethoxy-1-ethoxycarbonyl-1,2-dihydroquinoline (EEDQ), benzotriazol-1-yloxy-tris(dimethylamino)phosphonium hexafluorophosphate (BOP), O-(7-azabenzotriazol-1-yl)-N,N,N,N′,N′-tetramethyluronium hexafluorophosphate (HATU) and the like. Optionally, well-known coupling promoters, such as N-hydroxysuccinimide, 1-hydroxybenzotriazole (HOBT), 1-hydroxy-7-azabenzotriazole (HOAT), N,N-dimethylaminopyridine (DMAP) and the like, can be employed in this reaction. Typically, this coupling reaction is conducted at a temperature ranging from about 0° C. to about 60° C. for about 1 to about 72 hours in an inert diluent, such as THF or DMF. In certain instances, Compound S-301 reacts with Compound S-302 to form Compound S-303 in the presence of HATU.

In certain instances in Scheme HP-3, Compound S-301 is reacted with Compound S-302 with R⁷ as a protecting group for an amino group. In these instances, the protecting group can be removed and the R⁷ group as an N-derivative group can be attached. Conditions for removal of other protecting groups depend on the identity of the protecting group and are known to those skilled in the art. The conditions can also be found in Greene and Wuts. For example, a malonyl group can be attached via a reaction with mono-tert-butyl malonate. Reaction using mono-tert-butyl malonate can be aided with use of activation reagents, such as symmetric anhydrides, O-(benzotriazol-1-yl)-N,N,N′,N′-tetramethyluronium hexafluorophosphate (HBTU), dicyclohexylcarbodiimide (DCC) diisopropylcarbodiimide (DIC)/1-hydroxybenzotriazole (HOBt), and benzotriazole-1-yl-oxytris(dimethylamino)phosphonium hexafluorophosphate (BOP). In another example, an alkanoyl group, such as an acetyl group, can be attached via a reaction with alkanoyl anhydride or alkanoyl halide.

Additional amino acids can be added to the compound through standard peptide coupling reactions as discussed herein. Removal of other protecting groups can be performed if other protecting groups were used, such as protecting groups present on the R⁵ or R⁶ moiety. Conditions for removal of other protecting groups depend on the identity of the protecting group and are known to those skilled in the art. The conditions can also be found in Greene and Wuts.

Amphetamine Prodrugs

Amphetamine refers to a chemical substance that exerts its pharmacological action by modulating neurotransmitters, such as dopamine, serotonin and norepinephrine. In certain embodiments, amphetamine is a compound with a pharmacophore that crosses the blood-brain barrier and has CNS stimulation and central appetite suppressant effects. See, for example, Foye's Principles of Medicinal Chemistry, Sixth Edition, ed. T. L. Lemke and D. A. Williams, Lippincott Williams & Wilkins, 2008, particularly Chapter 13, pages 392-416.

The present disclosure provides an amphetamine prodrug which provides enzymatically-controlled release of amphetamine. The disclosure provides a promoiety that is attached to amphetamine through the amphetamine amino group.

“Amino-containing amphetamine analogs” or amphetamine analogs” refer to analogs or derivatives of amphetamine that contain an amino group. For instance, the following amphetamine analogs contain an amino group that can be a point of attachment to a promoiety through the amino group: amphetamine (i.e., 1-phenylpropan-2-amine), Benzedrine (i.e., dl-amphetamine), dextroamphetamine (i.e., d-amphetamine), levoamphetamine (i.e., l-amphetamine), 4-fluoroamphetamine (4-FA), 3-fluoroamphetamine (3-FA), 2-fluoroamphetamine (2-FA), 4-methylthioamphetamine (4-MTA), 3,4-methylenedioxyamphetamine (MDA), para-methoxyamphetamine (PMA), 3-methoxyamphetamine (3-MeOA), 4-ethoxyamphetamine (4-ETA), 2,5-dimethoxy-4-ethoxyamphetamine (MEM), 2,5-dimethoxy-4-propoxyamphetamine (MPM), 4-methylamphetamine (4-MA), 2-methylamphetamine (2-MA), 3-methylamphetamine (3-MA), 3,4-dimethylamphetamine, 3-methoxy-4-methylamphetamine (MMA), 3-trifluoromethylamphetamine, 3-hydroxyamphetamine, 4-hydroxyamphetamine, (1R,2S)-3-[-2-amino-1-hydroxy-propyl]phenol, 2,5-dimethoxy-4-methylamphetamine (DOM), 2,6-dimethoxy-4-methylamphetamine (Ψ-DOM), indanylamphetamine, 5-(2-aminopropyl)-2,3-dihydrobenzofuran (5-APDB), 6-(2-aminopropyl)-2,3-dihydrobenzofuran (6-APDB), 5-(2-aminopropyl)indole (5-IT), naphthylaminopropane (NAP), phenylpropanolamine (PPA), d-norpseudoephedrine, benzoylethanamine, para-bromoamphetamine (PBA), para-chloroamphetamine (PCA), para-iodoamphetamine (PIA), α,β-dimethylamphetamine, o-chloro-α,α-dimethylphenethylamine, 3,4-dihydroxyamphetamine (3,4-DHA), 2,4-dimethoxyamphetamine (2,4-DMA), 2,5-dimethoxyamphetamine (2,5-DMA), 3,4-dimethoxyamphetamine (3,4-DMA), α-methylnorepinephrine (α-Me-NE), 2,5-dimethoxy-4-methylthioamphetamine (Aleph), 2,5-dimethoxy-4-ethylthioamphetamine (Aleph-2), 2,5-dimethoxy-4-isopropylthioamphetamine (Aleph-4), 2,5-dimethoxy-4-phenylthioamphetamine (Aleph-6), 2,5-dimethoxy-4-propylthioamphetamine (Aleph-7), 2,5-dimethoxybromoamphetamine (DOB), 2,5-dimethoxychloroamphetamine (DOC), 2,5-dimethoxyfluoroethylamphetamine (DOEF) 2,5-dimethoxyethylamphetamine (DOET), 2,5-dimethoxyfluoroamphetamine (DOF), 2,5-dimethoxyiodoamphetamine (DOI), 2,5-dimethoxynitroamphetamine (DON), 2,5-dimethoxypropylamphetamine (DOPR), 2,5-dimethoxytrifluoromethylamphetamine (DOTFM), 2-methyl-3,4-methylenedioxyamphetamine (2-methyl-MDA), 3-methyl-4,5-methylenedioxyamphetamine (5-methyl-MDA), 3-methoxy-4,5-methylenedioxyamphetamine (MMDA), 2-methoxy-4,5-methylenedioxyamphetamine (MMDA-2), 2-methoxy-3,4-methylenedioxyamphetamine (MMDA-3a), 4-methoxy-2,3-methylenedioxyamphetamine (MMDA-3b), 2-methylthio-3,4-methylenethioxyamphetamine (2T-MMDA-3a), 2-methoxy-4,5-methylenethioxyamphetamine (4T-MMDA-2), 3,4,5-trimethoxyamphetamine (TMA), 2,4,5-trimethoxyamphetamine (TMA-2), 2,3,4-trimethoxyamphetamine (TMA-3), 2,3,5-trimethoxyamphetamine (TMA-4), 2,3,6-trimethoxyamphetamine (TMA-5), 2,4,6-trimethoxyamphetamine (TMA-6), 2,5-dimethoxy-3,4-dimethylamphetamine, 2,5-dimethoxy-3,4-methylenedioxyamphetamine (DMMDA), tyramine, phentermine, alpha-allyl-phenethylamine, (1-(8-bromobenzo[1,2-b; 4,5-b]difuran-4-yl)-2-aminopropane (bromo-DragonFLY), 3,4,5-trimethoxyphenethylamine (mescaline), 2,5-dimethoxy-4-bromophenethylamine (2C-B), 2,5-dimethoxy-4-chlorophenethylamine (2C-C), 2,5-dimethoxy-4-iodophenethylamine (2C-I), 2,5-dimethoxy-4-methyl-phenethylamine (2C-D), 2,5-dimethoxy-4-ethylphenethylamine (2C-E), 2,5-dimethoxy-4-n-propylphenethylamine (2C-P), 2,5-dimethoxy-4-fluorophenethylamine (2C-F), 2,5-dimethoxy-4-nitrophenethylamine (2C-N), 2,5-dimethoxy-4-ethylthio-phenethylamine (2C-T-2), 2,5-dimethoxy-4-isopropylthio-phenethylamine (2C-T-4), 2,5-dimethoxy-4-propylthio-phenethylamine (2C-T-7), 2,5-dimethoxy-4-cyclopropylmethylthio-phenethylamine (2C-T-8), 2,5-dimethoxy-4-tert-butylthio-phenethylamine (2C-T-9), 2,5-dimethoxy-4-(2-fluoroethylthio)-phenethylamine (2C-T-21), ephedrine, pseudoephedrine, and the like.

Any type of reactive group on an amphetamine analog can provide a handle for a point of attachment to a promoiety. Examples of reactive groups on an amphetamine analog include, but are not limited to, amino, amide, alcohol (including phenol), and ketone. In certain embodiments, an amino group on an amphetamine analog provides a point of attachment to a promoiety by reaction to form an amino linkage or an amide. For example, the amino group of the amphetamine analog can provide a point of attachment to a promoiety by reaction to form an amino linkage or an amide. An amide on an amphetamine analog can provide a point of attachment to a promoiety by reaction to form a linkage, such as an amide enol or an N-acylated amide. An alcohol (e.g., phenol) on an amphetamine analog can provide a point of attachment to a promoiety by reaction to form a linkage, such as a carbamate, a carbonate, an ether, or an ester. A ketone on an amphetamine analog can provide a point of attachment to a promoiety by reaction to form a linkage, such as an enol carbamate.

It is contemplated that amphetamine analogs bearing at least some of the functionalities described herein will be developed; such amphetamines are included as part of the scope of this disclosure.

The disclosure provides for an amphetamine prodrug, wherein amphetamine or the amphetamine analog has an optionally substituted amphetamine residue of the following general structure:

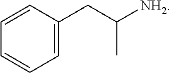

In certain embodiments, a promoiety can be attached to amphetamine or the amphetamine analog via modification of the amino moiety of the amphetamine residue. Release of amphetamine or the amphetamine analog is mediated by enzymatic cleavage of the promoiety from amphetamine or the amphetamine analog. In certain embodiments, a promoiety can be attached to amphetamine through the amino moiety of the amphetamine residue, such as via a covalent bond. Release of amphetamine or the amphetamine analog is mediated by enzymatic cleavage of the promoiety from amphetamine or the amphetamine analog. In some cases, the promoiety comprises a trypsin-cleavable moiety that is susceptible to cleavage by trypsin. Such cleavage can initiate, contribute to or effect drug release.

The disclosure provides an amphetamine prodrug which provides enzymatically-controlled release of amphetamine or an amphetamine analog. In an amphetamine prodrug, a promoiety is attached via modification of the amino moiety of the amphetamine residue, such as through an amino linkage or as an amide. Release of amphetamine or the amphetamine analog is mediated by enzymatic cleavage of the promoiety from amphetamine or the amphetamine analog. The disclosure provides for release of amphetamine or the amphetamine analog through enzyme cleavage of the promoiety from amphetamine or the amphetamine analog.

Formula AM-(I)

The disclosure provides compounds of the general formula AM-(I):

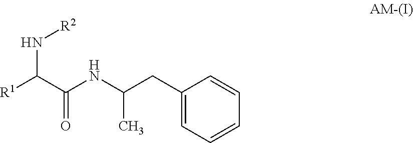

wherein

R¹ is selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl; and

R² is an acyl, substituted acyl, or an N-acyl derivative of a peptide;

or a salt, hydrate or solvate thereof.

The disclosure provides for a compound of the following formula:

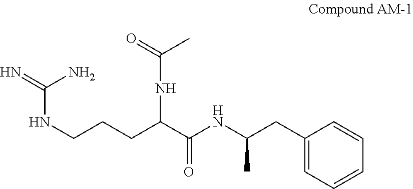

or a salt, hydrate or solvate thereof.

The disclosure provides for a compound of the following formula:

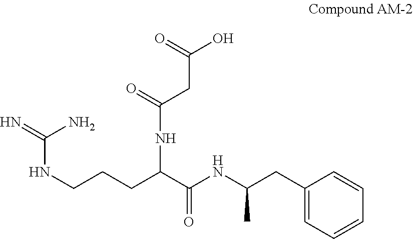

or a salt, hydrate or solvate thereof.

General Synthetic Procedures for Amphetamine Prodrugs

The compounds described herein may be obtained via the routes generically illustrated in Scheme AM-1.

The promoieties described herein, may be prepared and attached to compounds containing amino groups by procedures known to those of skill in the art (See e.g., Green et al., “Protective Groups in Organic Chemistry,” (Wiley, 2^{nd} ed. 1991); Harrison et al., “Compendium of Synthetic Organic Methods,” Vols. 1-8 (John Wiley and Sons, 1971-1996); “Beilstein Handbook of Organic Chemistry,” Beilstein Institute of Organic Chemistry, Frankfurt, Germany; Feiser et al., “Reagents for Organic Synthesis,” Volumes 1-17, (Wiley Interscience); Trost et al., “Comprehensive Organic Synthesis,” (Pergamon Press, 1991); “Theilheimer's Synthetic Methods of Organic Chemistry,” Volumes 1-45, (Karger, 1991); March, “Advanced Organic Chemistry,” (Wiley Interscience), 1991; Larock “Comprehensive Organic Transformations,” (VCH Publishers, 1989); Paquette, “Encyclopedia of Reagents for Organic Synthesis,” (John Wiley & Sons, 1995), Bodanzsky, “Principles of Peptide Synthesis,” (Springer Verlag, 1984); Bodanzsky, “Practice of Peptide Synthesis,” (Springer Verlag, 1984). Further, starting materials may be obtained from commercial sources or via well established synthetic procedures, supra.

Compounds AM-1 and AM-2 may be obtained via the routes generically illustrated in Scheme AM-1.

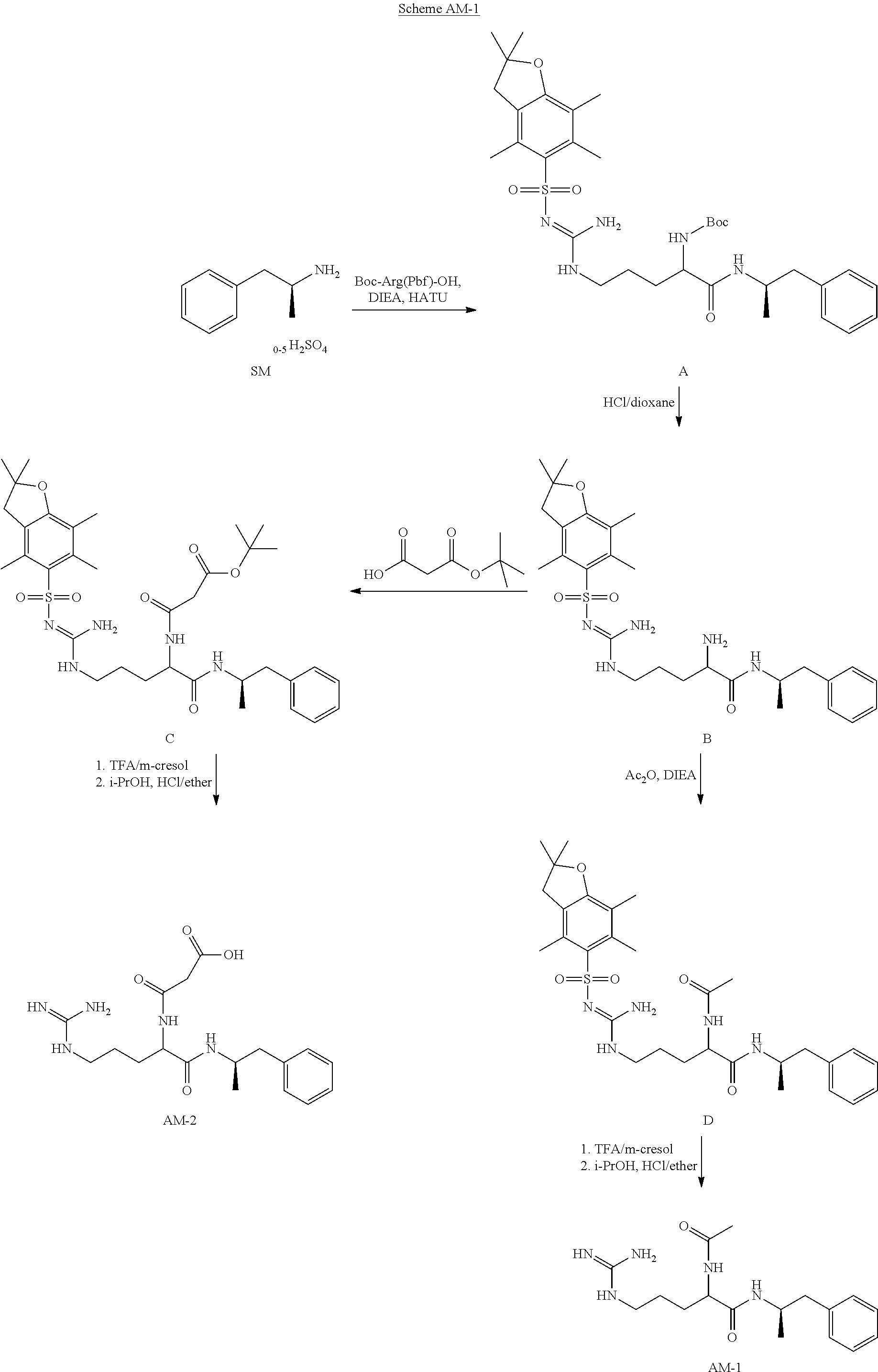

In Scheme AM-1, Compound SM is coupled with Boc-Arg(Pbf)-OH to form Compound A. Standard peptide coupling reagents can be used for the reaction. Suitable peptide coupling reagents include, but are not limited to, EDCI and HOBt, PyBroP and diisopropylethylamine, or HATU. Then, the Boc group of Compound A is removed to yield Compound B. The Boc group can be removed with acidic conditions. Suitable reagents that can be used for the deprotection reaction include trifluoroacetic acid and hydrochloric acid.

With further reference to Scheme AM-1, a malonyl group is attached to Compound B via a reaction with mono-tert-butyl malonate to form Compound C. Reaction between Compound B and mono-tert-butyl malonate can be aided with use of activation reagents, such as symmetric anhydrides, O-(benzotriazol-1-yl)-N,N,N′,N′-tetramethyluronium hexafluorophosphate (HBTU), dicyclohexylcarbodiimide (DCC) diisopropylcarbodiimide (DIC)/1-hydroxybenzotriazole (HOBt), and benzotriazole-1-yl-oxytris(dimethylamino)phosphonium hexafluorophosphate (BOP).

Then, the Pbf group of Compound C is removed to yield Compound AM-2. The Pbf group and can be removed with acidic conditions A suitable reagent that can be used for the deprotection reaction is trifluoroacetic acid.

In another synthetic route to obtain Compound AM-1, Compound B is acetylated at the amino group to yield Compound D. Acetylation of amino groups can be performed with acetic anhydride, acetic acid, or an acetyl halide.

Then, the Pbf group of Compound D is removed to yield Compound AM-1. The Pbf group and can be removed with acidic conditions. A suitable reagent that can be used for the deprotection reaction is trifluoroacetic acid.

Other Prodrugs that Release Second Drugs

The disclosure also provides any GI enzyme-cleavable prodrug that releases a second drug, in addition to those disclosed herein, such as other phenol-modified second drug prodrugs, other alcohol-modified second drug prodrugs, other ketone-modified second drug prodrugs, other amino-modified second drug prodrugs, and other amide-modified second drug prodrugs. The disclosure also provides any GI enzyme-cleavable opioid prodrug in addition to those disclosed herein, such as other phenol-modified opioid prodrugs, other alcohol-modified opioid prodrugs, other ketone-modified opioid prodrugs, amino-modified opioid prodrugs, and amide-modified opioid prodrugs. The disclosure also provides any other GI enzyme-cleavable amphetamine prodrugs.

### Amino Acids Found in Prodrugs

“Amino acid” means a building block of a polypeptide. As used herein, “amino acid” includes the 20 common naturally occurring L-amino acids and all amino acids variants. In certain embodiments, an amino acid is a cleavable substrate for a gastrointestinal enzyme.

“Naturally occurring amino acids” means the 20 common naturally occurring L-amino acids, that is, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine.

“Amino acid variants” means an amino acid other than any of the 20 common naturally occurring L-amino acids that is hydrolysable by a protease in a manner similar to the ability of a protease to hydrolyze a naturally occurring L-amino acid. Amino acid variants, thus, include amino acids or analogs of amino acids other than the 20 naturally-occurring amino acids. Amino acid variants include synthetic amino acids. Amino acid variants also include amino acid derivatives. A derivative refers to a substance that has been altered from another substance by modification, partial substitution, homologation, truncation, or a change in oxidation state while retaining the ability to be cleaved by a GI enzyme.

Certain examples of amino acid variants include, but are not limited to: 2-aminoindane-2-carboxylic acid, 2-aminoisobutyric acid, 4-amino-phenylalanine, 5-hydroxylysine, biphenylalanine, citrulline, cyclohexylalanine, cyclohexylglycine, diethylglycine, dipropylglycine, homoarginine, homocitrulline, homophenylalanine, homoproline, homoserine, homotyrosine, hydroxyproline, lanthionine, naphthylalanine, norleucine, ornithine, phenylalanine(4-fluoro), phenylalanine(4-nitro), phenylglycine, pipecolic acid, tert-butylalanine, tert-butylglycine, tert-leucine, tetrahydroisoquinoline-3-carboxylic acid, α-aminobutyric acid, γ-amino butyric acid, 2,3-diaminoproprionic acid, phenylalanine(2,3,4,5,6 pentafluoro), aminohexanoic acid and derivatives thereof.

Certain examples of amino acid variants include, but are not limited to, N-methyl amino acids. For example, N-methyl-alanine, N-methyl aspartic acid, N-methyl-glutamic acid, N-methyl-glycine (sarcosine) are N-methyl amino acids.

Certain examples of amino acid variants include, but are not limited to: dehydroalanine, ethionine, hypusine, lanthionine, pyrrolysine, α-aminoisobutyric acid, selenomethionine and derivatives thereof.

Certain examples of amino acid variants include, but are not limited to: (3,2-amino benzoic acid, 2-amino methyl benzoic acid, 2-amino-3-guanidinopropionic acid, 2-amino-3-methoxy benzoic acid, 2-amino-3-ureidopropionic acid, 3-amino benzoic acid, 4-amino benzoic acid, 4-amino methyl benzoic acid, 4-nitroanthranillic acid, 5-acetamido-2-aminobenzoic acid, butanoic acid (HMB), glutathione, homocysteine, statine, taurine, β-alanine, 2-hydroxy-4-(methylthio), (3,4)-diamino benzoic acid, (3,5)-diamino benzoic acid and derivatives thereof.

Certain examples of amino acid variants include, but are not limited to: (2 amino ethyl) cysteine, 2-amino-3-ethyoxybutanoic acid, buthionine, cystathion, cysteic acid, ethionine, ethoxytheorine, methylserine, N-ε-ε-dimethyl-lysine, N-ω-nitro-arginine, saccharopine, isoserine derivatives thereof, and combinations thereof.

Certain examples of amino acid variants include, but are not limited to: l-carnitine, selenocysteine, l-sarcosine, l-lysinol, benzoic acid, citric acid, choline, EDTA or succinic acid and derivatives thereof.

Certain examples of amino acid variants are amino alcohols. Examples of amino alcohols include, but are not limited to: alaminol, indano, norephedrine, asparaginol, aspartimol, glutamol, leucinol, methioninol, phenylalaminol, prolinol, tryptophanol, valinol, isoleucinol, argininol, serinol, tyrosinol, threoninol, cysteinol, lysinol, histidinol and derivatives thereof.

### Pharmaceutical Compositions and Methods of Use in Adverse Side Effect Risk Reduction

The present disclosure provides pharmaceutical compositions of a first drug and a GI-enzyme inhibitor, where the first drug is one that poses of risk of an adverse drug-drug interaction due to an additive or synergistic effect of the first drug and a second drug, where the second drug is co-ingested as a GI-enzyme cleavable prodrug ingested by a patient. Such pharmaceutical compositions comprising such a first drug and a GI enzyme inhibitor are referred to herein as “first pharmaceutical compositions.”

In one non-limiting example, the first pharmaceutical composition comprises a GABA_{A} agonist (e.g., benzodiazapene or other GABA_{A} agonist exemplified above) and a GI enzyme inhibitor (e.g., a trypsin inhibitor), where such first pharmaceutical composition is adapted for reduction of risk of adverse side effects in a patient who is at risk of co-ingesting a GI-enzyme cleavable opioid prodrug.

In one non-limiting example, the first pharmaceutical composition comprises a monoamine oxidase inhibitor (MAUI) and a GI enzyme inhibitor (e.g., a trypsin inhibitor), where such first pharmaceutical composition is for reduction of risk of adverse side effects in a patient who is at risk of co-ingesting, or who has ingested, a GI-enzyme cleavable amphetamine prodrug.

In other non-limiting examples, the first pharmaceutical composition comprises a GI enzyme inhibitor (e.g., trypsin inhibitor) and a first drug, wherein the first drug is an adrenergic receptor antagonist, an NMDA receptor antagonist, a CNS depressant, or a drug that can cause serotonin syndrome, with specific examples of such drugs provided above. The second drug can be one administrable as a GI enzyme-cleavable prodrug, where the released drug is an opioid, an amphetamine, or an amphetamine analog. Examples of GI enzyme-cleavable prodrugs are provided in detail above.

Examples of adverse drug-drug interactions that can follow co-ingestion of a first drug and a second drug as described above can include respiratory depression, hypoventilation, hypertension, and serotonin syndrome, wherein some adverse side effects can result in permanent damage, and can lead to death. Risk of such adverse side effects may be particularly high in situations where a patient ingests both the first drug and the second drug prior to a decrease in physical activity, e.g., prior to lying down to rest, e.g., as in prior to bedtime. For example, risk of respiratory depression following ingestion of a sedative (e.g., a GABA_{A} agonist) as a first drug and a GI-enzyme cleavable prodrug of an opioid can be increased if such are ingested within a short period (e.g., 1 hour, 2 hours) prior to rest.

The present disclosure provides methods of reducing risk of adverse side effects due to drug-drug interaction by administering to a patient a first pharmaceutical composition comprising a first drug and a GI enzyme inhibitor. Such methods are of particular use for administration to patient who is in need of therapy with the first drug of the first pharmaceutical composition, but who may be at particular risk of adverse side effects of a drug-drug interaction. Examples of such patients can include patients to whom a second drug as described herein has been previously prescribed (e.g., with direction regarding dosing, e.g., or to avoid co-administration of the first drug and a second drug), who have access to such second drugs, or who may seek access to such second drugs (e.g., patients having a history of addictive behavior).

First Pharmaceutical Compositions

In general, the GI enzyme inhibitor of the pharmaceutical composition is selected so as to provide for inhibition of GI enzyme-mediated cleavage of the second drug ingested by the patient, which second drug is a GI enzyme-cleavable prodrug ingested by a patient.

The pharmaceutical composition according to the embodiments can further comprise a pharmaceutically acceptable carrier. The composition is conveniently formulated in a form suitable for oral (including buccal and sublingual) administration, for example as a tablet, capsule, thin film, powder, suspension, solution, syrup, dispersion or emulsion. The composition can contain components conventional in pharmaceutical preparations, e.g. one or more carriers, binders, lubricants, excipients (e.g., to impart controlled release characteristics), pH modifiers, sweeteners, bulking agents, coloring agents or further active agents.

Patients can be humans, and also other mammals, such as livestock, zoo animals and companion animals, such as a cat, dog or horse.

Amounts of First Pharmaceutical Composition for Administration

The amount of first pharmaceutical composition to be administered to a patient is generally an amount sufficient to provide an effective dose of the first drug. Such effective doses can be based upon guidance for the first drug of interest.

The amount of a GI enzyme inhibitor of the first pharmaceutical composition to be administered to the patient is selected so as to be effective to decrease release of second drug administered as a GI enzyme-cleavable prodrug, thus reducing exposure of released second drug, to facilitate reduction of severity of adverse side effect(s) that can result from interaction of the first drug and second drug. The amount of GI enzyme inhibitor can be selected to, for example, to reduce GI enzyme-mediated cleavage of prodrug to a level that provides for a blood level of released second drug that is below a blood level range associated with increased risk of an adverse side effect (e.g., respiratory depression). The amount of GI enzyme inhibitor of the first pharmaceutical composition can vary according to, for example, the expected dose of the particular prodrug, the potency of the GI enzyme inhibitor of the first pharmaceutical composition, and other factors, such as the species, age, weight, sex and condition of the patient, manner of administration and judgment of the prescribing physician.

The present disclosure provides a method for treating alcohol dependence, seizures, anxiety, generalized anxiety disorder, panic, panic disorder, agitation and insomnia.

The present disclosure provides a method for treating anxiolysis, analgesia, sedation, somnolence, cognitive/memory impairment, dissociation, muscle relaxation, lowered blood pressure/heart rate, respiratory depression, anesthesia, and anticonvulsant effects.

It should be noted that patients who receive therapy using a first pharmaceutical composition of the present disclosure may also have or be at risk of a condition amenable to treatment with an opioid or with amphetamine.

For example, patients who receive therapy using a first pharmaceutical composition comprising a first drug and a GI enzyme inhibitor may include patients suffering from, or at risk of suffering from, pain. As such the patients may be receiving therapy for treatment or prevention of pain including, but not limited to include, acute pain, chronic pain, neuropathic pain, acute traumatic pain, arthritic pain, osteoarthritic pain, rheumatoid arthritic pain, muscular skeletal pain, post-dental surgical pain, dental pain, myofascial pain, cancer pain, visceral pain, diabetic pain, muscular pain, post-herpetic neuralgic pain, chronic pelvic pain, endometriosis pain, pelvic inflammatory pain and child birth related pain. Acute pain includes, but is not limited to, acute traumatic pain or post-surgical pain. Chronic pain includes, but is not limited to, neuropathic pain, arthritic pain, osteoarthritic pain, rheumatoid arthritic pain, muscular skeletal pain, dental pain, myofascial pain, cancer pain, diabetic pain, visceral pain, muscular pain, post-herpetic neuralgic pain, chronic pelvic pain, endometriosis pain, pelvic inflammatory pain and back pain.

For example, patients who receive therapy using a first pharmaceutical composition comprising a first drug and a GI enzyme inhibitor may include patients being treated for condition such as, but not limited to, Attention Deficit Hyperactivity Disorder (ADHD), Chronic Fatigue Syndrome (CFS), brain injuries, narcolepsy, obesity, etc. The present disclosure provides use of an amphetamine prodrug in the treatment of ADHD, CFS, brain injury, narcolepsy, or obesity. The present disclosure provides use of an amphetamine prodrug in the prevention of ADHD, CFS, brain injury, narcolepsy, or obesity.

Methods Used to Determine Relative Amounts of a First Drug and GI Enzyme Inhibitor in a First Pharmaceutical Composition

A first pharmaceutical composition that provide for a desired effect on release of a drug from a co-ingested GI enzyme-cleavable prodrug can be made determined by assessing relative amounts of a selected GI enzyme inhibitor effective to provide inhibition of release of drug following ingestion by a patient. Assays can be conducted in vitro, in vivo and/or ex vivo.

For example, in vitro assays can be conducted by combining a first pharmaceutical composition of a first drug with or without a GI enzyme inhibitor and a selected prodrug with a GI enzyme (e.g., trypsin) in a reaction mixture. The GI enzyme can be provided in the reaction mixture in an amount sufficient to catalyze cleavage of the prodrug. Assays are conducted under suitable conditions, and optionally may be under conditions that mimic those found in a GI tract of a subject, e.g., human. “Prodrug conversion” refers to release of drug from prodrug. Prodrug conversion can be assessed by detecting a level of a product of prodrug conversion (e.g., released drug) and/or by detecting a level of prodrug that is maintained in the presence of the GI enzyme. Prodrug conversion can also be assessed by detecting the rate at which a product of prodrug conversion occurs or the rate at which prodrug disappears. An increase in released drug, or a decrease in prodrug, indicate prodrug conversion has occurred.

In vivo assays involving administration of a first pharmaceutical composition of a first drug with or without a GI enzyme inhibitor and a selected prodrug to an animal (e.g., a human or non-human animal, e.g., rat, dog, pig, etc.) also find use in assessiong the suitability of a GI enzyme inhibitor for use in a first pharmaceutical composition. Administration can be enteral (e.g., oral administration). Prodrug conversion can be detected by, for example, detecting a product of prodrug conversion (e.g., released drug or a metabolite of released drug) or detecting prodrug in blood or plasma of the animal at a desired time point(s) following administration.

Ex vivo assays, such as a gut loop or inverted gut loop assay, can assess activity of a GI enzyme inhibitor of a first pharmaceutical composition on a prodrug by, for example, administration of the prodrug and first pharmaceutical composition to a ligated section of the intestine of an animal. Prodrug conversion can be detected by, for example, detecting a product of prodrug conversion (e.g., released drug or a metabolite of released drug) or detecting prodrug in the ligated gut loop of the animal at a desired time point(s) following administration.

Inhibitors for first pharmaceutical compositions of the present disclosure are generally selected based on, for example, activity in interacting with the GI enzyme(s) that mediate release of drug from a prodrug with which the first pharmaceutical composition may be co-dosed. Such assays can be conducted in the presence of enzyme either with or without prodrug. Inhibitors can also be selected according to properties such as half-life in the GI system, potency, avidity, affinity, molecular size and/or enzyme inhibition profile (e.g., steepness of inhibition curve in an enzyme activity assay, inhibition initiation rate). In addition, inhibitors can also be selected so as to avoid reduction of activity of the first drug with which the GI enzyme inhibitor is to be co-formulation in a first pharmaceutical composition.

One example of a method for identifying a GI enzyme inhibitor suitable for formulation in a first pharmaceutical composition comprises combining a prodrug (e.g., a phenol-modified opioid prodrug), a GI enzyme inhibitor (e.g., a trypsin inhibitor), and a GI enzyme (e.g., trypsin) in a reaction mixture and detecting prodrug conversion. Such a combination is tested for an interaction between the prodrug, inhibitor and enzyme, i.e., tested to determine how the inhibitor will interact with the enzyme that mediates enzymatically-controlled release of the drug from the prodrug. In one embodiment, a decrease in prodrug conversion in the presence of the GI enzyme inhibitor as compared to prodrug conversion in the absence of the GI enzyme inhibitor indicates the GI enzyme inhibitor is suitable for formulation in a first pharmaceutical composition with a first drug. The first drug can also be included in the reaction mixture (either with or without the prodrug) to assess retention of activity of the first drug in the presence of the GI enzyme inhibitor. Such a method can be an in vitro assay.

In another method, suitability of a GI enzyme inhibitor for formulation in a first pharmaceutical composition is assessed by administering to an animal a prodrug (e.g., a phenol-modified opioid prodrug) and a GI enzyme inhibitor (e.g., a trypsin inhibitor) and detecting prodrug conversion. Optionally, the first drug to be formulated with the GI enzyme inhibitor can be administered (e.g., either with or without the prodrug) to assess retention of activity of the first drug in the presence of the GI enzyme inhibitor. In one embodiment, a decrease in prodrug conversion in the presence of the GI enzyme inhibitor as compared to prodrug conversion in the absence of the GI enzyme inhibitor indicates the GI enzyme inhibitor is suitable for formulation in a first pharmaceutical composition. Such a method can be an in vivo assay; for example, the prodrug, GI enzyme inhibitor, and/or first drug can be administered orally. Such a method can also be an ex vivo assay; for example, the prodrug, GI enzyme inhibitor, and/or first drug can be administered orally or to a tissue, such as an intestine, that is at least temporarily exposed. Detection can occur in the blood or plasma or respective tissue. As used herein, tissue refers to the tissue itself and can also refer to contents within the tissue.

One embodiment is a method for identifying a GI enzyme inhibitor suitable for formulation in a first pharmaceutical composition wherein the method comprises administering a prodrug and a gastrointestinal (GI) enzyme inhibitor to an animal tissue that has removed from an animal and detecting prodrug conversion. The first drug can also be administered (either with or without the prodrug) to assess retention of activity of the first drug in the presence of the GI enzyme inhibitor. In one embodiment, a decrease in prodrug conversion in the presence of the GI enzyme inhibitor as compared to prodrug conversion in the absence of the GI enzyme inhibitor indicates the GI enzyme inhibitor is suitable for formulation in a first pharmaceutical composition.

In vitro assays can be conducted by combining a prodrug, an inhibitor and a GI enzyme in a reaction mixture. The GI enzyme can be provided in the reaction mixture in an amount sufficient to catalyze cleavage of the prodrug, and assays conducted under suitable conditions, optionally under conditions that mimic those found in a GI tract of a subject, e.g., human. The first drug can also be included in the reaction mixture (either with or without the prodrug) to assess retention of activity of the first drug in the presence of the GI enzyme inhibitor. Prodrug conversion can be assessed by detecting a level of a product of prodrug conversion (e.g., released drug) and/or by detecting a level of prodrug maintained in the presence of the GI enzyme. Prodrug conversion can also be assessed by detecting the rate at which a product of prodrug conversion occurs or the rate at which prodrug disappears. Prodrug conversion that is modified in the presence of inhibitor as compared to a level of prodrug conversion in the absence of inhibitor indicates the inhibitor is suitable for attenuation of prodrug conversion and for use in a dose unit. Reaction mixtures having a fixed amount of prodrug and increasing amounts of inhibitor, or a fixed amount of inhibitor and increasing amounts of prodrug, can be used to identify relative amounts of prodrug and inhibitor which provide for a desired modification of prodrug conversion. Such amounts of inhibitor can then be provided in a first pharmaceutical composition with the first drug of interest.

In vivo assays can assess combinations of prodrugs and inhibitors by co-dosing of prodrug and inhibitor to an animal. Such co-dosing can be enteral. “Co-dosing” refers to administration of prodrug and inhibitor as separate doses or a combined dose (i.e., in the same formulation). The first drug can also be administered (either with or without the prodrug) to assess retention of activity of the first drug in the presence of the GI enzyme inhibitor. Prodrug conversion can be detected by, for example, detecting a product of prodrug conversion (e.g., released drug or drug metabolite) or detecting prodrug in blood or plasma of the animal at a desired time point(s) following administration.

Use of the Methods to Reduce Risk of Adverse Side Effects in Connection with Patients Treated with Dose Units Comprising a Combination of Prodrug and Inhibitor Having a Desired Pharmacokinetic Profile

The first pharmaceutical compositions of the present disclosure can be used in connection with reducing risk of adverse side effects due to interaction with a second drug co-ingested as a GI-enzyme cleavable prodrug, where the GI-enzyme cleavable prodrug was administered as a dose unit comprising the prodrug and a GI enzyme inhibitor. Such dose units of prodrug and inhibitor can provide for a desired pharmacokinetic (PK) profile. Dose units of prodrug and inhibitor can provide a modified PK profile compared to a reference PK profile as disclosed herein. It will be appreciated that a modified PK profile can provide for a modified pharmacodynamic (PD) profile. Ingestion of multiples of such a dose unit can also provide a desired PK profile.

Unless specifically stated otherwise, “dose unit” as used herein refers to a combination of a GI enzyme-cleavable prodrug (e.g., trypsin-cleavable prodrug) and a GI enzyme inhibitor (e.g., a trypsin inhibitor). A “single dose unit” is a single unit of a combination of a GI enzyme-cleavable prodrug (e.g., trypsin-cleavable prodrug) and a GI enzyme inhibitor (e.g., trypsin inhibitor), where the single dose unit provide a therapeutically effective amount of drug (i.e., a sufficient amount of drug to effect a therapeutic effect, e.g., a dose within the respective drug's therapeutic window, or therapeutic range). “Multiple dose units” or “multiples of a dose unit” or a “multiple of a dose unit” refers to at least two single dose units.

As used herein, a “PK profile” refers to a profile of drug concentration in blood or plasma. Such a profile can be a relationship of drug concentration over time (i.e., a “concentration-time PK profile”) or a relationship of drug concentration versus number of doses ingested (i.e., a “concentration-dose PK profile”.) A PK profile is characterized by PK parameters. As used herein, a “PK parameter” refers to a measure of drug concentration in blood or plasma, such as: 1) “drug Cmax”, the maximum concentration of drug achieved in blood or plasma; 2) “drug Tmax”, the time elapsed following ingestion to achieve Cmax; and 3) “drug exposure”, the total concentration of drug present in blood or plasma over a selected period of time, which can be measured using the area under the curve (AUC) of a time course of drug release over a selected period of time (t). Modification of one or more PK parameters provides for a modified PK profile.

For purposes of describing the features of dose units of the present disclosure, “PK parameter values” that define a PK profile include drug Cmax (e.g., phenolic opioid Cmax), total drug exposure (e.g., area under the curve) (e.g., phenolic opioid exposure) and 1/(drug Tmax) (such that a decreased 1/Tmax is indicative of a delay in Tmax relative to a reference Tmax) (e.g., 1/phenolic opioid Tmax). Thus a decrease in a PK parameter value relative to a reference PK parameter value can indicate, for example, a decrease in drug Cmax, a decrease in drug exposure, and/or a delayed Tmax.

Dose units of prodrug and inhibitor can be adapted to provide for a modified PK profile, e.g., a PK profile that is different from that achieved from dosing a given dose of prodrug in the absence of inhibitor (i.e., without inhibitor). For example, dose units can provide for at least one of decreased drug Cmax, delayed drug Tmax and/or decreased drug exposure compared to ingestion of a dose of prodrug in the same amount but in the absence of inhibitor. Such a modification is due to the inclusion of an inhibitor in the dose unit.

As used herein, “a pharmacodynamic (PD) profile” refers to a profile of the efficacy of a drug in a patient (or subject or user), which is characterized by PD parameters. “PD parameters” include “drug Emax” (the maximum drug efficacy), “drug EC50” (the concentration of drug at 50% of the Emax), and side effects.

A dose unit of prodrug and inhibitor can be adapted to provide for a desired PK profile (e.g., a concentration-time PK profile) following ingestion of a single dose. A dose unit can be adapted to provide for a desired PK profile (e.g., a concentration-dose PK profile) following ingestion of multiple dose units (e.g., at least 2, at least 3, at least 4 or more dose units).

Dose Units of Prodrug and Inhibitor Providing Modified PK Profiles

A combination of a prodrug and an inhibitor in a dose unit can provide a desired (or “pre-selected”) PK profile (e.g., a concentration-time PK profile) following ingestion of a single dose. The PK profile of such a dose unit can be characterized by one or more of a pre-selected drug Cmax, a pre-selected drug Tmax or a pre-selected drug exposure. The PK profile of the dose unit can be modified compared to a PK profile achieved from the equivalent dosage of prodrug in the absence of inhibitor (i.e., a dose that is the same as the dose unit except that it lacks inhibitor).

A modified PK profile can have a decreased PK parameter value relative to a reference PK parameter value (e.g., a PK parameter value of a PK profile following ingestion of a dosage of prodrug that is equivalent to a dose unit except without inhibitor). For example, a dose unit can provide for a decreased drug Cmax, decreased drug exposure, and/or delayed drug Tmax.

Dose units that provide for a modified PK profile (e.g., a decreased drug Cmax and/or delayed drug Tmax as compared to, a PK profile of drug or a PK profile of prodrug without inhibitor), find use in tailoring of drug dose according to a patient's needs (e.g., through selection of a particular dose unit and/or selection of a dosage regimen), reduction of side effects, and/or improvement in patient compliance (as compared to side effects or patient compliance associated with drug or with prodrug without inhibitor). As used herein, “patient compliance” refers to whether a patient follows the direction of a clinician (e.g., a physician) including ingestion of a dose that is neither significantly above nor significantly below that prescribed. Such dose units also reduce the risk of misuse, abuse or overdose by a patient as compared to such risk(s) associated with drug or prodrug without inhibitor. For example, dose units with a decreased drug Cmax provide less reward for ingestion than does a dose of the same amount of drug, and/or the same amount of prodrug without inhibitor.

Dose Units Providing Modified PK Profiles Upon Ingestion of Multiple Dose Units

A dose unit of prodrug and inhibitor can be adapted to provide for a desired PK profile (e.g., a concentration-time PK profile or concentration-dose PK profile) following ingestion of multiples of a dose unit (e.g., at least 2, at least 3, at least 4, or more dose units). A concentration-dose PK profile refers to the relationship between a selected PK parameter and a number of single dose units ingested. Such a profile can be dose proportional, linear (a linear PK profile) or nonlinear (a nonlinear PK profile). A modified concentration-dose PK profile can be provided by adjusting the relative amounts of prodrug and inhibitor contained in a single dose unit and/or by using a different prodrug and/or inhibitor.

Dose units that provide for concentration-dose PK profiles when multiples of a dose unit are ingested find use in tailoring of a dosage regimen to provide a therapeutic level of released drug while reducing the risk of overdose, misuse, or abuse. Such reduction in risk can be compared to a reference, e.g., to administration of drug alone or prodrug alone. In one embodiment, risk is reduced compared to administration of a drug or prodrug that provides a proportional concentration-dose PK profile. A dose unit that provides for a concentration-dose PK profile can reduce the risk of patient overdose through inadvertent ingestion of dose units above a prescribed dosage. Such a dose unit can reduce the risk of patient misuse (e.g., through self-medication). Such a dose unit can discourage abuse through deliberate ingestion of multiple dose units. For example, a dose unit that provides for a biphasic concentration-dose PK profile can allow for an increase in drug release for a limited number of dose units ingested, after which an increase in drug release with ingestion of more dose units is not realized. In another example, a dose unit that provides for a concentration-dose PK profile of zero slope can allow for retention of a similar drug release profile regardless of the number of dose units ingested.

Ingestion of multiples of a dose unit can provide for adjustment of a PK parameter value relative to that of ingestion of multiples of the same dose (either as drug alone or as a prodrug) in the absence of inhibitor such that, for example, ingestion of a selected number (e.g., 2, 3, 4 or more) of a single dose unit provides for a decrease in a PK parameter value compared to ingestion of the same number of doses in the absence of inhibitor.

Combinations of relative amounts of prodrug and inhibitor that provide for a desired PK profile can be identified by dosing animals with a fixed amount of prodrug and increasing amounts of inhibitor, or with a fixed amount of inhibitor and increasing amounts of prodrug. One or more PK parameters can then be assessed, e.g., drug Cmax, drug Tmax, and drug exposure. Relative amounts of prodrug and inhibitor that provide for a desired PK profile are identified as amounts of prodrug and inhibitor for use in a dose unit. The PK profile of the prodrug and inhibitor combination can be, for example, characterized by a decreased PK parameter value relative to prodrug without inhibitor. A decrease in the PK parameter value of an inhibitor-to-prodrug combination (e.g., a decrease in drug Cmax, a decrease in 1/drug Tmax (i.e., a delay in drug Tmax) or a decrease in drug exposure) relative to a corresponding PK parameter value following administration of prodrug without inhibitor can be indicative of an inhibitor-to-prodrug combination that can provide a desired PK profile. Assays can be conducted with different relative amounts of inhibitor and prodrug.

In vivo assays can be used to identify combinations of prodrug and inhibitor that provide for dose units that provide for a desired concentration-dose PK profile following ingestion of multiples of the dose unit (e.g., at least 2, at least 3, at least 4 or more). Ex vivo assays can be conducted by direct administration of prodrug and inhibitor into a tissue and/or its contents of an animal, such as the intestine, including by introduction by injection into the lumen of a ligated intestine (e.g., a gut loop, or intestinal loop, assay, or an inverted gut assay). An ex vivo assay can also be conducted by excising a tissue and/or its contents from an animal and introducing prodrug and inhibitor into such tissues and/or contents.

For example, a dose of prodrug that is desired for a single dose unit of prodrug and inhibitor is selected (e.g., an amount that provides an efficacious plasma drug level). A multiple of single dose units for which a relationship between that multiple and a PK parameter to be tested is then selected. For example, if a concentration-dose PK profile is to be designed for ingestion of 2, 3, 4, 5, 6, 7, 8, 9 or 10 dose units, then the amount of prodrug equivalent to ingestion of that same number of dose units is determined (referred to as the “high dose”). The multiple of dose units can be selected based on the number of ingested pills at which drug Cmax is modified relative to ingestion of the single dose unit. If, for example, the profile is to provide for abuse deterrence, then a multiple of 10 can be selected, for example. A variety of different inhibitors (e.g., from a panel of inhibitors) can be tested using different relative amounts of inhibitor and prodrug. Assays can be used to identify suitable combination(s) of inhibitor and prodrug to obtain a single dose unit that is therapeutically effective, wherein such a combination, when ingested as a multiple of dose units, provides a modified PK parameter compared to ingestion of the same multiple of drug or prodrug alone (wherein a single dose of either drug or prodrug alone releases into blood or plasma the same amount of drug as is released by a single dose unit).

Increasing amounts of inhibitor are then co-dosed to animals with the high dose of prodrug. The dose level of inhibitor that provides a desired drug Cmax following ingestion of the high dose of prodrug is identified and the resultant inhibitor-to-prodrug ratio determined.

Prodrug and inhibitor are then co-dosed in amounts equivalent to the inhibitor-to-prodrug ratio that provided the desired result at the high dose of prodrug. The PK parameter value of interest (e.g., drug Cmax) is then assessed. If a desired PK parameter value results following ingestion of the single dose unit equivalent, then single dose units that provide for a desired concentration-dose PK profile are identified. For example, where a zero dose linear profile is desired, the drug Cmax following ingestion of a single dose unit does not increase significantly following ingestion of a multiple number of the single dose units.

### Methods for Manufacturing, Formulating, and Packaging Pharmaceutical Compositions

Pharmaceutical composition of the present disclosure can be made using manufacturing methods available in the art and can be of a variety of forms suitable for enteral (including oral, buccal and sublingual) administration, for example as a tablet, capsule, thin film, powder, suspension, solution, syrup, dispersion or emulsion. The pharmaceutical compositions can contain components conventional in pharmaceutical preparations, e.g. one or more carriers, binders, lubricants, excipients (e.g., to impart controlled release characteristics), pH modifiers, flavoring agents (e.g., sweeteners), bulking agents, coloring agents or further active agents. Pharmaceutical compositions of the present disclosure can include can include an enteric coating or other component(s) to facilitate protection from stomach acid, where desired.

Pharmaceutical compositions can be provided in dosage forms of any suitable size or shape. The dosage form can be of any shape suitable for enteral administration, e.g., ellipsoid, lenticular, circular, rectangular, cylindrical, and the like.

Pharmaceutical compositions can be provided in dry form of, for example, a total weight of from about 1 microgram to about 1 gram, and can be from about 5 micrograms to 1.5 grams, from about 50 micrograms to 1 gram, from about 100 micrograms to 1 gram, from 50 micrograms to 750 milligrams, and may be from about 1 microgram to 2 grams.

Pharmaceutical compositions can comprise components in any relative amounts. For example, pharmaceutical compositions can be from about 0.1% to 99% by weight of active ingredients (e.g., first drug and inhibitor; e.g., prodrug and inhibitor) per total weight of the dosage form (0.1% to 99% total combined weight of first drug and inhibitor per total weight of single dosage form). In some embodiments, dosage forms can be from 10% to 50%, from 20% to 40%, or about 30% by weight of active ingredients per total weight.

Dosage forms can be provided in a variety of different forms and optionally provided in a manner suitable for storage. For example, dosage forms can be disposed within a container suitable for containing a pharmaceutical composition. The container can be, for example, a bottle (e.g., with a closure device, such as a cap), a blister pack (e.g., which can provide for enclosure of one or more pills per blister), a vial, flexible packaging (e.g., sealed Mylar or plastic bags), an ampule (for single or multiple doses in solution), a dropper, thin film, a tube and the like.

Containers can include a cap (e.g., screw cap) that is removably connected to the container over an opening through which the dosage forms disposed within the container can be accessed.

Containers can include a seal which can serve as a tamper-evident and/or tamper-resistant element, which seal is disrupted upon access. Such seal elements can be, for example, a frangible element that is broken or otherwise modified upon access to the container. Examples of such frangible seal elements include a seal positioned over a container opening such that access to the interior of the container requires disruption of the seal (e.g., by peeling and/or piercing the seal). Examples of frangible seal elements include a frangible ring disposed around a container opening and in connection with a cap such that the ring is broken upon opening of the cap.

Dry and liquid dosage forms can be placed in a container (e.g., bottle or package, e.g., a flexible bag) of a size and configuration adapted to maintain stability of the pharmaceutical composition over a desired period. The containers can be sealed or resealable. The containers can packaged in a carton (e.g., for shipment from a manufacturer to a pharmacy or other dispensary). Such cartons can be boxes, tubes, or of other configuration, and may be made of any material (e.g., cardboard, plastic, and the like). The packaging system and/or containers disposed therein can have one or more affixed labels (e.g., to provide information such as lot number, manufacturer, and the like).

The container can include a moisture barrier and/or light barrier, e.g., to facilitate maintenance of stability of the active ingredients contained therein. Where the pharmaceutical compositions is in a dry dosage form, the container can include a desiccant pack which is disposed within the container. The container can be adapted to contain a single or multiples of doses of a pharmaceutical composition. The container can include a dispensing control mechanism, such as a lock out mechanism that facilitates maintenance of dosing regimen, e.g., dosing regimen of a prodrug pharmaceutical composition and a dosing regimen of a pharmaceutical composition comprising a first drug and a GI enzyme inhibitor.

Pharmaceutical compositions can be provided in solid or semi-solid form, and can be a dry. “Dry” refers to a pharmaceutical composition is in other than in a completely liquid form, e.g., tablets, capsules (e.g., solid capsules, capsules containing liquid), thin film, microparticles, granules, powder and the like. Pharmaceutical compositions can be provided as a liquid, where the doses can be provided as single or multiple doses of a formulation. Single doses of a dry or liquid dosage form can be disposed within a sealed container, and sealed containers optionally provided in a packaging system, e.g., to provide for a prescribed number of doses, to provide for shipment of dose units, and the like.

Pharmaceutical compositions can be formulated such that the first drug and inhibitor are present in the same carrier, e.g., solubilized or suspended within the same matrix. Alternatively, pharmaceutical compositions can be composed of two or more portions, where the first drug and inhibitor can be provided in the same or different portions, and can be provided in adjacent or non-adjacent portions.

Pharmaceutical compositions can be provided in a container in which they are disposed, and may be provided as part of a packaging system (optionally with instructions for use). For example, pharmaceutical compositions containing a first drug and GI enzyme inhibitor can be provided in separate containers, which containers can be disposed with in a larger container (e.g., to facilitate protection of dose units for shipment). For example, one or more pharmaceutical compositions as described herein can be provided in separate containers, where pharmaceutical compositions of different composition are provided in separate containers, and the separate containers disposed within package for dispensing.

In another example, pharmaceutical compositions can be provided in a double-chambered dispenser where a first chamber contains a first drug formulation and a second chamber contains an inhibitor formulation. For example, the dispenser can be adapted to provide for mixing of a first drug formulation and an inhibitor formulation prior to ingestion. For example, the two chambers of the dispenser can be separated by a removable wall (e.g., frangible wall) that is broken or removed prior to administration to allow mixing of the formulations of the two chambers. The first and second chambers can terminate into a dispensing outlet, optionally through a common chamber. The formulations can be provided in dry or liquid form, or a combination thereof. For example, the formulation in the first chamber can be liquid and the formulation in the second chamber can be dry, both can be dry, or both can be liquid.

Pharmaceutical compositions that provide for controlled release of first drug, of inhibitor, or of both first drug and inhibitor are contemplated by the present disclosure, where “controlled release” refers to release of one or both of first drug and inhibitor from the composition over a selected period of time and/or in a pre-selected manner.

In addition, the present disclosure provides containers which provide a first pharmaceutical composition comprising a first drug and a GI enzyme inhibitor and a second pharmaceutical composition comprising a GI enzyme cleavable prodrug (optionally formulated with a GI enzyme inhibitor to provide for a desired PK profile, as described herein), where the GI enzyme inhibitor in the first composition is effective to attenuate release of second drug from the prodrug. Such containers may be provided in the form of packaging which provides the prodrug and the first pharmaceutical composition in separate compartments. The packaging can thus implicitly and/or explicitly include direction for ingestion of the prodrug and the first pharmaceutical composition so as to provide for therapeutic benefit of a second drug administered as the prodrug (e.g., an opioid or amphetamine, or derivative thereof) and therapeutic benefit of the first drug, while reducing the risk of adverse side effects of drug-drug interaction. In some embodiments, the GI enzyme cleavable prodrug can be provided as a second pharmaceutical composition comprising the prodrug and a GI enzyme inhibitor which provides for a modulation of release of second drug from the prodrug, e.g., to provide a desired PK profile of second drug as described above.

### EXAMPLES

The following examples are put forth so as to provide those of ordinary skill in the art with a complete disclosure and description of how to make and use the embodiments, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used.

### Synthesis of Phenolic Opioid Prodrugs

### Example 1

### Synthesis of [2-((S)-2-malonylamino-4-amino-pentanoyl amino)-ethyl]-ethyl-carbamic acid hydromorphone ester (Compound PC-5)

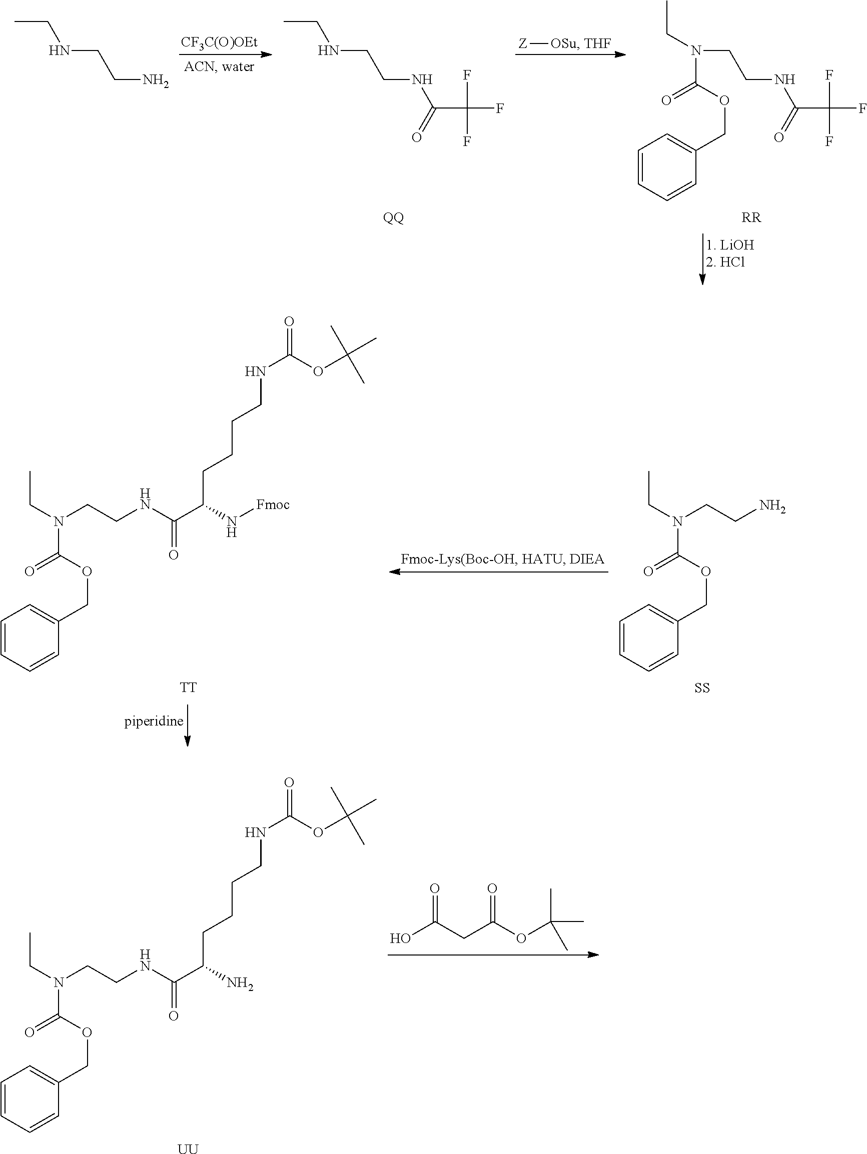

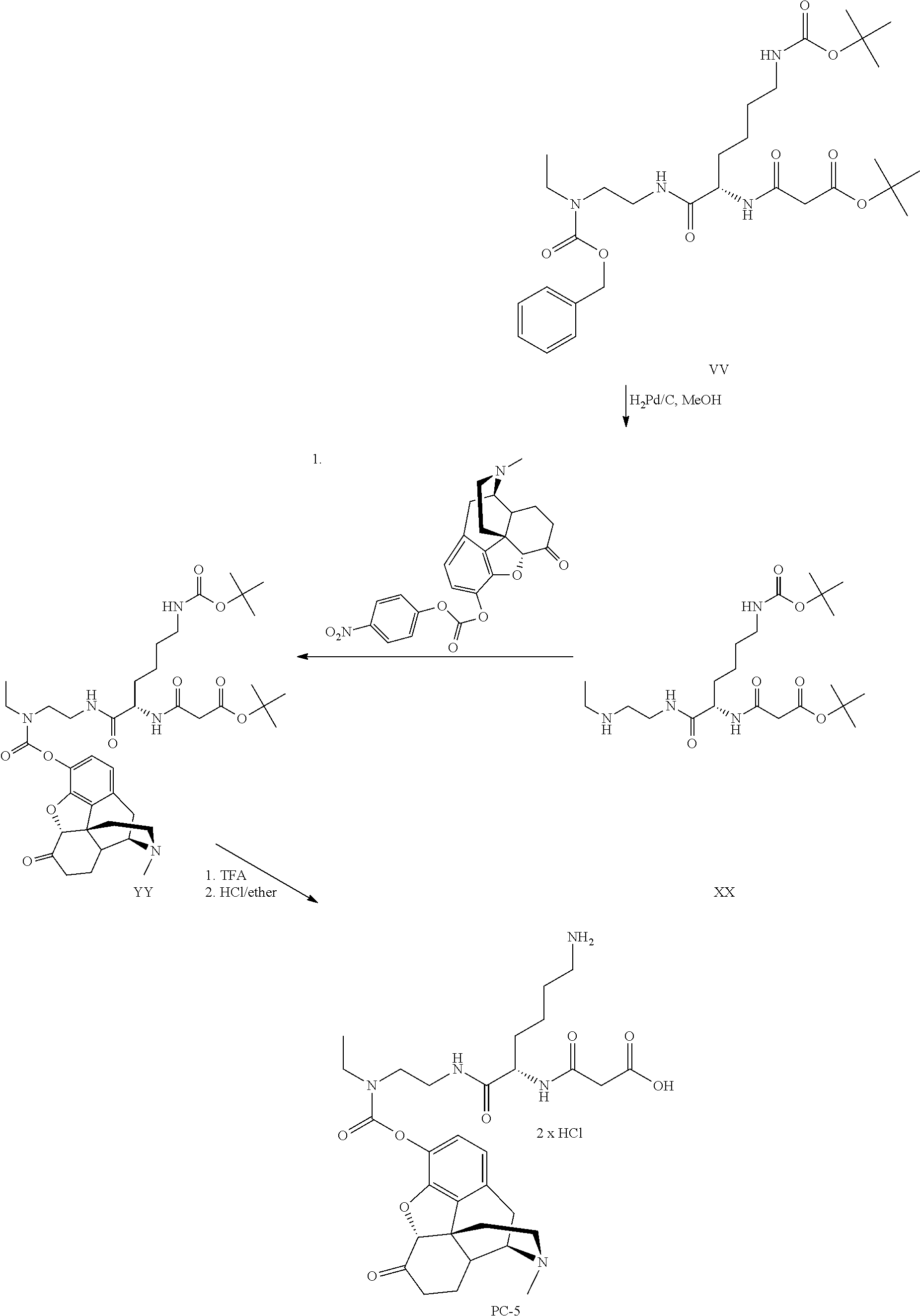

### Synthesis of 2,2,2-trifluoro-N-(2-ethylamino-ethyl)-acetamide (QQ)

A solution of N-ethylethylenediamine (10.0 g, 113.4 mmol) and ethyl trifluoroacetate (32.0 mL, 261 mmol) in a mixture of acetonitrile (110 mL) and water (2.5 mL, 139 mmol) was refluxed with stirring overnight (˜18 hours (hr, h)). Solvents were evaporated in vacuo. Residue was re-evaporated with isopropanol (3×100 mL). Residue was dissolved in dichloromethane (500 mL) and left overnight at room temperature (rt). The formed crystals were filtered, washed with dichloromethane (100 mL) and dried in vacuo to provide compound QQ (24.6 g, 82.4 mmol) as a white solid powder.

### Synthesis of ethyl-[2-(2,2,2-trifluoro-acetylamino)-ethyl]-carbamic acid benzyl ester (RR)

A solution of compound QQ (24.6 g, 82.4 mmol) and DIEA (14.3 mL, 82.4 mmol) in THF (100 mL) was cooled to −5° C., followed by the addition a solution of N-(benzyloxycarbonyl)succinimide (20.3 g, 81.6 mmol) in THF (75 mL) dropwise over 20 min. The temperature of the reaction mixture was raised to room temperature and stirring was continued for an additional 30 minutes (min). Solvents were evaporated and the residue was dissolved in ethyl acetate (500 mL). The organic layer was extracted with 5% aq. NaHCO₃ (2×100 mL) and brine (100 mL). The organic layer was evaporated to provide compound RR (24.9 g, 78.2 mmol) as a yellowish oil. LC-MS [M+H] 319.0 (C₁₄H₁₇F₃N₂O₃+H, calc: 319.2). Compound RR was used without further purification.

### Synthesis of (2-Amino-ethyl)-ethyl-carbamic acid benzyl ester (SS)

To a solution of compound RR (24.9 g, 78.2 mmol) in methanol (300 mL) was added a solution of LiOH (3.8 g, 156 mmol) in water (30 mL). The reaction mixture was stirred at room temperature for 5 h. Next the solvents were evaporated to 75% of initial volume followed by dilution with water (200 mL). The solution was extracted with ethyl acetate (200 mL×2) and the organic layer was washed with brine (100 mL), dried over MgSO₄ and evaporated in vacuo. Residue was dissolved in ether (200 mL) and treated with 2 N HCl/ether (200 mL). The formed precipitate was filtered, washed with ether and dried in vacuo to provide the hydrochloric salt of compound SS (12.1 g, 46.7 mmol) as a white solid. LC-MS [M+H] 222.9 (C₁₂H₁₈N₂O₂+H, calc: 223.2). Purity>95% (UV/254 nm).

### Synthesis of {2-[Fmoc-Lys (Boc)]-aminoethyl}-ethyl-carbamic acid benzyl ester (TT)

To a solution of Fmoc-Lys(Boc)-OH (25.02 g, 53.4 mmol, 1 eq), compound SS (13.82 g, 53.4 mmol, 1 eq) and HATU (22.3 g, 58.7 mmol, 1.1 eq) in DMF (300 mL) was added a solution of DIEA (28 mL, 160.2 mmol, 3.0 eq), cooled with an ice/water bath and stirring for 30 min. The reaction mixture was stirred at ambient temperature for 2 h. Upon completion, the reaction mixture was diluted with EtOAc (1 L) and extracted with water (2×2.5 L) and brine (500 mL). The organic layer was dried over anhydrous Na₂SO₄, filtered and then evaporated to give an oily residue, which was dried overnight in vacuo (120 mbar) to give compound TT (39.5 g) as a yellow-brown viscous solid. LC-MS [M+H] 672.5 (C₃₈H₄₈N₄O₇+H, calc: 672.7). Purity>95% (UV/254 nm). Compound TT was used without purification.

### Synthesis of {2[H-Lys(Boc)]-aminoethyl}-ethyl-carbamic acid benzyl ester (UU)

Compound TT (18.5 g, 25 mmol, 1 eq) and piperidine (3.1 mL, 31 mmol, 1.2 eq) was dissolved in ethyl acetate (125 mL), using sonication and stirring to assist in dissolving all components. The reaction mixture was stirred at ambient temperature for 5 h, monitoring the reaction progress by LC/MS. Upon completion, the solvent was then removed in vacuo to ˜15 mL, then the product was triturated with hexane (250 mL) to give an oily residue. Hexane was decanted and the residue was washed further with hexane (100 mL). The product was dried overnight in vacuo to provide compound UU (13.5 g) as a yellowish solid. LC-MS [M+H] 451.3 (C₂₃H₄₃₈N₄O₅+H, calc: 451.3). Purity>95% (UV/254 nm). Compound UU was used without purification.

### Synthesis of {2-[t-Boc-malonyl-Lys(Boc)]-aminoethyl}-ethyl-carbamic acid benzyl ester (VV)

Compound UU (12.5 g, 25.0 mmol, 1 eq), DIEA (10.9 mL, 27.5 mmol, 2.5 eq) and BOP (12.2 g, 27.5 mmol, 1.1 eq) were dissolved in DMF (20 mL), and a solution of mono-t-butyl-malonate (4.5 g, 27.5 mmol, 1.1 eq) in DMF (20 mL) was added to the reaction mixture with cooling with an ice/water bath and stirring over 30 min. The reaction was complete in 2 h, and the solvent was removed in vacuo. The residue was dissolved in ethyl acetate (700 mL) and washed with water (1.2 L) and then brine (500 mL). The organic layer was separated, and the aqueous phase was reextracted with ethyl acetate (400 mL). The combined organic phase was dried over anhydrous Na₂SO₄, and solvent was evaporated in vacuo to give an oily residue. The product was dried overnight in vacuo to give compound VV (19.2 g) as a pale yellow oil. LC-MS [M+H] 593.7 (C₃₀H₄₈N₄O₈+H, calc: 593.4). Compound VV was used without purification. Purity>95% (UV/254 nm).

### Synthesis of N-[t-Boc-malonyl-Lys(Boc)]N′-ethyl-ethane-1,2-diamine (XX)

Compound VV (19.2 g, 25 mmol) was suspended in methanol (500 mL) and filtered off from inorganic salts. A Pd/C (5% wt, 2.4 g) suspension in water (10 mL) was added, and the reaction mixture was hydrogenated (Parr apparatus, 80 psi) at ambient temperature for 2 h. Upon reaction completion, the catalyst was filtered through a pad of Celite® on sintered glass frit and washed with methanol (2×50 mL). The filtrate was evaporated in vacuo to give an oily residue. The product was dried overnight in vacuo to give compound XX (17.3 g) as a pale yellow oil. LC-MS [M+H] 459.4 (C₂₂H₄₂N₄O₆+H, calc: 459.3). Compound XX was used without purification. Purity>95% (UV/254 nm).

### Synthesis of [t-Boc-malonyl-Lys(Boc)]-ethyl-carbamic acid hydromorphone ester (YY)

A suspension of hydromorphone hydrochloride (10.5 g, 32.5 mmol, 1.3 eq) and DIPEA (5.7 mL, 32.5 mmol) in chloroform (70 mL) was sonicated in an ultrasonic bath at ambient temperature for 1 h, followed by addition of 4-nitrophenyl chloroformate (5.05 g, 25 mmol, 1 eq). The reaction mixture was sonicated in an ultrasonic bath at ambient temperature for additional 1 h, followed by the addition of a solution of compound XX (17.3 g, 25 mmol, 1 eq) and 1-hydroxybenzotriazole (5.06 g, 37.5 mmol, 1.5 eq) in DMF (50 mL). The reaction mixture was stirred overnight (˜18 h) at ambient temperature. Next, the reaction mixture was filtered through a glass frit and the solvents were evaporated in vacuo. The crude reaction mixture was dissolved in methanol (50 mL) and precipitated with ether (500 mL) to give an oily yellow residue. It was re-precipitated from methanol/ether (50 mL/500 mL) to form a viscous product, which was dried in vacuo overnight to provide crude compound YY (18.8 g, 98% yield) as a foaming pale yellow solid. LC-MS [M+H-Boc] 670.1 (C₄₀H₅₉N₅O₁₀+H-boc, calc: 670.2). Purity ˜50% (UV/254 nm).

Crude product YY (5.2 g, 5.54 mmol) was dissolved in a mixture DMSO/AcOH (10 mL/40 mL) and diluted with water (50 mL). The solution was subjected to HPLC purification: Nanosyn-Pack Microsorb (100-10) C-18 column (50×300 mm); flow rate: 100 mL/min; injection volume 50 mL; mobile phase A: 100% water, 0.1% TFA; mobile phase B: 100% ACN, 0.1% TFA; isocratic elution at 10% B in 4 min, gradient elution from 10% to 28% B in 27 min, isocratic elution at 28% B in 30 min, gradient elution from 28% B to 42% B in 29 min; detection at 254 nm. Fractions containing the desired compound were combined and concentrated in vacuo. The residue was dissolved in isopropanol (100 mL) and co-evaporated in vacuo (procedure repeated twice). The resulting solid was dried in vacuo overnight to provide compound YY (10.2 g, 48% yield) as a foaming white solid. LC-MS [M+H-Boc] 670.1 (C₄₀H₅₉N₅O₁₀+H-boc, calc: 670.2). Purity>95% (UV/254 nm).

### Synthesis of [2-((S)-2-malonylamino-4-amino-pentanoyl amino)-ethyl]-ethyl-carbamic acid hydromorphone ester (Compound PC-5)

Compound YY (10.2 g, 11.5 mmol) was dissolved in DCM (20 mL) and treated with TFA (50 mL). The reaction mixture was stirred at ambient temperature for 1 h, monitoring the reaction progress by LC/MS. Upon reaction completion, the solvent was evaporated in vacuo to afford a pale yellow oil. It was dissolved in isopropanol (20 mL) and treated with 2 N HCl/ether (100 mL, 200 mmol) to give immediately a thick white precipitate. It was diluted with ether (500 mL) and filtered off. The solid was washed with ether (2×50 mL) and hexane (2×50 mL). The solid was dried in vacuo to yield Compound PC-5: (6.8 g, 86.1% yield, 96.8% purity) by 254 nm/UV) as a white solid. LC-MS [M+H] 614.2 (C₃₁H₄₃N₅O₈+H, calc: 614.3). Retention time*: 1.93 min*−[Chromolith SpeedRod RP-18e C18 column (4.6×50 mm); flow rate 1.5 mL/min; mobile phase A: 0.1% TFA/water; mobile phase B 0.1% TFA/ACN; gradient elution from 5% B to 100% B over 9.6 min, detection 254 nm]

### Biological Data of Phenol-modified Opioid Prodrugs

### Example 2

### Oral Administration of Compound PC-1 and SBTI Trypsin Inhibitor to Rats

Hydromorphone 3-(N-methyl-N-(2-N′-acetylarginylamino)) ethylcarbamate (which can be produced as described in PCT International Publication No. WO 2007/140272, published 6 Dec. 2007, Example 3, hereinafter referred to as Compound PC-1) and SBTI (trypsin inhibitor from Glycine max (soybean) (Catalog No. 93620, ˜10,000 units per mg, Sigma-Aldrich) were each dissolved in saline.

Saline solutions of Compound PC-1 and SBTI were dosed as indicated in Table 1 via oral gavage into jugular vein-cannulated male Sprague Dawley rats that had been fasted for 16-18 hr prior to oral dosing; 4 rats were dosed per group. When SBTI was dosed, it was administered 5 minutes (min) prior to Compound PC-1. At specified time points, blood samples were drawn, quenched into methanol, centrifuged at 14,000 rpm @ 4° C., and stored at −80° C. until analysis by high performance liquid chromatography/mass spectrometry (HPLC/MS).

Table 1 indicates the results for rats administered a constant amount of Compound PC-1 and variable amounts of SBTI. Results are reported as maximum blood concentration of hydromorphone (average±standard deviation) for each group of 4 rats.

TABLE 1
Maximum concentration (Cmax) of hydromorphone in rat blood

| Compound | SBTI | Cmax (ng/mL |
|---|---|---|
| PC-1 (mg/kg) | (mg/kg) | HM) |
| 20 | 0 | 16.5 ± 5.3 |
| 20 | 10 | 8.9 ± 1.8 |
| 20 | 100 | 6.0 ± 4.0 |
| 20 | 500 | <5 |
| 20 | 1000 | <5 |

Lower limit of quantitation was 1 nanogram per milliliter (ng/mL) for the first group and 5 ng/mL for the other groups.
   The results in Table 1 indicate that SBTI attenuates Compound PC-1's ability to release hydromorphone in a dose-dependent manner that can approach approximately 100% attenuation at higher SBTI concentrations.

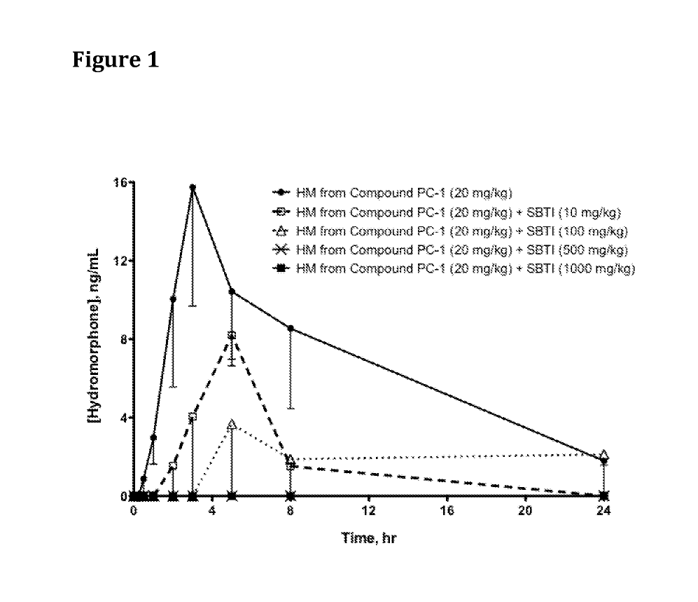

Data obtained from the rats represented in Table 1 are also provided in FIG. 1 which compares mean blood concentrations (±standard deviations) over time of hydromorphone following PO administration to rats of 20 mg/kg Compound PC-1 (a) alone (solid line with closed circle symbols), (b) with 10 mg/kg SBTI (dashed line with open square symbols), (c) with 100 mg/kg SBTI (dotted line with open triangle symbols), (d) with 500 mg/kg SBTI (solid line with X symbols) or (e) with 1000 mg/kg SBTI (solid line with closed square symbols). The results in FIG. 1 indicate that SBTI attenuation of Compound PC-1's ability to release hydromorphone suppresses Cmax and delays Tmax of such hydromorphone release into the blood of rats administered Compound PC-1 and 10, 100, 500 or 1000 mg/kg SBTI. Examples of TI attenuation of other phenolic opioid prodrugs can be found in WO 2010/045599, published 22 Apr. 2010, which is hereby incorporated by reference in its entirety.

### Example 3

### Oral Administration of Compound PC-5 Co-Dosed with Trypsin Inhibitor Compound 109 to Rats

Saline solutions of Compound PC-5 were dosed with increasing co-doses of Compound 109 (Catalog No. 3081, Tocris Bioscience, Ellisville, Mo., USA or Catalog WS38665, Waterstone Technology, Carmel, Ind., USA) as indicated in Table 13 via oral gavage into jugular vein-cannulated male Sprague Dawley rats (4 per group) that had been fasted for 16-18 hr prior to oral dosing. At specified time points, blood samples were drawn, harvested for plasma via centrifugation at 5,400 rpm at 4° C. for 5 min, and 100 microliters (μl) plasma transferred from each sample into a fresh tube containing 2 μl of 50% formic acid. The tubes were vortexed for 5-10 seconds, immediately placed in dry ice and then stored in −80° C. freezer until analysis by HPLC/MS.

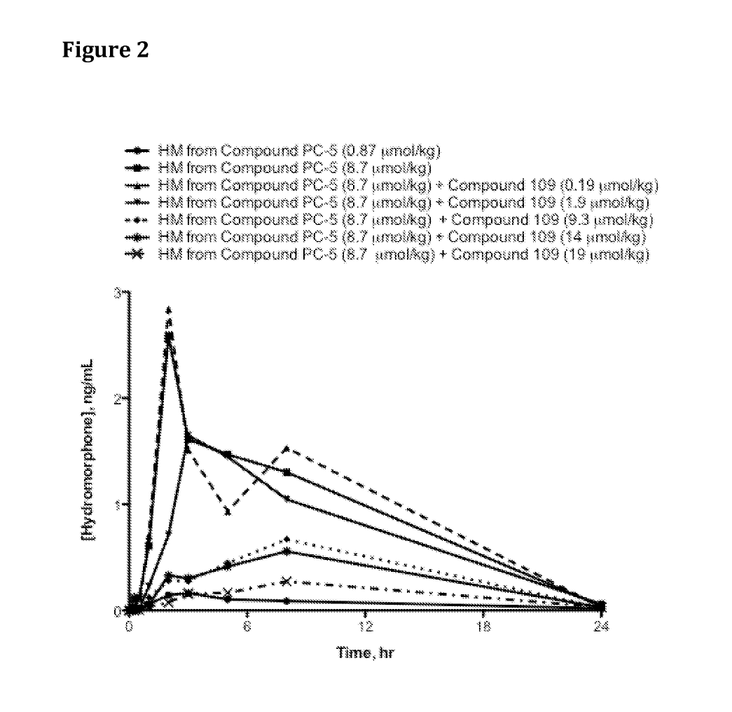

Table 2 and FIG. 2 provide hydromorphone exposure results for rats administered Compound PC-5 and increasing doses of trypsin inhibitor. Results in Table 2 are reported, for each group of 4 rats, as (a) maximum plasma concentration (Cmax) of hydromorphone (HM) (average±standard deviation), (b) time after administration of Compound PC-5 to reach maximum hydromorphone concentration (Tmax) (average±standard deviation) and (c) area under the curve (AUC) from 0 to 24 hr.

TABLE 2
Cmax, Tmax and AUC values of hydromorphone in rat plasma

| PC-5 | | Compound | Compound | | | |
|---|---|---|---|---|---|---|
| Dose, | PC-5 Dose, | 109 Dose, | 109 Dose, | HM Cmax ± sd, | | AUC ± sd, |
| mg/kg | μmol/kg | mg/kg | μmol/kg | ng/mL | Tmax ± sd, hr | ng × hr/mL |
| 0.6 | 0.87 | 0 | 0 | 0.196 ± 0.11 | 3.75 ± 2.9 | 1.33 ± 0.84 |
| 6 | 8.7 | 0 | 0 | 2.68 ± 1.2 | 2.50 ± 0.58 | 19.4 ± 5.7 |
| 6 | 8.7 | 0.1 | 0.19 | 2.84 ± 1.8 | 2.00 ± 0.0 | 19.3 ± 4.3 |
| 6 | 8.7 | 1 | 1.9 | 1.75 ± 1.0 | 3.25 ± 1.3 | 17.4 ± 8.4 |
| 6 | 8.7 | 5 | 9.3 | 0.669 ± 0.15 | 8.00 ± 0.0 | 7.54 ± 4.0 |
| 6 | 8.7 | 7.5 | 14 | 0.584 ± 0.18 | 4.56 ± 4.0 | 6.57 ± 3.5 |
| 6 | 8.7 | 10 | 19 | 0.295 ± 0.063 | 6.06 ± 3.9 | 2.29 ± 1.3 |

Lower limit of quantitation was 0.0250 ng/mL.

FIG. 2 compares mean plasma concentrations over time of hydromorphone release following PO administration of Compound PC-5 with increasing amounts of co-dosed trypsin inhibitor Compound 109.

The results in Table 2 and FIG. 2 indicate Compound 109's ability to attenuate Compound PC-5's ability to release hydromorphone in a dose dependent manner, both by suppressing Cmax and AUC and by delaying Tmax.

### Example 4

### Oral Administration of a Single Dose Unit and of Multiple Dose Units of a Composition Comprising Prodrug Compound PC-5 and Trypsin Inhibitor Compound 109 in Rats

A saline solution of a composition comprising 0.87 μmol/kg (0.6 mg/kg) Compound PC-5 and 1.9 μmol/kg (1 mg/kg) Compound 109, representative of a single dose unit, was administered via oral gavage into a group of 4 rats. It is to be noted that the mole-to-mole ratio of trypsin inhibitor-to-prodrug (109-to-PC-5) is 2.2-to-1 as such this dose unit is referred to herein as a 109-to-PC-5 (2.2-to-1) dose unit. Saline solutions representative of (a) 2 dose units (i.e., a composition comprising 1.7 μmol/kg (1.2 mg/kg) Compound PC-5 and 3.8 μmol/kg (2 mg/kg) Compound 109), (b) 3 dose units (i.e., a composition comprising 2.6 μmol/kg (1.8 mg/kg) Compound PC-5 and 5.7 μmmol/kg (3 mg/kg) Compound 109), and (c) 10 dose units (i.e., a composition comprising 8.7 μmol/kg (6 mg/kg) Compound PC-5 and 19 μmol/kg (10 mg/kg) Compound 109) of the 109-to-PC-5 (2.2-to 1) dose unit were similarly administered to additional groups of 4 rats. All rats were jugular vein-cannulated male Sprague Dawley rats that had been fasted for 16-18 hr prior to oral dosing. At specified time points, blood samples were drawn, harvested for plasma via centrifugation at 5,400 rpm at 4° C. for 5 min, and 100 microliters (μl) plasma transferred from each sample into a fresh tube containing 2 μl of 50% formic acid. The tubes were vortexed for 5-10 seconds, immediately placed in dry ice and then stored in −80° C. freezer until analysis by HPLC/MS.

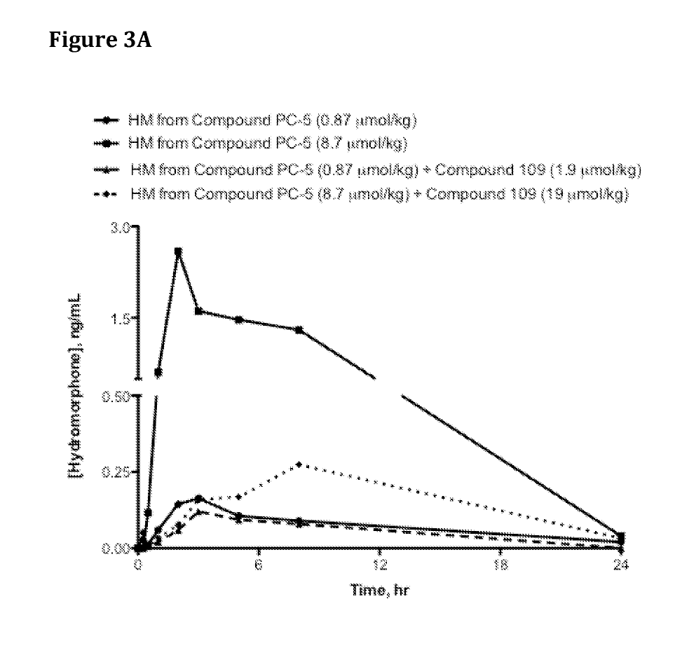

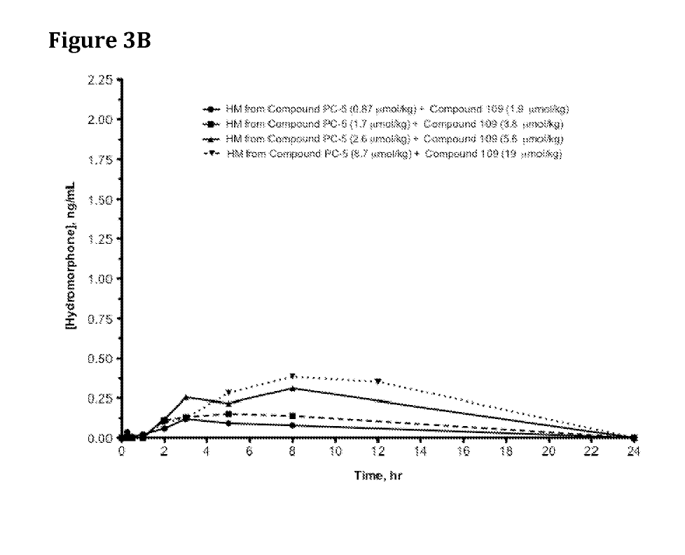

Table 3A and FIG. 3A provide hydromorphone exposure results for rats administered a single dose unit or 10 dose units of the 109-to-PC-5 (2.2-to 1) dose unit. Also provided are results for rats administered 0.87 μmol/kg (0.6 mg/kg) or 8.7 μmol/kg (6 mg/kg) of Compound PC-5 without trypsin inhibitor. Table 3B and FIG. 3B compare hydromorphone exposure results for rats administered 1, 2, 3 or 10 dose units of the 109-to-PC-5 (2.2-to 1) dose unit. Results in Table 3A and Table 3B are reported, for each group of 4 rats, as (a) maximum plasma concentration (Cmax) of hydromorphone (HM) (average±standard deviation), (b) time after administration of Compound PC-5 to reach maximum hydromorphone concentration (Tmax) (average±standard deviation) and (c) area under the curve (AUC) from 0 to 24 hr.

TABLE 3A
Cmax, Tmax and AUC values of hydromorphone in rat plasma

| PC-5 | PC-5 | Compound | Compound | | | |
|---|---|---|---|---|---|---|
| Dose, | Dose, | 109 Dose, | 109 Dose, | HM Cmax ± sd, | Tmax ± sd, | AUC ± sd, |
| mg/kg | μmol/kg | mg/kg | μmol/kg | ng/mL | hr | ng × hr/mL |
| 0.6 | 0.87 | 1 | 1.9 | 0.131 ± 0.027 | 4.25 ± 2.5 | 0.596 ± 0.24 |
| 6 | 8.7 | 10 | 19 | 0.295 ± 0.063 | 6.06 ± 3.9 | 2.29 ± 1.3 |
| 0.6 | 0.87 | 0 | 0 | 0.196 ± 0.11 | 3.75 ± 2.9 | 1.33 ± 0.84 |
| 6 | 8.7 | 0 | 0 | 2.68 ± 1.2 | 2.50 ± 0.58 | 19.4 ± 5.7 |

Lower limit of quantitation was 0.0500 ng/mL for both groups.

TABLE 3B
Cmax, Tmax and AUC values of hydromorphone in rat plasma

| PC-5 | PC-5 | Compound | Compound | | | |
|---|---|---|---|---|---|---|
| Dose, | Dose, | 109 Dose, | 109 Dose, | HM Cmax ± sd, | Tmax ± sd, | AUC ± sd, |
| mg/kg | μmol/kg | mg/kg | μmol/kg | ng/mL | hr | ng × hr/mL |
| 0.6 | 0.87 | 1 | 1.9 | 0.131 ± 0.027 | 4.25 ± 2.5 | 0.596 ± 0.24 |
| 1.2 | 1.7 | 2 | 3.8 | 0.165 ± 0.061 | 5.00 ± 2.4 | 0.918 ± 0.32 |
| 1.8 | 2.6 | 3 | 5.6 | 0.343 ± 0.18 | 5.50 ± 2.9 | 1.64 ± 0.80 |
| 6 | 8.7 | 10 | 19 | 0.438 ± 0.21 | 9.25 ± 3.4 | 3.05 ± 1.7 |

Lower limit of quantitation was 0.0500 ng/mL, except 0.87 μmol/kg dose was 0.0250 ng/mL

FIG. 3A and FIG. 3B compare mean plasma concentrations over time of hydromorphone release following PO administration of a single dose unit and of multiple dose units of a composition comprising prodrug Compound PC-5 and trypsin inhibitor Compound 109.

The results in Table 3A, Table 3B, FIG. 3A and FIG. 3B indicate that administration of multiple dose units (as exemplified by 2, 3 and 10 dose units of the 109-to-PC-5 (2.2-to 1) dose unit) results in a plasma hydromorphone concentration-time PK profile that was not dose proportional to the plasma hydromorphone concentration-time PK profile of the single dose unit. In addition, the PK profile of the multiple dose units was modified compared to the PK profile of the equivalent dosage of prodrug in the absence of trypsin inhibitor.

### Synthesis of Ketone-modified Opioid Prodrugs

### Example 5

### Synthesis of N,N-Bis(tert-butyl) N′-2-(chlorocarbonyl(methyl)amino)ethylcarbamate

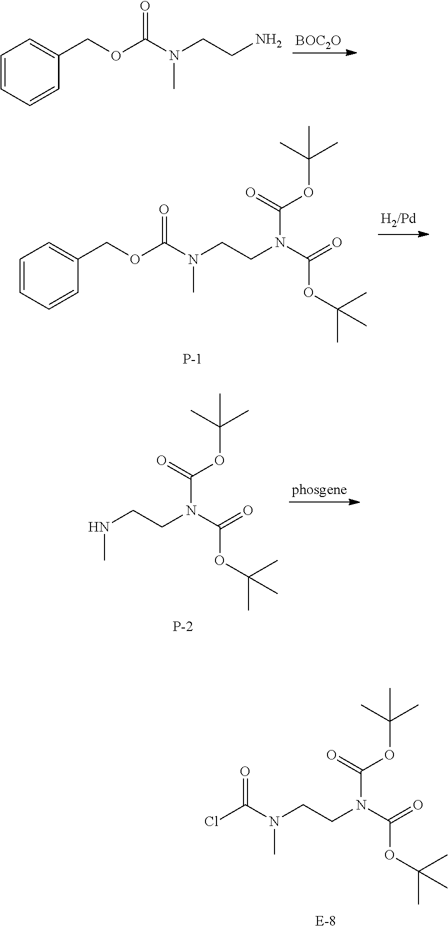

### Synthesis of [2-(Benzyloxycarbonyl-methyl-amino)-ethyl]-dicarbamic acid tert-butyl ester (P-1)

2-(Aminoethyl)-methyl-carbamic acid benzyl ester (2.0 g, 9.6 mmol) was dissolved in dichloroethene (DCE) (20 mL) at room temperature. Triethyl amine (NEW (1.40 mL, 11.5 mmol) was added, followed by di-tert-butyl dicarbonate (BOC₂O) (10.5 g, 48 mmol) and dimethylaminopyridine (DMAP) (120 mg). The reaction mixture was stirred at room temperature under nitrogen (N₂) for 2 h and then heated at 60° C. for 16 h. The reaction mixture was then concentrated. The residue was purified by silica gel chromatography, using 4/1 hexanes/EtOAc, to give P-1 in 86% yield (3.4 g, 8.3 mmol). MS: (m/z) calc: 408.2, observed (M+Na⁺) 431.9.

### Synthesis of N1, N1-bis-BOC-N2-methylethane-1,2-diamine (P-2)

P-1 (1.3 g, 3.18 mmol) was dissolved in methanol/EtOAc (10 mL/3 mL respectively). The mixture was degassed and saturated with N₂. Palladium on carbon (Pd/C) (330 mg, 5% on carbon) was added. The mixture was shaken in a Parr hydrogenator flask (50 psi H₂) for 4 h. The mixture was then filtered through a celite pad and the filtrate was concentrated to give P-2 (1.08 g, yield exceeded quantative). P-2 was used without further purification.

### Synthesis of N,N-Bis(tert-butyl)N′-2-(chlorocarbonyl(methyl)amino)ethylcarbamate (E-8)

P-2 (500 mg, 1.82 mmol) and NEt₃ (0.4 mL, 2.74 mmol) was mixed together in dichloromethane (4 mL). The mixture was added to a pre-chilled to 0° C. solution of phosgene (5.5 mL, 0.5 M in toluene). The reaction mixture was stirred at 0° C. for 1 h, followed by dilution with ether (20 mL) and filtered through filter paper. The filtrate was concentrated and passed through a short silica gel column (10 cm×3 cm), eluted with 3/1 hexanes/EtOAc. The fractions were concentrated to give N,N-Bis(tert-butyl) N′-2-(chlorocarbonyl(methyl)amino)ethylcarbamate (E-8) as a colorless solid in quantative yield (615 mg, 1.82 mmol). MS: (m/z) calc: 336.1, observed (M+Na⁺) 359.8.

### Example 6

### Synthesis of Oxycodone 6-(N-methyl-N-(2-amino)ethylcarbamate-2TFA

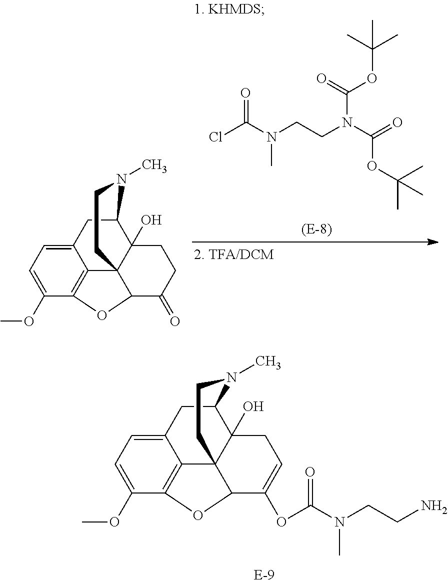

### Synthesis of oxycodone 6-(N-methyl-N-(2-amino)ethylcarbamate-2TFA (E-9)

Oxycodone free base (6.5 g, 20.6 mmol) was dissolved in dry, degassed tetrahydrofuran (120 mL), and the mixture was cooled to −10° C. using dry ice/acetone bath. Potassium bis(trimethylsilyl)amide (KHMDS) (103.0 mL, 51.6 mmol, 0.5 M in toluene) was added via cannula. The mixture was stirred under N₂ at below −5° C. for 30 min. N,N-Bis(tert-butyl) N′-2-(chlorocarbonyl(methyl)amino)ethylcarbamate (8.0 g, 23.7 mmol), (E-8) prepared as described in the Examples herein, in THF (30 mL) was then added via cannula over 15 min. The mixture was stirred at −5° C. for 30 min. Another portion of carbamoyl chloride (4.0 g, 11.9 mmol) in THF (10 mL) was added. The reaction was stirred at room temperature for 2 h. Sodium bicarbonate (10 mL, sat. aq.) was added. The mixture was concentrated in vacuo to half of its initial volume. EtOAc (50 mL) was added and layers were separated. The organic phase was further washed with water (3×20 mL), brine (40 mL) and then was concentrated. The residue was purified by silica gel chromatography, using DCM/MeOH (gradient 100/1 to 100/15) to afford a white foam in 55% yield (7.0 g, 13.4 mmol). This material was dissolved in a 1:1 mixture of DCM/trifluoroacetic acid (TFA) (20 mL/20 mL) at room temperature and stirred for 1 h. The solution was then concentrated in vacuo to afford oxycodone 6-(N-methyl-N-(2-amino)ethylcarbamate-2TFA as a thick oil (7.3 g, 11.4 mmol, 99% purity). MS: (m/z) calc: 415.2, observed (M+H⁺) 416.5. The oxycodone 6-(N-methyl-N-(2-amino)ethylcarbamate-2TFA (E-9) was used without further purification.

### Example 7

### Synthesis of Oxycodone 6-(N-methyl-N-(2-N′-acetylarginylamino)) ethylcarbamate (Compound KC-2)

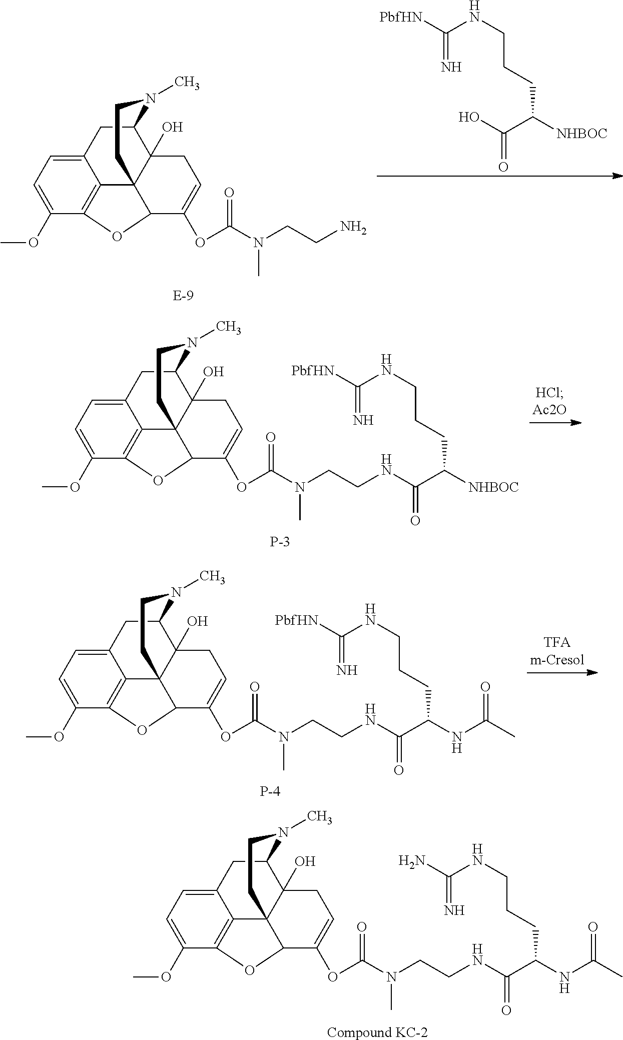

### Synthesis of oxycodone 6-(N-methyl-N-(2-N′-Boc-arginyl(Pbf)amino))ethylcarbamate (P-3)

Oxycodone 6-(N-methyl-N-(2-amino)ethylcarbamate-2TFA (7.3 g, 11.4 mmol), (E-9) prepared as described in the Example herein, was dissolved in dimethylformamide (DMF) (60 mL). Boc-Arg(Pbf)-OH (6.0 g, 11.4 mmol), HATU (4.75 g, 12.5 mmol) and diisopropylethylamine (DIPEA) (6.0 mL, 34.4 mmol) were added in this order. The reaction was stirred at room temperature for 2 h. The mixture was then concentrated in vacuo and the residue was partitioned between EtOAc/water (100 mL/'60 mL). The organic layer was washed with water (60 mL), brine (50 mL), dried over Na₂SO₄ and concentrated to afford crude P-3 (11.0 g). P-3 was used without further purification.

### Synthesis of oxycodone 6-(N-methyl-N-(2-N′-acetylarginyl(Pbf)amino))ethylcarbamate (P-4)

P-3 (11.0 g), prepared as described above, was dissolved into dioxane (10 mL) and cooled to 0° C. A hydrochloric acid (HCl) solution in dioxane (4 N, 30 mL) was added. The mixture was stirred at room temperature for 3 h and then concentrated in vacuo. 10 g of the crude mixture was dissolved in a mixture of DIPEA (5.0 mL 28.5 mmol) in DCM (60 mL). Acetic anhydride (1.4 mL, 14.3 mmol) was added drop wise. The reaction mixture was stirred at room temperature for 2 h. NaHCO₃ (30 mL, sat. aq.) was then added. The layers were separated and the DCM layer was dried over Na₂SO₄, filtered and concentrated to afford P-4 (8.5 g). P-4 was used without further purification.

### Synthesis of oxycodone 6-(N-methyl-N-(2-N′-acetylarginylamino))ethylcarbamate, as the bis-TFA salt (Compound KC-2)

P-4 (8.5 g) was dissolved in a mixture of m-cresol (3 mL) in TFA (30 mL). The mixture was stirred at room temperature for 3 h. TFA was then removed in vacuo. The residue was dissolved into MeOH (10 mL) and added drop wise to a stirred HCl solution in ether (40 mL, 2 M). The white solid was filtered and washed with ethyl ether (4×30 mL). The white solid was further purified by prep HPLC (*RP-18e C18 column (4.6×50 mm); flow rate 1.5 mL/min; mobile phase A: 0.1% TFA/water; mobile phase B 0.1% TFA/acetonitrile (CH₃CN); gradient elution), yielding Compound KC-2 (3.5 g, 4.1 mmol, 96.6% purity). MS: (m/z) calc: 613.7, observed (M+H⁺) 614.5.

### Example 8

### Synthesis of N-{(S)-4-guanidino-1-[2-(methyl-[(5R,9R,13S,14S)-4,5a-epoxy-6,7-didehydro-14-hydroxy-3-methoxy-17-methylmorphinan-6-oxy]carbonyl-amino)-ethylcarbamoyl]-butyl}-malonamic acid (Compound KC-3)

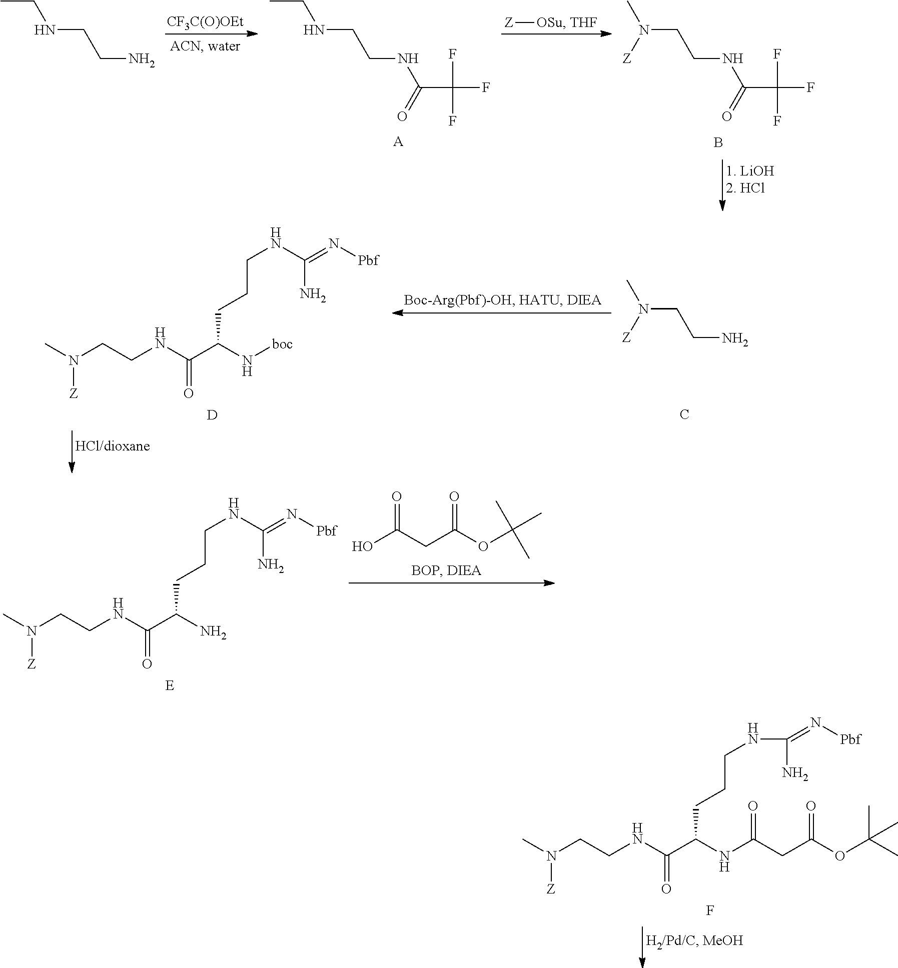

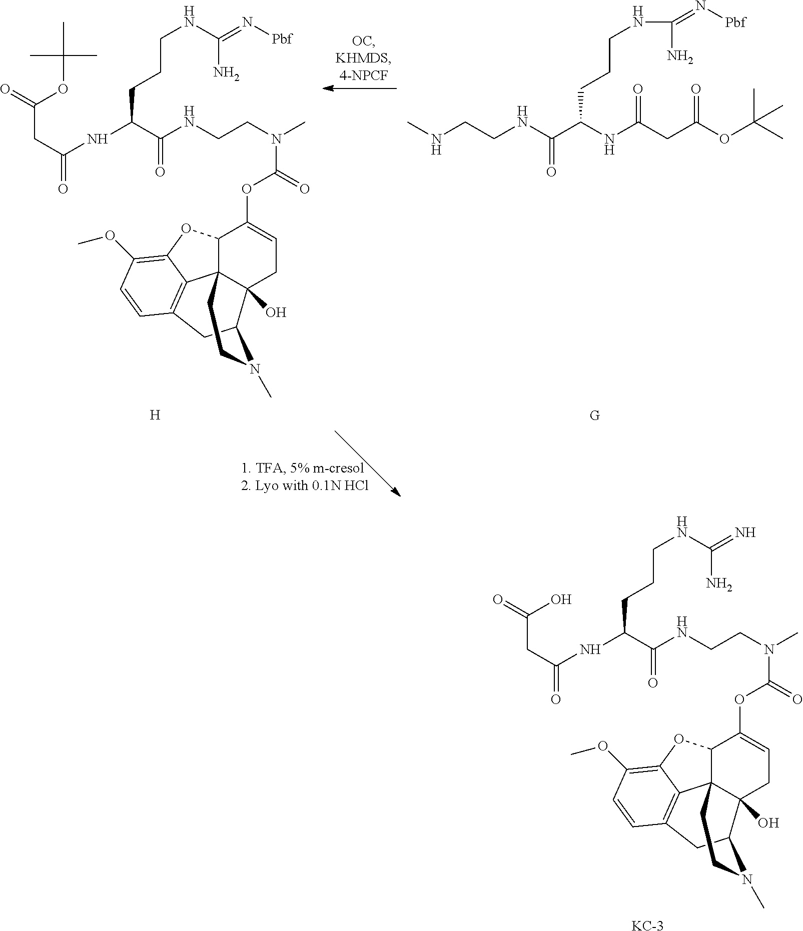

### Synthesis of 2,2,2-trifluoro-N-(2-methylamino-ethyl)-acetamide (A)

A solution of N-methylethylenediamine (27.0 g, 364 mmol) and ethyl trifluoroacetate (96.6 mL, 812 mmol) in a mixture of ACN (350 mL) and water (7.8 mL, 436 mmol) was refluxed with stirring overnight. Solvents were evaporated in vacuo. The residue was re-evaporated with i-PrOH (3×100 mL), followed by heat-cool crystallization from DCM (500 mL). Formed crystals were filtered, washed with DCM and dried in vacuo to provide compound A (88.3 g, 85%) as white solid powder.

### Synthesis of methyl-[2-(2,2,2-trifluoro-acetylamino)-ethyl]carbamic acid benzyl ester (B)

A solution of compound A (88.2 g, 311 mmol) and DIEA (54.1 mL, 311 mmol) in THF (350 mL) was cooled in an ice bath, followed by the addition of a solution of N-(benzyloxycarbonyl)succinimide (76.6 g, 307 mmol) in THF (150 mL) drop wise over the period of 20 min. The temperature of the reaction mixture was raised to ambient temperature and stirring was continued for an additional 30 min. Solvents were then evaporated and the resulting residue was dissolved in EtOAc (600 mL). The organic layer was extracted with 5% aq. NaHCO₃ (2×150 mL) and brine (150 mL). The organic layer was evaporated to provide compound Bas yellowish oil. LC-MS [M+H] 305.1 (C₁₃H₁₅F₃N₂O₃+H, calc: 305.3). Compound B was used directly in the next reaction without purification as a MeOH solution.

### Synthesis of (2-amino-ethyl)-methyl-carbamic acid benzyl ester (C)

To a solution of compound B (˜311 mmol) in MeOH (1.2 L) was added a solution of LiOH (14.9 g, 622 mmol) in water (120 mL). The reaction mixture was stirred at ambient temperature for 3 h. Solvents were evaporated to 75% of the initial volume followed by dilution with water (400 mL). The solution was extracted with EtOAc (2×300 mL). The organic layer was washed with brine (200 mL), dried over MgSO₄ and evaporated in vacuo. The residue was dissolved in ether (300 mL) and treated with 2 N HCl/ether (200 mL). Formed precipitate was filtrated, washed with ether and dried in vacuo to provide the hydrochloric salt of compound C (67.8 g, 89%) as a white solid. LC-MS [M+H] 209.0 (C₁₁H₁₆N₂O₂+H, calc: 209.3). Compound C was used directly in the next reaction without purification as a DMF solution.

### Synthesis of {2-[boc-Arg(Pbf)]-aminoethyl}-methyl-carbamic acid benzyl ester (D)

A solution of Boc-Arg(Pbf)-OH (16.0 g, ˜30.4 mmol), compound C hydrochloride (8.2 g, 33.4 mmol) and DIEA (16.9 mL, 97.2 mmol) in DMF (150 mL) was cooled in an ice bath followed by the addition of a solution of HATU (13.8 g, 36.4 mmol) drop wise over 20 min. The temperature of the reaction mixture was raised to ambient temperature and stirring was continued for an additional 1 h. The reaction mixture was diluted with EtOAc (1 L) and extracted with water (3×200 mL) and brine (200 mL). The organic layer was dried over MgSO₄ and evaporated to provide compound D (24.4 g, yield exceeded quantitative) as a yellowish oil. LC-MS [M+H] 717.4 (C₃₅H₅₂N₆O₈S+H, calc: 717.9). Compound D was used directly in the next reaction without purification as a dioxane solution.

### Synthesis of {2-[H-Arg(Pbf)]-aminoethyl}-methyl-carbamic acid benzyl ester (E). Compound D (24.4 g, ˜30.4 mmol) was dissolved in dioxane (150 mL) and treated with 4 N HCl/dioxane (150 mL, 600 mmol) at ambient temperature for 1 h. The solvent was then evaporated. The residue was suspended in i-PrOH (100 mL) and the mixture was evaporated (procedure was repeated twice). The residue was then dried in vacuo to provide compound E (21.1 g, yield exceeded quantitative) as a yellowish solid. LC-MS [M+H] 617.5 (C₃₀H₄₄N₆O₆S+H, calc: 617.8). Compound E was used directly in the next reaction without purification as a DMF solution.

### Synthesis of {2-[2-tert-butylmalonyl-Arg(Pbf)]-aminoethyl}methyl-carbamic acid benzyl ester (F)

A solution of compound E (21.1 g, ˜30.4 mmol), mono-tert-butyl malonate (5.9 mL, 36.7 mmol), BOP (16.2 g, 36.7 mmol) and DIEA (14.9 mL, 83.5 mmol) in DMF (100 mL) was maintained at ambient temperature for 1 h. The reaction mixture was diluted with EtOAc (1 L) and extracted with water (500 mL), 5% aq. NaHCO₃ (500 mL), water (3×500 mL) and brine (500 mL). The organic layer was dried over MgSO₄, filtered, and then evaporated to provide compound F (24.5 g, 97%) as a yellowish amorphous solid. LC-MS [M+H] 759.6 (C₃₇H₅₄N₆O₉S+H, calc: 759.9). Compound F was used without further purification.

### Synthesis of N-{2-[2-tert-butylmalonyl-Arg(Pfb)]}-N′-methyl-ethane-1,2-diamine (G)

Compound F (12.3 g, 16.7 mmol) was dissolved in methanol (100 mL) followed by the addition of a Pd/C (5% wt, 2.0 g) suspension in water (2 mL). The reaction mixture was subjected to hydrogenation (Parr apparatus, 70 psi H₂) at ambient temperature for 1 h. The catalyst was filtered and washed with methanol. The filtrate was evaporated in vacuo to provide compound G (10.0 g, 99%) as a colorless amorphous solid. LC-MS [M+H] 625.5 (C₂₉H₄₈N₆O₇S+H, calc: 625.8). Compound G was used without further purification.

### Oxycodone Free Base

Oxycodone hydrochloride (10.0 g, 28.5 mmol) was dissoleved in chloroform (150 mL) and washed with 5% aq. NaHCO₃ (50 mL). The organic layer was dried over MgSO₄ and evaporated. The residue was dried in vacuo overnight to provide oxycodone free base (8.3 g, 93%) as a white solid.

### Synthesis of N-{(S)-4-(2,2,4,6,7-pentamethyldihydrobenzofuran-5-sulfonyl-guanidino)-1-[2-(methyl-[(5R,9R,13S,14S)-4,5a-epoxy-6,7-didehydro-14-hydroxy-3-methoxy-17-methylmorphinan-6-oxy]carbonyl-amino)-ethylcarbamoyl]-butyl}-malonamic acid tert-butyl ester (H).

A solution of oxycodone free base (6.6 g, 21.0 mmol) in THF (400 mL) was cooled to −20° C., followed by addition of a 0.5 M solution of KHMDS in toluene (46.3 mL, 23.1 mmol). The obtained solution was then added to a solution of 4-nitro-phenyl chloroformate (4.3 g, 21.0 mmol) in THF (100 mL) drop wise over the period of 20 min at −20° C. The reaction was maintained at −20° C. for an additional 1 h, followed by addition of a solution of compound G (10.0 g, 16.1 mmol) in THF (200 mL) at −20° C. The reaction mixture was allowed to warm to ambient temperature and stirred overnight. Solvents were evaporated in vacuo. The resulting residue was dissolved in EtOAc (20 mL) and precipitated with ether (1 L). The formed precipitate was filtrated, washed with ether and dried in vacuo to provide compound H (13.6 g, 87%) as an off-white solid. LC-MS [M+H] 966.9 (C₄₈H₆₇N₇O₁₂S+H, calc: 966.2).

### Synthesis of N-{(S)-4-guanidino-1-[2-(methyl-[(5R,9R,13S,14S)-4,5a-epoxy-6,7-didehydro-14-hydroxy-3-methoxy-17-methylmorphinan-6-oxy]carbonyl-amino)-ethylcarbamoyl]-butyl}-malonamic acid (Compound KC-3)

Compound H (13.6 g, 14.1 mmol) was dissolved in a mixture of 5% m-cresol/TFA (100 mL). The reaction mixture was maintained at ambient temperature for 1 h, followed by dilution with ethyl ether (1 L). The formed precipitate was filtered, washed with ether and hexane, and dried in vacuo to provide a TFA salt of Compound KC-3 (11.4 g, 81%) as an off-white solid. LC-MS [M+H] 658.6 (C₃₁H₄₃N₇O₉+H, calc: 658.7).

The TFA salt of crude Compound KC-3 (11.4 g, 11.4 mmol) was dissolved in water (50 mL). The obtained solution was subjected to HPLC purification. [Nanosyn-Pack YMC-GEL-ODS A (100-10) C-18 column (75×500 mm); flow rate: 250 mL/min; injection volume 50 mL; mobile phase A: 100% water, 0.1% TFA; mobile phase B: 100% ACN, 0.1% TFA; isocratic elution at 0% B in 4 min, gradient elution from 0% to 10% B in 20 min, isocratic elution at 10% B in 30 min, gradient elution from 10% B to 30% B in 41 min; detection at 254 nm]. Fractions containing Compound KC-3 were combined and concentrated in vacuo. The TFA counterion of the latter was replaced with an HCl counterion via lyophilization using 0.1N HCl to provide a HCl salt of Compound KC-3 (4.2 g, 41% yield) as a white solid. LC-MS [M+H] 658.6 (C₃₁H₄₃N₇O₉+H, calc: 658.7).

### Biological Data of Ketone-Modified Opioid Prodrugs

### Example 9

### Oral Administration of Compound KC-2 Co-Dosed with Trypsin Inhibitor Compound 109 to Rats

Saline solutions of Compound KC-2 were dosed at 7.3 μmol/kg (5 mg/kg) and 73 μmol/kg (50 mg/kg). The higher dose was co-dosed with increasing concentrations of Compound 109 (Catalog No. 3081, Tocris Bioscience or Catalog No. WS38665, Waterstone Technology) as indicated in Table 24 via oral gavage into jugular vein-cannulated male Sprague Dawley rats (4 per group) that had been fasted for 16-18 hr prior to oral dosing. At specified time points, blood samples were drawn, harvested for plasma via centrifugation at 5,400 rpm at 4° C. for 5 min, and 100 microliters (μl) plasma transferred from each sample into a fresh tube containing 2 μl of 50% formic acid. The tubes were vortexed for 5-10 seconds, immediately placed in dry ice and then stored in −80° C. freezer until analysis by HPLC/MS.

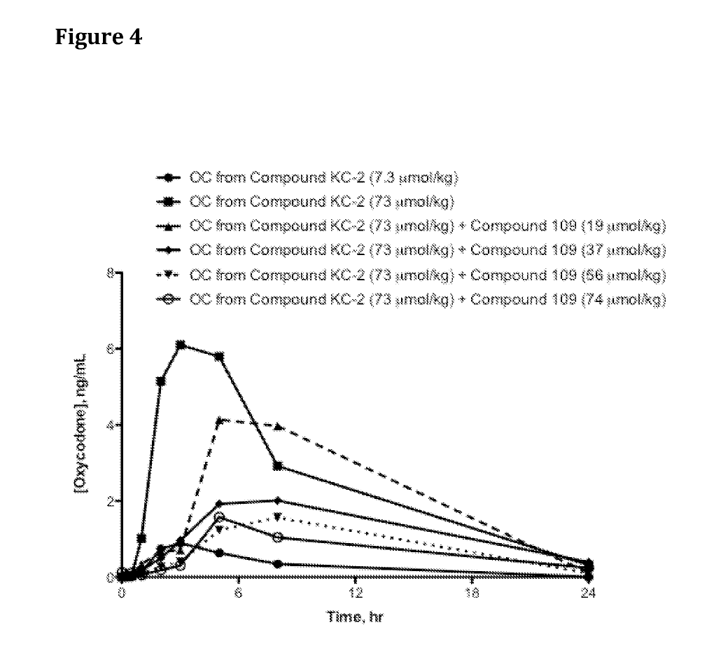

Table 4 and FIG. 4 provide oxycodone exposure results for rats administered with different doses of Compound KC-2. Results in Table 4 are reported, for each group of rats, as (a) maximum plasma concentration (Cmax) of oxycodone (OC) (average+standard deviation), (b) time after administration of Compound KC-2 to reach maximum oxycodone concentration (Tmax) (average±standard deviation) and (c) area under the curve (AUC) from 0 to 24 hr (average±standard deviation).

TABLE 4
Rat dosing PO with Compound KC-2 in the absence or presence of Compound 109

| KC-2 | KC-2 | Compound | Compound | | | |
|---|---|---|---|---|---|---|
| Dose, | Dose, | 109 Dose, | 109 Dose, | OC Cmax ± | Tmax ± sd, | AUC ± sd, |
| mg/kg | μmol/kg | mg/kg | μmol/kg | sd, ng/mL | hr | ng * hr/mL |
| 5 | 7.3 | 0 | 0 | 0.918 ± 0.30 | 2.75 ± 0.5 | 4.30 ± 1.1 |
| 50 | 73 | 0 | 0 | 7.03 ± 2.3 | 3.75 ± 1.5 | 59.9 ± 14 |
| 50 | 73 | 10 | 19 | 4.44 ± 1.5 | 6.50 ± 1.7 | 51.0 ± 16 |
| 50 | 73 | 20 | 37 | 2.25 ± 0.89 | 7.25 ± 1.5 | 29.2 ± 8.9 |
| 50 | 73 | 30 | 56 | 1.77 ± 0.57 | 6.50 ± 1.7 | 19.8 ± 7.6 |
| 50 | 73 | 40 | 74 | 1.64 ± 0.96 | 5.75 ± 1.5 | 16.5 ± 5.9 |

Lower limit of quantitations were 0.0250 ng/ml

FIG. 4 compares mean plasma concentrations over time of oxycodone release following PO administration of Compound KC-2 with increasing amounts of co-dosed trypsin inhibitor Compound 109.

The results in Table 4 and FIG. 4 indicate Compound 109's ability to attenuate Compound KC-2's ability to release oxycodone in a dose dependent manner, both by suppressing Cmax and AUC and by delaying Tmax.

### Example 10

### Oral Administration of Compound KC-3 and Trypsin Inhibitor Compound 109 to Rats

This Example demonstrates the ability of a trypsin inhibitor of the embodiments to affect drug release into plasma from Compound KC-3 administered orally.

Saline solutions of Compound KC-3 (which can be prepared as described in the Examples herein) were dosed at 6.8 μmol/kg (5 mg/kg) and 68 μmol/kg (50 mg/kg) Compound KC-3 with or without a co-dose of increasing concentrations of Compound 109 (Catalog No. 3081, Tocris Bioscience or Catalog No. WS38665, Waterstone Technology) as indicated in Table 5 via oral gavage into jugular vein-cannulated male Sprague Dawley rats (4 per groups) that had been fasted for 16-18 hr prior to oral dosing. At specified time points, blood samples were drawn, harvested for plasma via centrifugation at 5,400 rpm at 4° C. for 5 min, and 100 μl plasma transferred from each sample into a fresh tube containing 2 μA of 50% formic acid. The tubes were vortexed for 5-10 seconds, immediately placed in dry ice and then stored in −80° C. freezer until analysis by HPLC/MS.

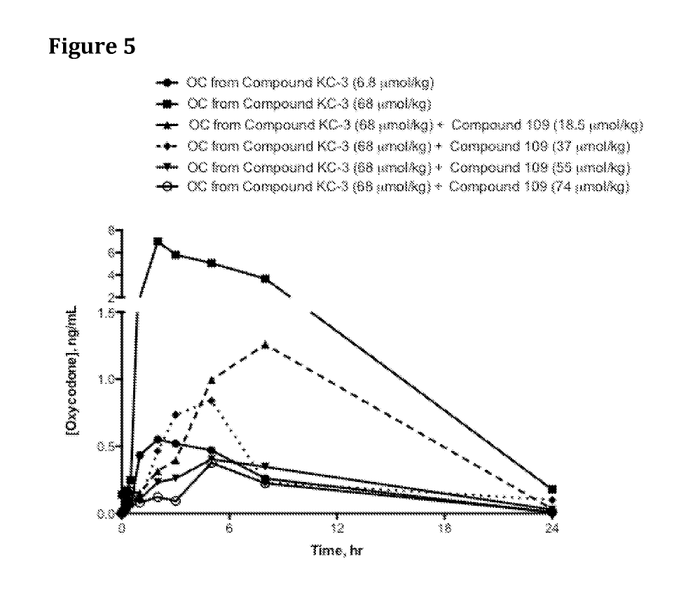

Table 5 and FIG. 5 provide oxycodone exposure results for rats administered with Compound KC-3 in the absence or presence of trypsin inhibitor. Results in Table 5 are reported as (a) maximum plasma concentration (Cmax) of oxycodone (OC) (average±standard deviation), (b) time after administration of Compound KC-3 to reach maximum oxycodone concentration (Tmax) (average±standard deviation) and (c) area under the curve (AUC) from 0 to 24 hr (average±standard deviation).

TABLE 5
Cmax, Tmax and AUC values of oxycodone in rat plasma

| KC-3 | KC-3 | Compound | Compound | | | AUC ± sd |
|---|---|---|---|---|---|---|
| Dose, | Dose, | 109 Dose, | 109 Dose, | OC Cmax ± | Tmax ± | (ng × |
| mg/kg | μmol/kg | mg/kg | μmol/kg | sd, ng/mL | sd, hr | hr)/mL |
| 5 | 6.8 | 0 | 0 | 0.611 ± 0.10 | 3.00 ± 1.4 | 3.95 ± 1.6 |
| 50 | 68 | 0 | 0 | 7.08 ± 2.6 | 3.00 ± 1.4 | 59.1 ± 23 |
| 50 | 68 | 10 | 18.5 | 1.26 ± 0.34 | 8.00 ± 0.0 | 12.3 ± 2.9 |
| 50 | 68 | 20 | 37 | 1.05 ± 0.61 | 3.75 ± 1.5 | 10.5 ± 5.4 |
| 50 | 68 | 30 | 55 | 0.49 ± 0.19 | 4.50 ± 2.6 | 2.82 ± 1.3 |
| 50 | 68 | 40 | 74 | 0.47 ± 0.36 | 4.63 ± 3.1 | 2.71 ± 3.7 |

Lower limit of quantitation was 0.025 ng/mL

FIG. 5 compares mean plasma concentrations over time of oxycodone release following PO administration of Compound KC-3 with or without a co-dose of trypsin inhibitor.

The results in Table 5 and FIG. 5 indicate that Compound 109 attenuates Compound KC-3's ability to release oxycodone, both by suppressing Cmax and AUC and by delaying Tmax.

### Synthesis of Ketone-Modified Opioid Prodrugs

### Example 11

### Synthesis of [(S)-1-(oxycodone-6-enol-carbonyl-methyl-amino)-1-carbonyl-sarcosine]ethylamine (Compound KC-23)

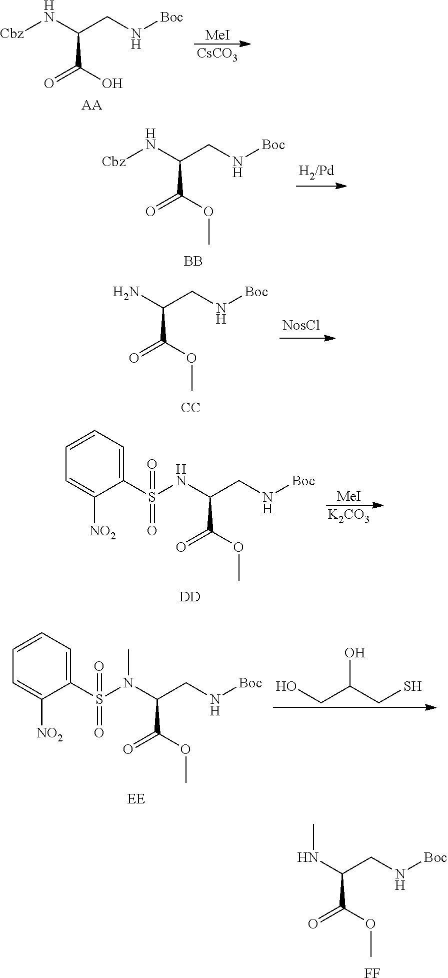

### Preparation of Compound BB

A solution of (S)-2-Benzyloxycarbonylamino-3-tert-butoxycarbonylamino-propionic acid (Compound AA) (30.0 g, 88.8 mmol) in DMF (100 mL) was cooled down to 0° C., followed by the addition of CsCO₃ (28.9 g, 88.8 mmol). The reaction was stirred for 5 min, followed by the dropwise addition of MeI (6.6 mL, 106.6 mmol). The reaction was allowed to rise to ambient temperature, and then was stirred for 1 h. Additional amounts of MeI (6.6 mL, 106.6 mmol, each) were then added after 30 min and 60 min respectively. The reaction mixture was then stirred for 1 h at ambient temperature. Solvents were removed in vacuo, and the residue was dissolved in EtOAc (800 mL), and washed with water (3×300 mL) and brine (300 mL). The organic layer was separated and dried over MgSO₄. The solvent was removed in vacuo to afford crude Compound BB in 94% yield (29.6 g, 83.8 mmol) as an amorphous solid. LC-MS [M+H] 353.0 (C₁₇H₂₄N₂O₆+H, calc: 353.4). Compound BB was used directly in the next reaction without further purification.

### Preparation of Compound CC

A solution of Compound BB (29.6 g, 83.8° mmol) in MeOH (500 mL) was treated with palladium (5 wt. % on activated carbon, 100 mg) suspended in water (5 mL), and subjected to hydrogenation at 70 psi for 2 h. The reaction mixture was then filtered using a celite pad, and the removal of MeOH in vacuo yielded Compound CC, yield exceeded quantitative, (18.5 g, 83.8 mmol) as a colorless oil. LC-MS [M+H] 219.0 (C₉H₁₈N₂O₄+H, calc: 219.3). Compound CC was used directly in the next reaction without further purification.

### Preparation of Compound DD

A mixture of Compound CC (18.5 g, 83.8 mmol) and TEA (15.1 mL, 108.9 mmol) in DCM (200 mL) was cooled down to 0° C., followed by the dropwise addition of NosCl (20.5 g, 92.2 mmol) solution in THF (100 mL). The reaction was stirred for 1 h at 0° C. The reaction mixture was then allowed to rise to ambient temperature. Solvents were then removed in vacuo, and the residue was dissolved in EtOAc (800 mL), and washed with water (4×200 mL) and brine (200 mL). The organic layer was separated and dried over MgSO₄. The solvent was removed in vacuo to afford crude Compound DD in 99% yield (33.3 g, 82.6 mmol) as a white solid. LC-MS [M+H] 403.7 (C₁₅H₂₁N₃O₈S+H, calc: 404.4). Compound DD was used directly in the next reaction without further purification.

### Preparation of Compound EE

A solution of Compound DD (33.3 g, 82.6 mmol) in DMF (150 mL) was cooled down to 0° C., followed by the addition of K₂CO₃ (57.0 g, 412.9 mmol). The reaction was stirred for 5 min, followed by the dropwise addition of MeI (15.4 mL, 247.7 mmol). The reaction mixture was allowed to rise to ambient temperature, and stirred for 1 h. K₂CO₃ was then filtered off, and the resulting solution was condensed in vacuo. The residue was then dissolved in EtOAc (800 mL), and washed with water (3×200 mL) and brine (200 mL). The organic layer was separated and dried over MgSO₄. The solvent was removed in vacuo to afford crude Compound EE in 97% yield (33.6 g, 80.58 mmol) as an amorphous solid. LC-MS [M+H] 418.4 (C₁₆H₂₃N₃O₈S+H, calc: 418.4). Compound EE was used directly in the next reaction without further purification.

### Preparation of Compound FF

To a solution of Compound EE (2.3 g, 5.5 mmol) in DMF (25 mL) at ambient temperature was added K₂CO₃ (7.6 g, 55.1 mmol) followed by thioglycerol (4.8 mL, 55.1 mmol). The reaction mixture was stirred at ambient temperature for 16 h. The reaction mixture was then filtered and DMF was removed in vacuo. The residue was taken into EtOAc (300 mL), and washed with water (2×200 mL) and brine (200 mL). The organic layer was separated, dried over Na₂SO₄, and removal of solvent in vacuo afforded crude Compound FF. Crude Compound FF was purified by flash chromatography using 1:1 EtOAc/Hexane and afforded Compound FF in 58% yield (5 steps) (0.74 g, 3.2 mmol) as an oil. LC-MS [M+H] 233.4 (C₁₀H₂₀N₂O₄+H, calc: 233.1).

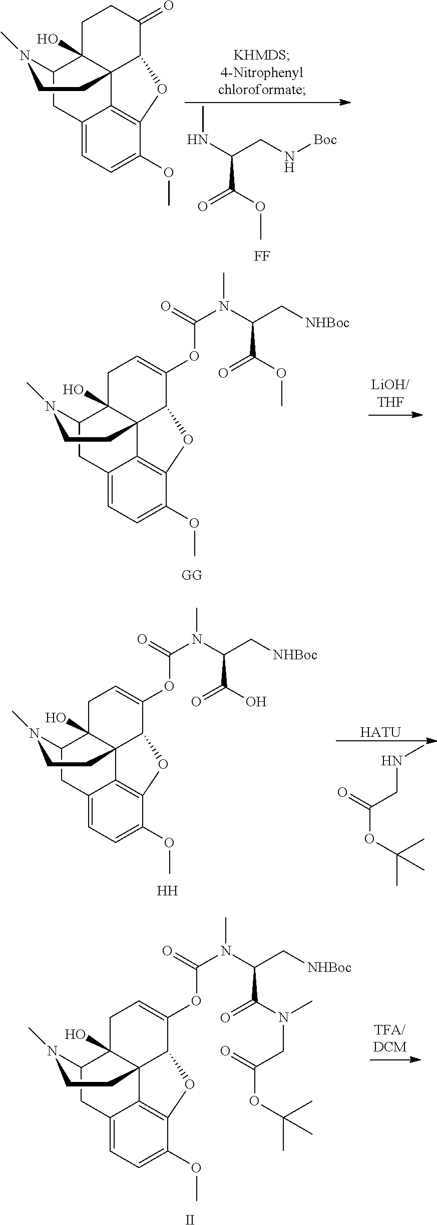

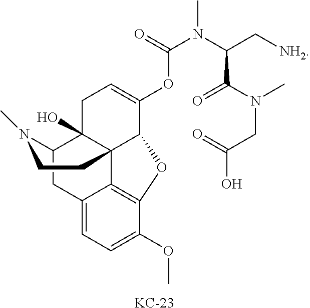

### Preparation of Compound GG

To a solution of oxycodone (1.1 g, 3.5 mmol) in THF (20 mL) at −60° C. was added a KHMDS solution (0.5 M in toluene, 7.61 mL) dropwise. After stirring at this temperature for 10 min, this reaction mixture was added to a solution of 4-Nitrophenyl chloroformate (698 mg, 3.5 mmol) in THF (15 mL) at −60° C. The reaction mixture was stirred for 30 min, a solution of Compound FF (0.67 g, 2.9 mmol) was added as a THF solution (3 mL), and the reaction was stirred at ambient temperature for 18 h. The reaction was then concentrated in vacuo, and the residue diluted with EtOAC (100 mL). The mixture was then washed with water (2×75 mL) and brine (50 mL). The organic layer was separated, dried over Na₂SO₄, and filtered; removal of solvents in vacuo afforded the crude Compound GG. Crude Compound GG was dissolved in water (15 mL), and the solution was subjected to HPLC purification. [Nanosyn-Pack Microsorb (100-10) C-18 column (50×300 mm); flow rate: 100 mL/min; injection volume 15 mL; mobile phase A: 100% water, 0.1% TFA; mobile phase B: 100% ACN, 0.1% TFA; isocratic elution at 0% B in 5 min, gradient elution from 0% to 20% B in 20 min, isocratic elution at 20% B in 20 min, gradient elution from 20% B to 45% B in 40 min; detection at 254 nm]. Fractions containing the desired compound were combined and concentrated in vacuo to provide Compound GG in 12% yield (220 mg, 0.32 mmol) as a white solid. LC-MS [M+H] 574.2 (C₂₉H₃₉N₃O₉+H, calc: 574.3).

### Preparation of Compound HH

To a solution of Compound GG (234 mg, 0.408 mmol) in THF (5 mL) was added 1M aqueous LiOH (1.2 mL, 1.2 mmol); the reaction mixture was stirred at ambient temperature for 1 h. The reaction mixture was diluted with water (50 mL), and the pH was adjusted to pH 5 with saturated aqueous NaHCO₃. Most of the water was then removed in vacuo until about 15 mL remained; this solution was subjected to HPLC purification. [Nanosyn-Pack Microsorb (100-10) C-18 column (50×300 mm); flow rate: 100 mL/min; injection volume 15 mL; mobile phase A: 100% water, 0.1% TFA; mobile phase B: 100% ACN, 0.1% TFA; isocratic elution at 0% B in 5 min, gradient elution from 0% to 20% B in 20 min, isocratic elution at 20% B in 20 min, gradient elution from 20% B to 45% B in 40 min; detection at 254 nm]. Fractions containing the desired compound were combined and concentrated in vacuo. The residue was dissolved in ACN (˜2 mL) and 0.1 N HCl (˜8 mL), and lyophilized overnight to provide Compound HH 96% yield (220 mg, 0.39 mmol) as a white solid. LC-MS [M+H] 560.3 (C₂₈H₃₇N₃O₉+H, calc: 560.25).

### Preparation of Compound II

To a solution of Compound HH (220 mg, 0.33 mmol), methylamino-acetic acid tert-butyl ester (237 mg, 1.64 mmol) and DIEA (5.0 mL, 47.4 mmol) in DMF (10 mL) at 5° C. was added HATU (124.1 mg, 0.33 mmol) in portions. The reaction mixture was raised to ambient temperature, and stirring was continued for an additional 1 h. Solvents were removed in vacuo, and the reaction mixture was diluted with EtOAc (100 mL), and washed with water (2×100 mL) and brine (50 mL). The organic layer was separated, dried over Na₂SO₄, and filtered; removal of solvents in vacuo afforded crude Compound II, yield exceeded quantitative, (226 mg, 0.33 mmol) as a foamy solid. LC-MS [M+H] 687.4 (C₃₅H₅₀N₄O₁₀+H, calc: 687.4). Compound II was used directly in the next reaction without further purification.

### Preparation of [(S)-1-(oxycodone-6-enol-carbonyl-methyl-amino)-1-carbonyl-sarcosine]ethylamine (Compound KC-23)

Compound II (226 mg, 0.33 mmol) was treated with 30% TFA in DCM (10 mL) for 1 h. The product was then precipitated via addition of Et₂O (100 mL). The precipitate was washed with Et₂O (2×50 mL) and dried in vacuo to afford Compound KC-23 as a TFA salt. The precipitate was dissolved in ACN (˜2 mL) and 0.1 N aqueous HCl (˜8 mL), and lyophilized overnight to provide the hydrochloride salt of Compound KC-23 in 83% yield (2 steps) (170 g, 0.27 mmol, 93.6% purity) as a white solid. LC-MS [M+H] 531.5 (C₂₆H₃₄N₄O₈+H, calc: 531.2).

### Example 12

### Synthesis of N-1-[(S)-2-(oxycodone-6-enol-carbonyl-methyl-amino)-2-carbonyl-sarcosine-ethyl amine]-arginine-glycine-acetate (Compound KC-7)

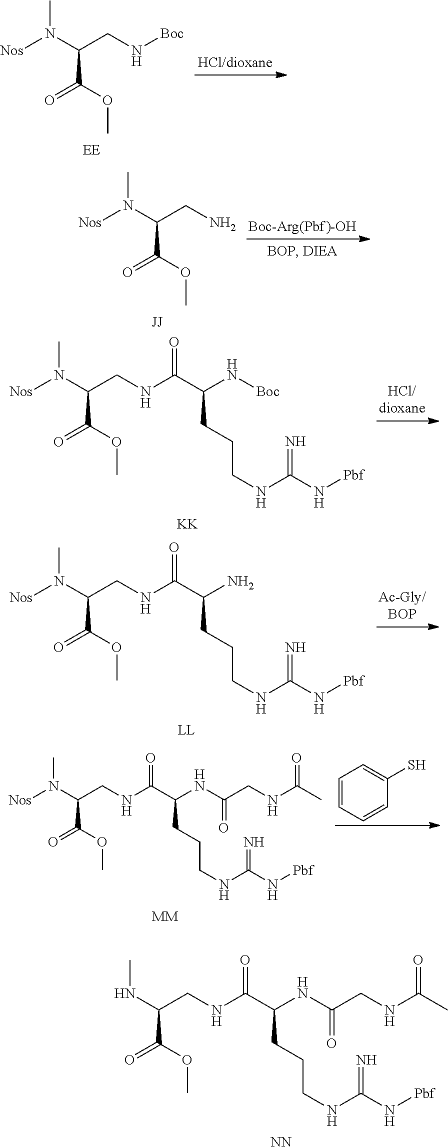

### Preparation of Compound JJ

A solution of Compound EE (12.2 g, 29.2 mmol), which can be prepared as described in Example 12, in dioxane (50 mL) was treated with HCl (4.0 M solution in 1,4-dioxane, 50 mL) for 1 h. The solvents were then removed in vacuo, until a volume of ˜10 mL remained, after which Et₂O (500 mL) was added. The resulting precipitate was filtered off, washed with Et₂O (2×100 mL), and dried to afford hydrochloric salt of Compound JJ, yield exceeded quantitative, (10.5 g, 29.2 mmol) as a white solid. LC-MS [M+H] 318.1 (C₁₁H₁₅N₃O₆S+H, calc: 318.3). Compound JJ was used directly in the next reaction without further purification.

### Preparation of Compound KK

A solution of Boc-Arg(Pbf)-OH (37.1 g, 70.5 mmol), Compound JJ (26.2 g, 74.2 mmol), and DIEA (64.5 mL, 371.0 mmol) in DMF (200 mL) was cooled to 0° C., followed by the addition of BOP (36.1 g, 81.6 mmol). The reaction mixture was then raised to ambient temperature, and stirring was continued for an additional 45 min. The reaction mixture was diluted with EtOAc (1 L), and extracted with water (3×200 mL) and brine (200 mL). The organic layer was separated and dried over MgSO₄. The solvent was removed in vacuo to afford crude Compound KK, yield exceeded quantitative, (77.0 g, 70.5 mmol) as an amorphous solid. LC-MS [M+H] 826.6 (C₃₅H₅₁N₇O₁₂S₂+H, calc: 827.0).

### Preparation of Compound LL

A solution of Compound KK (77.0 g, 70.5 mmol) in dioxane (200 mL) was treated with HCl (4.0 M solution in 1,4-dioxane, 200 mL) for 1 h. The solvents were then removed in vacuo, until a volume of ˜20 mL remained, after which Et₂O (1 L) was added. The resulting precipitate was filtered off, washed with Et₂O (2×200 mL), and dried to afford a hydrochloric salt of Compound LL, yield exceeded quantitative, (69.3 g, 70.5 mmol) as a white solid. LC-MS [M+H] 726.8 (C₃₀H₄₃N₇O₁₀S₂+H, calc: 726.9). Compound LL was used directly in the next reaction without further purification.

### Preparation of Compound MM

To a solution of Compound LL (69.3 g, 70.5 mmol), Ac-Gly-OH (9.5 g, 81.6 mmol), and DIEA (71.0 mL, 407.9 mmol) in DMF (400 mL) at 0° C. was added BOP (39.3 g, 89.0 mmol) in portions over 10 min. The reaction mixture was allowed to warm to ambient temperature and was stirred for 35 min. DMF was then removed in vacuo, and the residue was diluted with EtOAc (1 L). The solution was extracted with water (3×600 mL) and brine (600 mL). The organic layer was dried over MgSO₄ and filtered. The solvent was removed in vacuo to afford crude Compound MM, yield exceeded quantitative, (66.5 g, 70.45 mmol) as an amorphous solid. LC-MS [M+H] 825.7 (C₃₄H₄₈N₈O₁₂S₂+H, calc: 825.9). Compound MM was used directly in the next reaction without further purification.

### Preparation of Compound NN

A solution of Compound MM (22.2 g, 25 mmol) in DMF (50 mL) was treated with K₂CO₃ (9.7 g, 70 mmol) and thiophenol (7.2 mL, 70 mmol) at ambient temperature for 2.5 h. The reaction mixture was then filtered using a celite pad. The filtrate was evaporated in vacuo, and the residual oil was diluted with EtOAc (350 mL) and Et₂O (2 L) sequentially. The resulting precipitate was filtered off, washed with Et₂O (2×300 mL) and hexane (300 mL), and concentrated in vacuo to afford crude compound NN in 79% yield (12.4 g, 19.4 mmol) as a glass like solid. LC-MS [M+H] 640.5 (C₂₈H₄₅N₇O₈S+H, calc: 640.8). Compound NN was used directly in the next reaction without further purification.

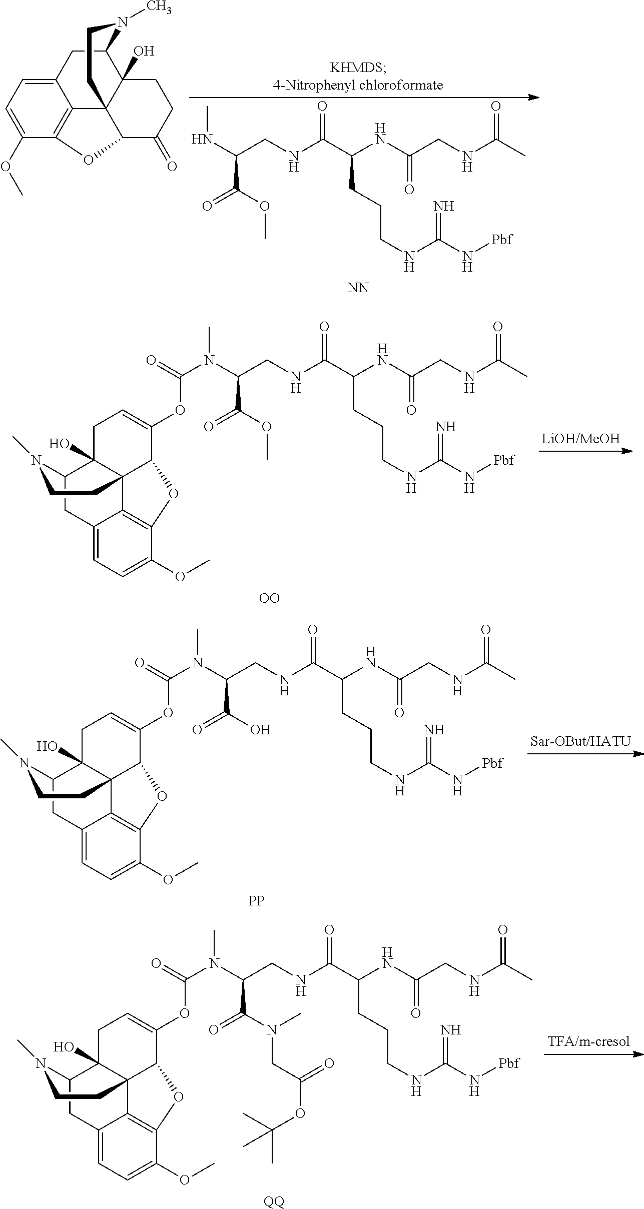

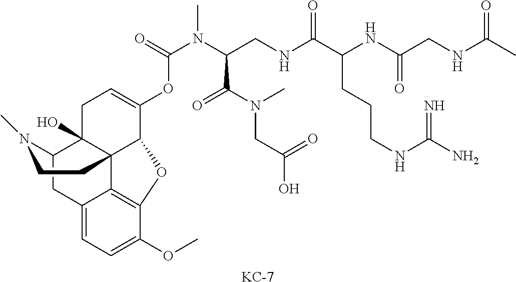

### Preparation of Compound OO

To a solution of oxycodone free base (5.1 g, 16.2 mmol) in anhydrous THF (200 mL) was added KHMDS (0.5M, in toluene, 35.0 mL, 17.7 mmol) dropwise over the period of 30 min at −20° C. The reaction mixture was stirred at −20° C. for 30 additional minutes. The obtained solution was added to a solution of 4-nitrophenyl chloroformate (3.0 g, 14.7 mmol) in anhydrous THF (200 mL) dropwise over the period of 30 min at −20° C. The reaction mixture was stirred at −20° C. for 30 additional minutes. To this solution was added Compound NN (5.0 g, 7.8 mmol) in THF/DMF (100 mL/4 mL) dropwise over the period of 30 min at −20° C. The reaction mixture was allowed to warm to ambient temperature. Solvents were removed in vacuo and the obtained viscous oil was left at ambient temperature overnight. The resultant oil was dissolved in DMSO (40 mL) and subjected to HPLC purification. [Nanosyn-Pack YMC-ODS-A (100-10) C-18 column (75×500 mm); flow rate: 250 mL/min; injection volume 5 mL×8; mobile phase A: 100% water, 0.1% TFA; mobile phase B: 100% ACN, 0.1% TFA; isocratic elution at 20% B in 12 min, gradient elution from 20% B to 32% B in 24 min, isocratic elution at 32% B in 20 min, gradient elution from 32% B to 52% B in 39 min; detection at 254 nm]. Fractions containing the desired product were combined and concentrated in vacuo to one-half of the initial volume, basified with 5% NaHCO₃ to pH 8.0, and extracted with DCM (3×300 mL). The organic layer was dried over MgSO₄, filtered, and the solvent was removed in vacuo to afford Compound OO in 37% yield (2.9 g, 2.9 mmol) as a yellowish solid. LC-MS [M+H] 982.3 (C₄₇H₆₄N₈O₁₃S+H, calc: 982.1).

### Preparation of Compound PP

A solution of Compound OO (2.9 g, 2.9 mmol) in MeOH (10 mL) was treated with a solution of LiOH (209 mg, 8.75 mmol) in water (10 mL) at ambient temperature for 20 min. The pH of the reaction mixture was adjusted to pH 5 with AcOH at ambient temperature. The obtained solution was evaporated to two-thirds of the initial volume and subjected to HPLC purification. [Nanosyn-Pack YMC-ODS-A (100-10) C-18 column (75×500 mm); flow rate: 250 mL/min; injection volume 7 mL; mobile phase A: 100% water, 0.1% TFA; mobile phase B: 100% ACN, 0.1% TFA; isocratic elution at 0% B in 4 min, gradient elution from 0% B to 90% B in 60 min, detection at 254 nm]. Fractions containing the desired product were combined and evaporated in vacuo to afford Compound PP in 51% yield (1.4 g, 1.5 mmol) as a white solid. LC-MS [M+H] 968.1 (C₄₆H₆₂N₈O₁₃S+H, calc: 968.1).

### Preparation of Compound QQ

To a solution of H-Sar-OtBu hydrochloric salt (379 mg, 2.1 mmol), Compound PP (1.4 g, 1.5 mmol), and DIEA (830 μl, 4.8 mmol) in DMF (10 mL) was added HATU (679 mg, 1.8 mmol). The reaction mixture was stirred at ambient temperature for 20 min. The pH of the reaction mixture was adjusted to pH 5 with AcOH at ambient temperature. The obtained solution was diluted with DMSO (20 mL) and water (20 mL), and subjected to HPLC purification. [Nanosyn-Pack YMC-ODS-A (100-10) C-18 column (75×500 mm); flow rate: 250 mL/min; injection volume 5 mL×10; mobile phase A: 100% water, 0.1% TFA; mobile phase B: 100% ACN, 0.1% TFA; isocratic elution at 0% B in 4 min, gradient elution from 0% B to 90% B in 60 min, detection at 254 nm]. Fractions containing the desired product were combined and evaporated in vacuo to afford Compound QQ in 90% yield (1.5 g, 1.3 mmol) as a white solid. LC-MS [M+H] 1094.8 (C₅₃H₇₅N₉O₁₄S+H, calc: 1095.3).

### Preparation of N-1-[(S)-2-(oxycodone-6-enol-carbonyl-methyl-amino)-2-carbonyl-sarcosine-ethyl amine]-arginine-glycine-acetate (Compound KC-7)

Compound QQ (1.5 g, 1.3 mmol) was treated with 5% m-cresol in TFA (10 mL) for 1 h. The crude product was then precipitated via addition of Et₂O (1 L). The precipitate was washed with Et₂O (2×100 mL) and dried in vacuo. The resultant solid was dissolved in water (50 mL) and subjected to HPLC purification. [Nanosyn-Pack Microsorb (100-10) C-18 column (50×300 mm); flow rate: 100 mL/min; injection volume 15 mL; mobile phase A: 100% water, 0.1% TFA; mobile phase B: 100% ACN, 0.1% TFA; gradient elution from 0% to 10% B in 15 min, isocratic elution at 10% B in 20 min, gradient elution from 10% B to 40% B in 60 min; detection at 254 nm]. Fractions containing the desired product were combined and concentrated in vacuo. The residue was dissolved in 0.1 N HCl (˜50 mL) and lyophilized overnight to provide the hydrochloric salt of Compound KC-7 in 75% yield (867 mg, 1.0 mmol, 98% purity) as a foamy solid. LC-MS [M+H] 786.4 (C₃₆H₅₁N₉O₁₁+H, calc: 786.9).

### Example 13

### Synthesis of N-1-[3-(oxycodone-6-enol-carbonyl-methyl-amino)-2,2-dimethyl-propylamine] (Compound KC-22) and N-1-[3-(oxycodone-6-enol-carbonyl-methyl-amino)-2,2-dimethyl-propylamine]-arginine-glycine-malonate (Compound KC-8)

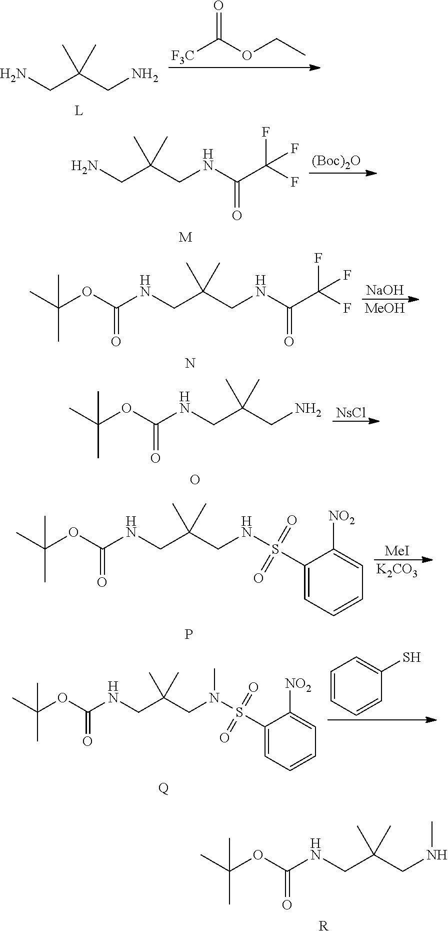

### Preparation of Compound M

A solution of 2,2-dimethyl-1,3-diamino propane (Compound L) (48.0 g, 470.6 mmol) in THF (1.0 L) was cooled in an ice bath. Ethyl trifluoroacetate (56 mL, 471 mmol) was added over 30 min via syringe. The mixture was allowed to warm up to ambient temperature, and stirring was continued for 14 h. The mixture was then concentrated in vacuo to half of its original volume to give crude Compound M as a THF solution, which was used without further purification in the next reaction. LC-MS [M+H] 199.6 (C₇H₁₃F₃N₂O+H, calc: 199.1).

### Preparation of Compound N

To a crude solution of Compound M (from previous step) in THF (500 mL) and chilled in an ice bath was added (Boc)₂O in small portions over 15 min. The mixture was stirred at ambient temperature for 15 h. The reaction was then concentrated in vacuo to give intermediate Compound N in 84% yield (over two steps) (120.0 g, 402.4 mmol) as a sticky oil. LC-MS [M+H] 299.2 (C₁₂H₂₁F₃N₂O₃+H, calc: 299.2). Compound N was used directly in the next reaction without further purification.

### Preparation of Compound O

Compound N (120 g, 403 mmol) was dissolved in CH₃OH (500 mL) and stirred at ambient temperature. NaOH (100 mL, 10 N aq.) was added dropwise. The mixture was then stirred in a pre-heated oil bath at 50° C. for 3 h. The mixture was cooled to ambient temperature and diluted with water (500 mL). Solvents were then removed in vacuo. The residue was extracted with CHCl₃ (3×100 mL). The combined CHCl₃ solution was dried over Na₂SO₄, filtered and concentrated in vacuo to afford crude Compound O in 95% yield (77.0 g, 381 mmol). LC-MS [M+H] 203.8 (C₁₀H₂₂N₂O₂+H, calc: 203.2). Compound O was used directly in the next reaction without further purification.

### Preparation of Compound P

Compound O (97.0 g, 480 mmol) was dissolved in CH₂Cl₂ (750 mL). To this was added K₂CO₃ (75.0 g, 542.6 mmol) in one portion, followed by portion wise addition of 2-nosyl chloride (108.0 g, 487.3 mmol). The reaction mixture was stirred at ambient temperature for 15 h. Water (200 mL) was then added, and the layers were separated. The aqueous layer was again extracted with CH₂Cl₂. The combined CH₂Cl₂ solution was dried over Na₂SO₄, filtered and concentrated in vacuo. The residue was purified by silica gel chromatography using 3/1 Hexanes/EtOAc to give intermediate Compound P in 83% yield (155.0 g, 400.5 mmol) as a white solid. LC-MS [M+H] 388.8 (C₁₆H₂₅N₃O₆S+H, calc: 388.1).

### Preparation of Compound R

Compound P (155.0 g, 400.5 mmol) was dissolved in DMF (500 mL) at ambient temperature. K₂CO₃ (83.0 g, 600 mmol) was added in one portion. The mixture was then cooled in an ice water bath. MeI (37.0 mL, 593 mmol) was added in small portions via syringe over 10 min. The mixture was then warmed up to ambient temperature, and stirring was continued at this temperature for another 2 h. The mixture was concentrated in vacuo until ˜50 mL remained. The remaining mixture containing intermediate Compound Q was cooled in an ice water bath. While stirring, thiophenol (100 mL, 978 mmol) was added via a syringe. The resulted mixture was stirred at ambient temperature for 6 h. Water (500 mL) was added. The mixture was extracted with EtOAc (100 mL, then 2×500 mL). The combined EtOAc extracts were extracted with 2N HCl (400 mL, then 2×200 mL). The HCl extracts were pooled and washed with DCM (500 mL). The acidic solution was then chilled in an ice water bath and basified by adding 10 N NaOH until pH ˜13. CHCl₃ (400 mL, then 2×200 mL) was then used to extract the aqueous solution. The combined CHCl₃ solution was dried over Na₂SO₄ and filtered. Evaporation of solvents in vacuo afforded Compound R in 67% yield (58.0 g, 268.5 mmol) as a slightly yellowish oil. LC-MS [M+H] 217.6 (C₁₁H₂₄N₂O₂+H, calc: 217.2).

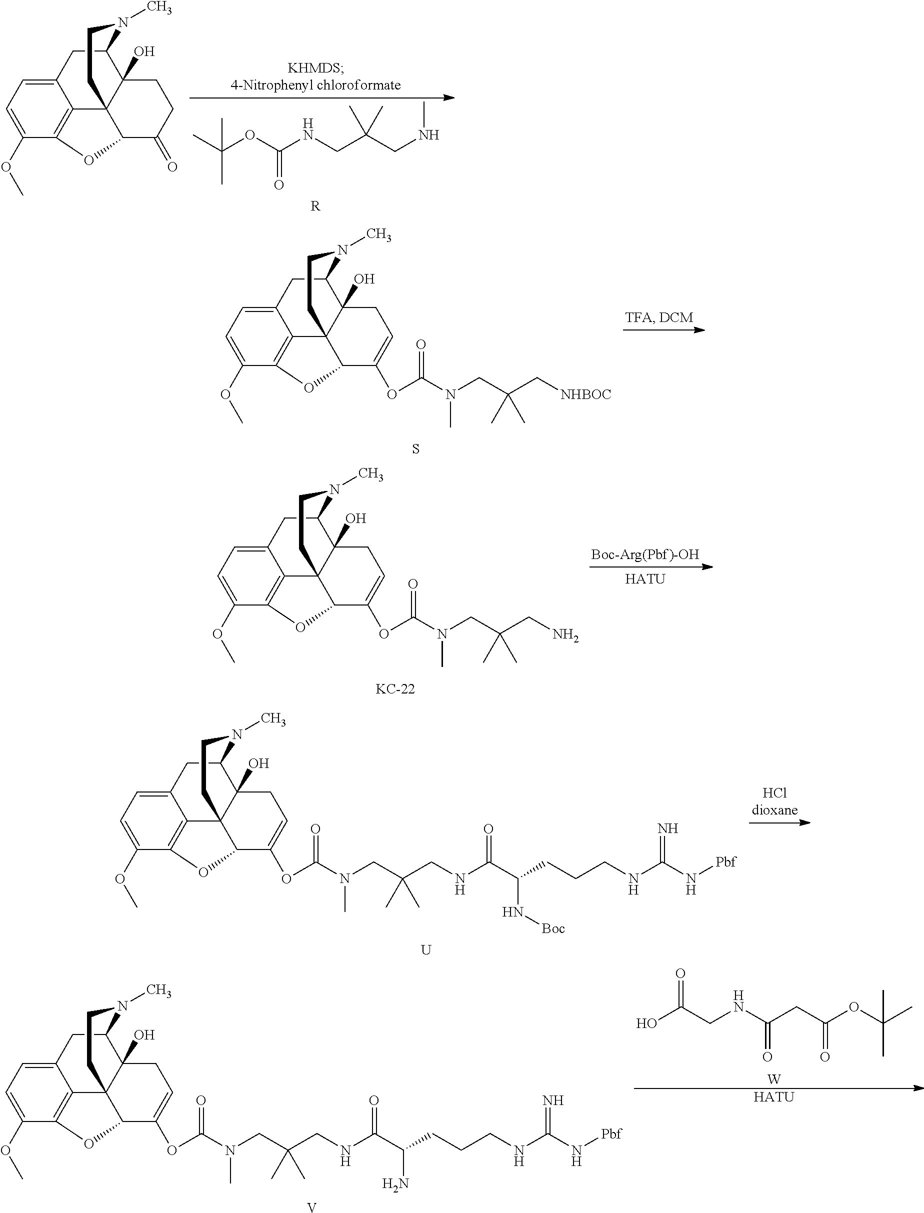

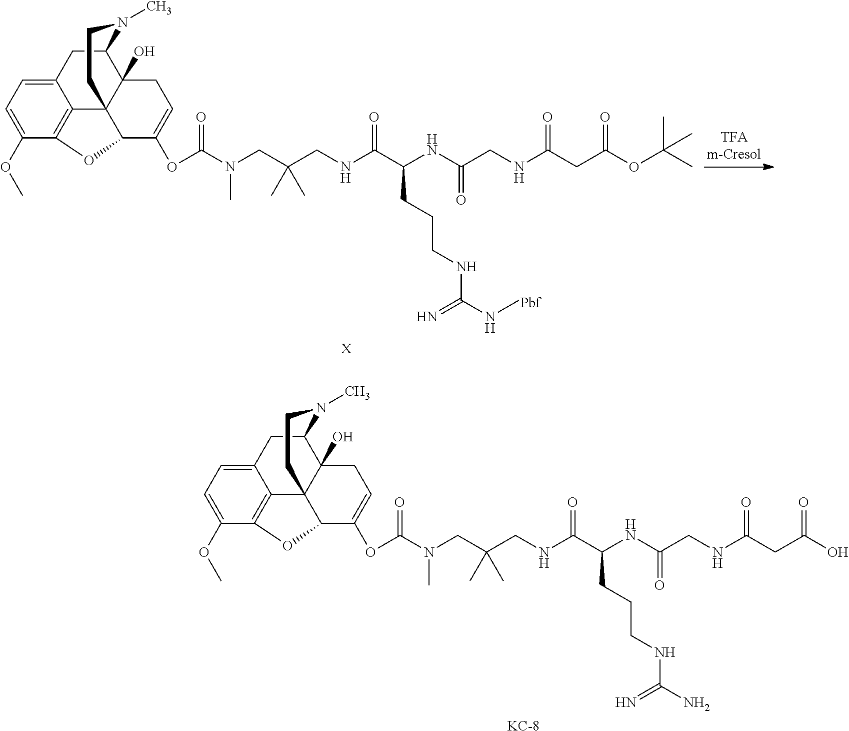

### Preparation of Compound S

Oxycodone free base (10.0 g, 31.75 mmol) was dissolved in dry THF (150 mL) and the mixture was cooled to −70° C. using a dry ice/acetone bath. KHMDS (64.0 mL, 128.0 mmol, 0.5 M in toluene) was added via syringe over 15 min. The mixture was stirred under N₂ for an additional 30 min (bath temperature −70° C.). In a separate flask was added 4-nitrophenyl chloroformate (6.4 g, 31.75 mmol) and THF (10 mL). This mixture was also chilled to −70° C. using a dry ice/acetone bath. The mixture in the first flask (containing deprotonated oxycodone) was then transferred via cannula to the second flask (containing 4-nitrophenyl chloroformate). The transfer occurred over ˜30 min, with the temperature of both flasks being maintained at −70° C. during the course of the transfer. The resulted reaction mixture was further stirred at −70° C. for 30 min. A solution of Compound R (6.9 g, 31.94 mmol) in THF (15 mL) was then added via syringe. The mixture was allowed to stir at −70° C. for 30 min, and then concentrated in vacuo to afford a gel like residue (˜90% solvent removal). The residue was let stand at ambient temperature for 15 h. It was then taken into EtOAc (200 mL) and washed with sat. aq. NaHCO₃ (5×50 mL), water (3×40 mL) and brine (50 mL). The residue from the concentrated EtOAc layer was then purified by silica gel chromatography, using 10/1 CH₃C1/MeOH to give Compound S in 62% yield (11.0 g, 19.7 mmol). LC-MS [M+H] 559.1 (C₃₀H₄₃N₃O₇+H, calc: 558.3).

### Preparation of N-1-[3-(oxycodone-6-enol-carbonyl-methyl-amino)-2,2-dimethyl-propylamine](Compound KC-22)

A solution of Compound S (11.0 g, 19.7 mmol) was treated with a mixture of TFA and DCM (30 mL/30 mL) for 2 h at ambient temperature. Solvents were then removed in vacuo until a volume of ˜5 mL remained. Et₂O (250 mL) was added to precipitate out the product. The resulting precipitate was filtered, washed with Et₂O (50 mL) and dried to afford crude Compound KC-22 in 97% yield (11.0 g, 19.2 mmol, 90% purity) as a white solid. LC-MS [M+H] 458.9 (C₂₅H₃₅N₃O₅+H, calc: 458.3). Compound KC-22 was used directly in the next reaction without further purification.

### Preparation of Compound U

A solution of Boc-Arg(Pbf)-OH (9.4 g, 17.8 mmol), Compound KC-22 (11.0 g, 19.7 mmol, 90% pure) and NEt₃ (10.0 mL, 71.7 mmol) in DMF (80 mL) was cooled in an ice bath, followed by the addition of HATU (6.8 g, 17.9 mmol) in portions over 10 min. The ice bath was then removed, and the reaction mixture was stirred at ambient temperature for an additional 1 h. The mixture was diluted with EtOAc (150 mL) and extracted with water (3×50 mL) and brine (50 mL). The organic layer was dried over Na₂SO₄ and filtered; removal of solvents in vacuo provided crude Compound U. Compound U was purified by flash chromatography using CH₂Cl₂ and MeOH to afford Compound U in 79% yield (13.7 g, 14.2 mmol) as a foamy solid. LC-MS [M+H] 967.5 (C₄₉H₇₁N₇O₁₁S+H, calc: 966.5).

### Preparation of Compound V

A solution of Compound U (13.7 g, 14.2 mmol) was treated with HCl (4.0M solution in 1,4-dioxane, 40 mL) at ambient temperature for 90 min. Solvents were removed in vacuo, and the residue was treated with Et₂O (100 mL). The resulting precipitate was filtered off, washed with Et₂O (2×25 mL), and dried to afford crude Compound V in 91% yield (12.1 g, 12.9 mmol) as a white solid. LC-MS [M+H] 867.8 (C₄₄H₆₃N₇O₉S+H, calc: 866.4). Compound V was used directly in the next reaction without further purification.

### Preparation of Compound X

To a solution of Compound V (73.3 g, 78.14 mmol, as HCl salt), N-carboxymethyl-malonate tert-butyl ester (Compound W) (17.0 g, 78.34 mmol), and NEt₃ (33.0 mL, 236.7 mmol) in DMF (500 mL) at 0° C. was added HATU (30.6 g, 80.47 mmol) in portions over 10 min. The reaction mixture was stirred at ambient temperature for 1 h. Water (500 mL) was added and the mixture was extracted with EtOAc (750 mL). The EtOAc extracts were washed with water (2×250 mL), NaHCO₃ (2×200 mL) and brine (250 mL). The organic layer was dried over Na₂SO₄ and filtered. The solution was concentrated, and the residue was purified by a silica gel column, using gradient 1-10% MeOH in CH₂Cl₂, to provide Compound X in 43% yield (36.0 g, 33.8 mmol) as a white solid. LC-MS [M+H] 1067.2 (C₅₃H₇₆N₈O₁₃S+H, calc: 1065.5).

### Preparation of N-1-[3-(oxycodone-6-enol-carbonyl-methyl-amino)-2,2-dimethyl-propylamine]-arginine-glycine-malonate (Compound KC-8)

Compound X (36.0 g, 33.8 mmol) was treated with a mixture of TFA (60 mL) and m-cresol (2.0 mL) at ambient temperature. The reaction progress was monitored by LC/MS. After 4 h, the mixture was concentrated in vacuo to remove most of the volatiles (˜0.90% solvent removed). The residue was treated with ethyl ether (1 L), and a white precipitate was formed. The clear supernatant was removed and the precipitate was washed with ethyl ether (1 L). The solid was then concentrated and subjected to HPLC purification. [Nanosyn-Pack Microsorb (100-10) C-18 column (50×300 mm); flow rate: 100 mL/min; injection volume 15 mL; mobile phase A: 100% water, 0.1% TFA; mobile phase B: 100% ACN, 0.1% TFA; gradient elution from 0% to 20% B in 30 min, isocratic elution at 20% B in 30 min, gradient elution from 20% B to 45% B in 35 min; detection at 254 nm]. Fractions containing the desired compound were combined and concentrated in vacuo. The residue was dissolved in ACN (60 mL) and 0.1 N HCl (200 mL), and lyophilized to provide Compound KC-8 in 69.6% yield (19.5 g, 23.5 mmol, 99.4% purity) as a white foam. LC-MS [M+H] 758.5 (C₃₆H₅₂N₈O₁₀+H, calc: 757.4).

### Biological Data of Ketone-modified Opioid Prodrugs

### Example 14

### Oral Administration of Compound KC-7 Co-Dosed with Trypsin Inhibitor Compound 109 to Rats

This Example demonstrates the ability of a trypsin inhibitor to affect the ability of Compound KC-7 to release oxycodone into plasma when Compound KC-7 is administered orally to rats.

Saline solutions of prodrug Compound KC-7 (which can be prepared as described in the examples herein) were dosed at 5 mg/kg (5.8 μmol/kg) or 50 mg/kg (58 μmol/kg). The rats were co-dosed with increasing concentrations of Compound 109 (Catalog No. 3081, Tocris Bioscience, Ellisville, Mo., USA or Catalog No. WS38665, Waterstone Technology, Carmel, Ind., USA) as indicated in Table 6 via oral gavage into jugular vein-cannulated male Sprague Dawley rats (4 per group) that had been fasted for 16-18 h prior to oral dosing. At specified time points, blood samples were drawn, harvested for plasma via centrifugation at 5,400 rpm at 4° C. for 5 min, and 100 microliters (μl) plasma transferred from each sample into a fresh tube containing 2 μl of 50% formic acid. The tubes were vortexed for 5-10 seconds, immediately placed in dry ice, and then stored in a −80° C. freezer until analysis by HPLC/MS.

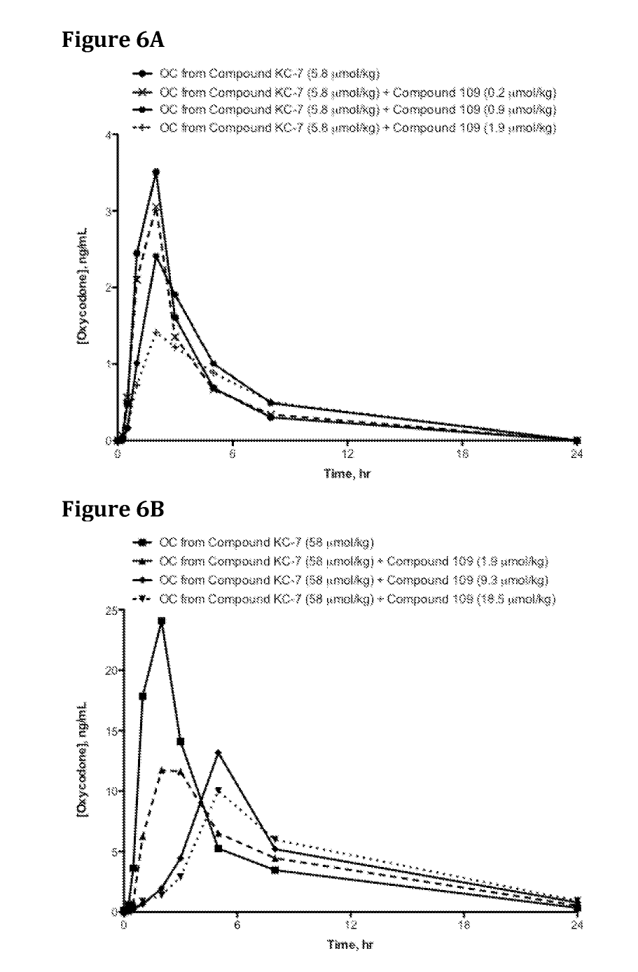

Table 6, FIG. 6A and FIG. 6B provide oxycodone exposure results for rats administered with different doses of Compound KC-7, each co-dosed with increasing amounts of trypsin inhibitor Compound 109. Results in Table 4 are reported, for each group of four rats, as (a) maximum plasma concentration (Cmax) of oxycodone (OC) (average±standard deviation), (b) time after administration of Compound KC-8 to reach maximum oxycodone concentration (Tmax) (average±standard deviation) and (c) area under the curve (AUC) from 0 to 24 h (average±standard deviation).

TABLE 6
Cmax, Tmax and AUC values of oxycodone in rat plasma

| KC-7 | KC-7 | Compound | Compound | | | |
|---|---|---|---|---|---|---|
| Dose, | Dose, | 109 Dose, | 109 Dose, | OC Cmax ± | | AUC ± sd, |
| mg/kg | μmol/kg | mg/kg | μmol/kg | sd, ng/mL | Tmax ± sd, h | ng * h/mL |
| 5 | 5.8 | 0 | 0* | 3.73 ± 0.91 | 1.75 ± 0.50 | 10.1 ± 3.8 |
| 5 | 5.8 | 0.1 | 0.2* | 3.05 ± 1.4 | 2.00 ± 0.0 | 9.08 ± 3.3 |
| 5 | 5.8 | 0.5 | 0.9* | 2.55 ± 0.57 | 2.25 ± 0.50 | 9.35 ± 1.1 |
| 5 | 5.8 | 1 | 1.9* | 1.46 ± 0.45 | 2.25 ± 0.50 | 6.83 ± 2.0 |
| 50 | 58 | 0 | 0\hat{ } | 24.7 ± 4.8 | 1.75 ± 0.50 | 109 ± 15 |
| 50 | 58 | 1 | 1.9\hat{ } | 13.5 ± 3.3 | 2.25 ± 0.50 | 97.2 ± 16 |
| 50 | 58 | 5 | 9.3\hat{ } | 13.2 ± 3.4 | 5.00 ± 0.0 | 80.7 ± 24 |
| 50 | 58 | 10 | 18.5\hat{ } | 10.0 ± 1.4 | 5.00 ± 0.0 | 95.8 ± 16 |

*Lower limit of quantitation was 0.100 ng/ml
\hat{ }Lower limit of quantitation was 0.0500 ng/ml

FIG. 6A compares mean plasma concentrations over time of oxycodone release following PO administration of 5 mg/kg (5.8 μmol/kg) of prodrug Compound KC-7 with increasing amounts of co-dosed trypsin inhibitor Compound 109 to rats.

FIG. 6B compares mean plasma concentrations over time of oxycodone release following PO administration of 50 mg/kg (58 μmol/kg) of prodrug Compound KC-7 with increasing amounts of co-dosed trypsin inhibitor Compound 109 to rats.

The results in Table 6, FIG. 6A and FIG. 6B indicate Compound 109's ability to attenuate Compound KC-7's ability to release oxycodone in rats in a dose-dependent manner, as indicated by suppressed Cmaxand/or delayed Tmax.

### Example 15

### Oral Administration of a Single Dose Unit and of Multiple Dose Units of a Composition Comprising Prodrug Compound KC-8 and Trypsin Inhibitor Compound 109 in Rats

This Example demonstrates the effect of oral administration of single and multiple dose units comprising prodrug Compound KC-8 and trypsin inhibitor Compound 109 to rats.

Saline solutions of Compound KC-8 (which can be prepared as described in the examples herein) were dosed orally to rats (4 rats per group) at increasing concentrations ranging from 5 to 50 mg/kg (from 6 to 60 μmol/kg), wherein a single dose was represented as 5 mg/kg (6 μmol/kg) Compound KC-8 in the absence of trypsin inhibitor.

A second set of rats (4 rats per group) were co-dosed orally with prodrug Compound KC-8 and trypsin inhibitor Compound 109 (Catalog No. 3081, Tocris Bioscience, or Catalog No. WS38665, Waterstone Technology) as described below and indicated in Table 5. Specifically, a saline solution of a composition comprising 5 mg/kg (6 μmol/kg) Compound KC-8 and 0.5 mg/kg (1 mmol/kg) Compound 109, representative of a single dose unit, was administered via oral gavage to a group of 4 rats. It is to be noted that the mole-to-mole ratio of trypsin inhibitor-to-prodrug (109-to-KC-8) is 0.17-to-1; as such this dose unit is referred to herein as a 109-to-KC-8 (0.17-to-1) dose unit. Saline solutions representative of 2 dose units, 3 dose units, 4 dose units, 6 dose units, 8 dose units, and 10 dose units (i.e., as indicated in Table 5) of the 109-to-KC-8 (0.17-to 1) dose unit were similarly administered to additional groups of 4 rats.

All rats were jugular vein-cannulated male Sprague Dawley rats that had been fasted for 16-18 h prior to oral dosing. At specified time points, blood samples were drawn, harvested for plasma via centrifugation at 5,400 rpm at 4° C. for 5 min, and 100 microliters (μl) plasma transferred from each sample into a fresh tube containing 2 μl of 50% formic acid. The tubes were vortexed for 5-10 seconds, immediately placed in dry ice, and then stored in a −80° C. freezer until analysis by HPLC/MS.

Results in Table 7 are reported, for each group of rats, as (a) maximum plasma concentration (Cmax) of oxycodone (OC) (average±standard deviation), (b) time after administration of Compound KC-8 to reach maximum oxycodone concentration (Tmax) (average±standard deviation) and (c) area under the curve (AUC) from 0 to 24 h (average±standard deviation).

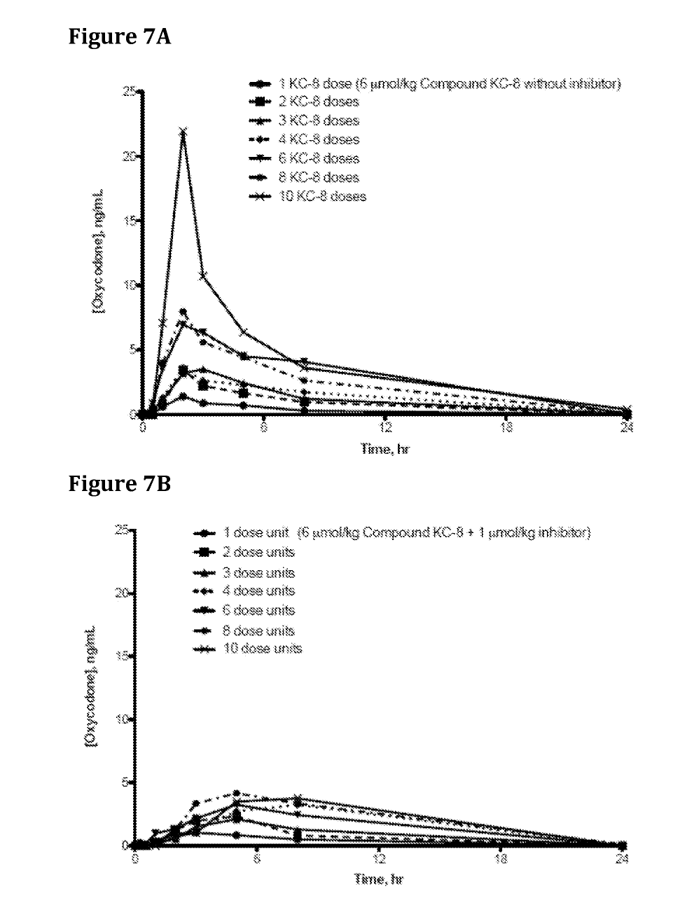

Table 7 (top half) and FIG. 7A provide oxycodone exposure results in plasma for rats administered 1, 2, 3, 4, 6, 8 and 10 doses of Compound KC-8 in the absence of trypsin inhibitor. Table 7 (bottom half) and FIG. 7B provide oxycodone exposure results in plasma for rats administered 1, 2, 3, 4, 6, 8 and 10 dose units of the 109-to-KC-8 (0.17-to-1) dose unit. Oxycodone Cmax, Tmax and AUC values are reported, for each group of rats, as (a) maximum plasma concentration (Cmax) of oxycodone (OC) (average±standard deviation), (b) time after administration of Compound KC-8 to reach maximum oxycodone concentration (Tmax) (average±standard deviation) and (c) area under the curve (AUC) from 0 to 24 h (average±standard deviation).

TABLE 7
Cmax, Tmax and AUC values of oxycodone in rat plasma

| | KC-8 | KC-8 | 109 | 109 | OC Cmax ± | | |
|---|---|---|---|---|---|---|---|
| Amount | Dose, | Dose, | Dose, | Dose, | sd, | | AUC ± sd, |
| (Multiple) | mg/kg | μmol/kg | mg/kg | μmol/kg | ng/mL | Tmax ± sd, h | ng * h/mL |
| 1 KC-8 dose | 5 | 6 | 0 | 0* | 1.39 ± 0.84 | 2.00 ± 0.0 | 5.34 ± 1.9 |
| 2 KC-8 doses | 10 | 12 | 0 | 0^{#} | 3.47 ± 1.6 | 2.25 ± 0.50 | 13.9 ± 3.2 |
| 3 KC-8 doses | 15 | 18 | 0 | 0\hat{ } | 4.23 ± 2.2 | 2.50 ± 0.58 | 24.3 ± 16 |
| 4 KC-8 doses | 20 | 24 | 0 | 0\hat{ } | 3.68 ± 1.7 | 2.25 ± 0.50 | 25.2 ± 14 |
| 6 KC-8 doses | 30 | 36 | 0 | 0\hat{ } | 7.42 ± 1.7 | 3.50 ± 3.0 | 65.1 ± 25 |
| 8 KC-8 doses | 40 | 48 | 0 | 0\hat{ } | 9.16 ± 5.3 | 2.25 ± 0.50 | 45.1 ± 26 |
| 10 KC-8 doses | 50 | 60 | 0 | 0^{#} | 21.9 ± 5.3 | 2.00 ± 0.0 | 83.8 ± 24 |
| 1 dose unit | 5 | 6 | 0.5 | 1* | 1.09 ± 0.55 | 3.25 ± 1.3 | 5.66 ± 2.0 |
| 2 dose units | 10 | 12 | 1 | 2\hat{ } | 2.82 ± 0.97 | 3.50 ± 1.0 | 11.6 ± 1.5 |
| 3 dose units | 15 | 18 | 1.5 | 3\hat{ } | 2.13 ± 0.75 | 4.50 ± 1.0 | 10.3 ± 4.3 |
| 4 dose units | 20 | 24 | 2 | 4\hat{ } | 3.34 ± 2.1 | 7.25 ± 1.5 | 14.6 ± 9.1 |
| 6 dose units | 30 | 36 | 3 | 6\hat{ } | 3.27 ± 1.2 | 5.00 ± 0.0 | 28.6 ± 19 |
| 8 dose units | 40 | 48 | 4 | 7\hat{ } | 4.63 ± 3.3 | 4.50 ± 1.0 | 45.2 ± 39 |
| 10 dose units | 50 | 60 | 5 | 9^{#} | 4.24 ± 0.71 | 6.50 ± 1.7 | 20.0 ± 3.5 |

*Lower limit of quantitation was 0.100 ng/mL
\hat{ }Lower limit of quantitation was 0.0500 ng/mL
^{#}Lower limit of quantitation was 0.500 ng/mL

FIG. 7A compares mean plasma concentrations over time of oxycodone release following PO administration of a single dose and of multiple doses of Compound KC-8 dosed in the absence of trypsin inhibitor.

FIG. 7B compares mean plasma concentrations over time of oxycodone release following PO administration of a single dose unit and of multiple dose units of a composition comprising prodrug Compound KC-8 and trypsin inhibitor Compound 109.

The results in Table 7, FIG. 7A and FIG. 7B indicate that administration of multiple dose units (as exemplified by 1, 2, 3, 4, 6, 8 and 10 dose units of the 109-to-KC-8 (0.17-to 1) dose unit) results in a plasma oxycodone concentration-time PK profile that is not dose proportional to the plasma oxycodone concentration-time PK profile of the single dose unit. In addition, the PK profile of the multiple dose units (e.g., FIG. 7B) was modified compared to the PK profile of the equivalent dosage of prodrug in the absence of trypsin inhibitor (e.g., FIG. 7A).

### Synthesis of Ketone-modified Opioid Prodrugs with Heterocyclic Linkers

### Example 16

### Synthesis of N-(oxycodone-6-enol-carbonyl)piperidine-2-methylamine (Compound KC-11) and N-(oxycodone-6-enol-carbonyl)piperidine-2-methylamine-L-arginine-L-alanine-acetate (Compound KC-13)

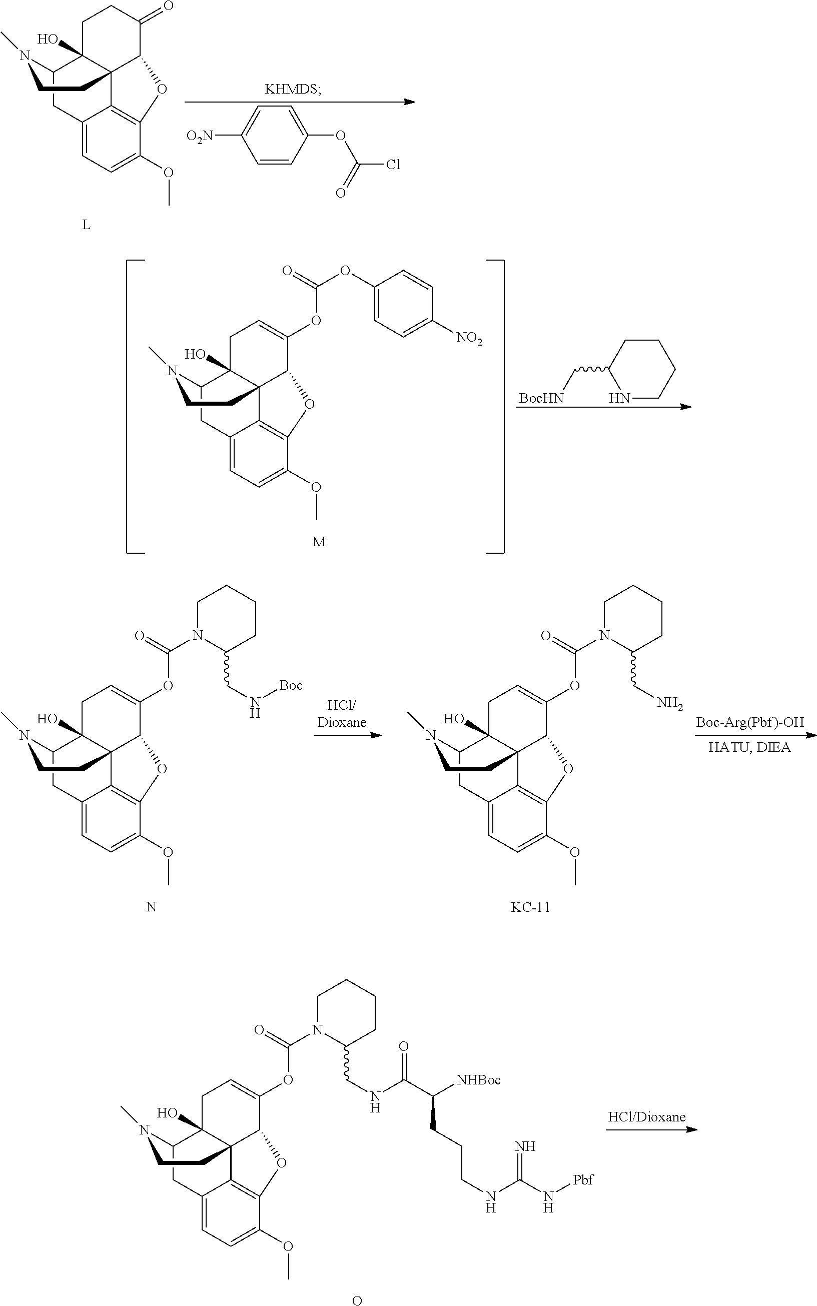

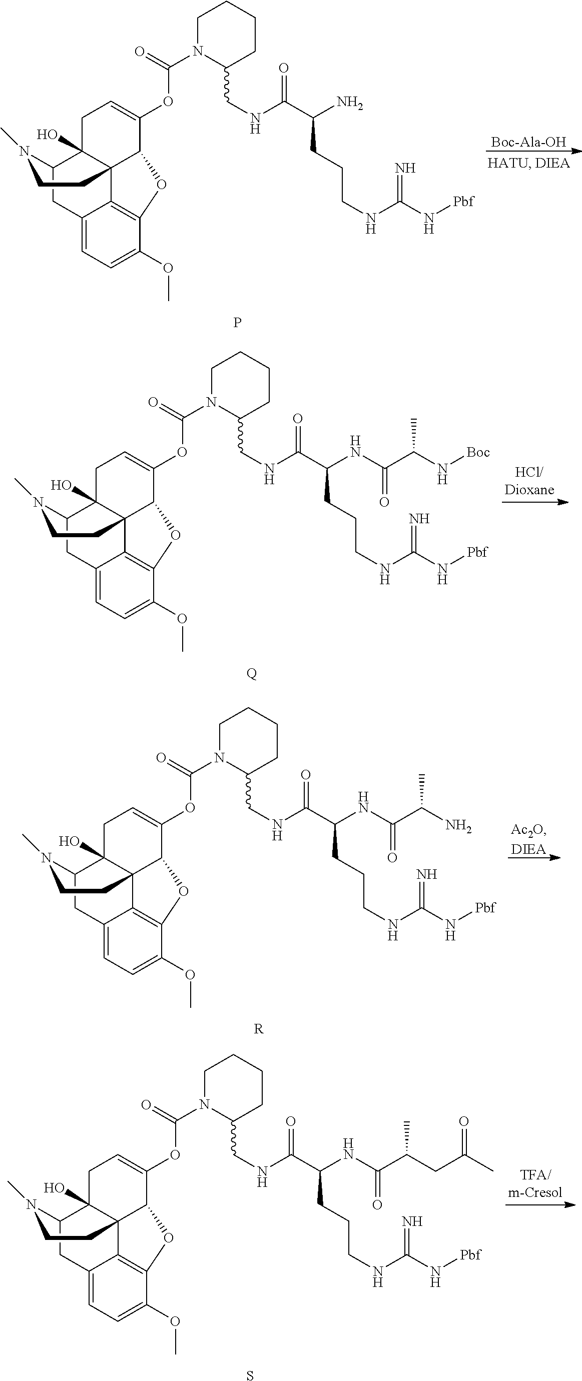

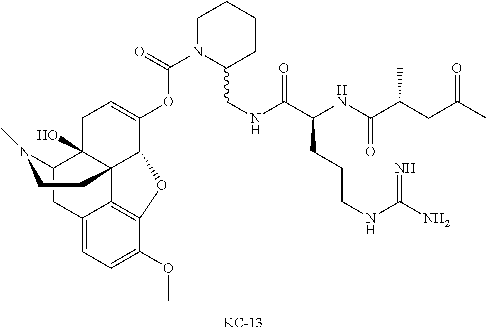

### Preparation of Oxycodone Free Base (L)

Oxycodone hydrochloride (21.0 g, 59.7 mmol) was dissolved in water (250 mL). This solution was basified with saturated aqueous NaHCO₃ (to pH 8-9) and extracted with DCM (3×250 mL). The combined organic layer was dried over Na₂SO₄ and filtered; removal of solvents in vacuo afforded Compound L in 98% yield (18.5 g, 58.8 mmol) as a white solid. LC-MS [M+H] 316.1 (C₁₈H₂₁NO₄⁺H, calc: 316.2). Compound L was used directly in the next reaction without further purification.

### Preparation of Compound N

To a solution of Compound L (14.71 g, 46.7 mmol) in THF (250 mL) at −60° C. was added 0.5 M KHMDS solution in THF (103 mL) dropwise. After stirring at −60° C. for 30 min, the reaction mixture was added to a solution of 4-nitrophenyl chloroformate at −60° C. (9.41 g, 46.7 mmol) in THF (200 mL). This reaction mixture was then stirred for 30 min at −60° C., followed by addition of piperidine-2-yl-methylcarbamic acid tert-butyl ester, also referred to herein as (R,S)-piperidine-2-yl-methylcarbamic acid tert-butyl ester, (5.0 g, 23.3 mmol) in portions. The reaction was allowed to warm to ambient temperature and then stirred for 18 h. The reaction was then concentrated in vacuo, and the residue diluted with EtOAc (500 mL). The mixture was then washed with water (2×250 mL) and brine (250 mL). The organic layer was separated, dried over Na₂SO₄, and filtered. Removal of solvents in vacuo afforded crude Compound N. Crude Compound N was purified by flash chromatography using 100% EtOAc. Removal of solvent in vacuo afforded Compound N in 50% yield (6.5 g, 11.7 mmol) as a white solid. LC-MS [M+H] 556.1 (C₃₀H₄₁N₃O₇+H, calc: 555.3).

### Preparation of N-(oxycodone-6-enol-carbonyl)piperidine-2-methylamine) (KC-11)

A solution of Compound N (6.5 g, 11.7 mmol) in 1,4-dioxane (100 mL) was treated with hydrogen chloride (4.0M solution in 1,4-dioxane, 100 mL). After 1 h, most of the 1,4-dioxane was removed in vacuo to −20 mL remaining. To this solution was added Et₂O (˜750 mL). The product was then precipitated as an HCl salt. The precipitate was filtered, washed with ether and dried in vacuo to afford Compound KC-11 in 97% yield (5.96 g, 11.3 mmol) as a white solid. LC-MS [M+H] 456.3 (C₂₅H₃₃N₃O₅+H, calc: 456.2). Compound KC-11 was used directly in the next reaction without further purification.

### Preparation of Compound O

To a solution of Boc-Arg(Pbf)-OH (5.94 g, 11.3 mmol), Compound KC-11 (5.95 g, 11.3 mmol) and DIEA (8.24 mL, 47.4 mmol) in DMF (100 mL) at −0° C. was added HATU (4.28 g, 11.3 mmol) in portions over 10 min. The temperature of the reaction mixture was raised to ambient temperature and stirring was continued for an additional 1 h. DMF was removed in vacuo, and the reaction mixture was diluted with EtOAc (300 mL), washed with water (3×150 mL) and brine (150 mL). The organic layer was separated, dried over Na₂SO₄, and filtered. Removal of solvents in vacuo afforded crude Compound O. This compound was purified by silica gel chromatography using CHCl₃ and 0% to 20% MeOH. Removal of solvents in vacuo afforded Compound O in 23% yield (2.5 g, 2.6 mmol) as a foamy solid. LC-MS [M+H] 964.8 (C₄₉H₆₉N₇O₁₁S+H, calc: 964.5).

### Preparation of Compound P

A solution of Compound O (2.5 g, 2.6 mmol) in 1,4-dioxane (50 mL) was treated with hydrogen chloride (4.0 M solution in 1,4-dioxane, 50 mL). After 1 h, most of the 1,4-dioxane was removed in vacuo to ˜10 mL remaining. To this solution was added Et₂O (˜500 mL). The product precipitated as an HCl salt. Precipitate was filtered off, washed with ether, and dried in vacuo to afford Compound P in 52% yield (1.25 g, 1.33 mmol) as a white solid. LC-MS [M+H] 864.6 (C₄₄H₆₁N₇O₉S+H, calc: 863.4). Compound P was used directly in the next reaction without further purification.

### Preparation of Compound Q

To a solution of Boc-Ala-OH (0.13 g, 0.66 mmol), Compound P (0.62 g, 0.66 mmol), and DIEA (0.48 mL, 2.77 mmol) in DMF (10 mL) at 5° C., was added HATU (0.25 g, 0.66 mmol) in portions over 5 min. The temperature of the reaction mixture was raised to ambient temperature, and stirring was continued for an additional 1 h. DMF was removed in vacuo; the reaction mixture was diluted with EtOAc (100 mL), and washed with water (3×50 mL) and brine (50 mL). The organic layer was separated, dried over Na₂SO₄, and filtered. Removal of solvents in vacuo afforded crude Compound Q, yield exceeded quantitative, (0.69 g, 0.66 mmol) as an off-white solid. LC-MS [M+H] 1035.6 (C₅₂H₇₄N₈O₁₂S+H, calc: 1035.5). Compound Q was used directly in the next reaction without further purification.

### Preparation of Compound R

A solution of Compound Q (0.69 g, 0.66 mmol) in 1,4-dioxane (10 mL) was treated with hydrogen chloride (4.0 M solution in 1,4-dioxane, 10 mL). After 1 h, most of the 1,4-dioxane was removed in vacuo to −2 mL remaining. To this solution was added Et₂O (˜100 mL). The product precipitated as an HCl salt. The precipitate was washed with ether and dried in vacuo to afford crude Compound R, yield exceeded quantitative, (0.67 g, 0.66 mmol) as an off-white solid. LC-MS [M+H] 935.8 (C₄₇H₆₆N₈O₁₀S+H, calc: 935.5). Compound R was used directly in the next reaction without further purification.

### Preparation of Compound S

To a solution of Compound R (0.67 g, 0.66 mmol) and DIEA (0.37 mL, 2.1 mmol) in CHCl₃ (50 mL) at ˜0° C. was added acetic anhydride (Ac₂O) (0.07 mL, 0.7 mmol); the reaction mixture was stirred at ambient temperature for 30 min. The reaction mixture was diluted with CHCl₃ (50 mL), and washed with water (2×100 mL) and brine (50 mL). The organic layer was separated, dried over Na₂SO₄, and filtered. Removal of solvents in vacuo afforded the crude Compound S, yield exceeded quantitative, (0.65 g, 0.66 mmol) as an off-white solid. LC-MS [M+H] 977.4 (C₄₉H₆₈N₈O₁₁S+H, calc: 977.5). Compound S was used directly in the next reaction without further purification.

### Preparation of N-(oxycodone-6-enol-carbonyl)piperidine-2-methylamine-L-arginine-L-alanine-acetate (KC-13)

Compound S (0.65 g, 0.66 mmol) was treated with 5% m-cresol in TFA (15 mL) for 1 h. The product was precipitated via addition of Et₂O (100 mL). The precipitate was washed with Et₂O (2×100 mL) and dried in vacuo to afford crude Compound KC-13. This product was dissolved in water (15 mL), and the solution was subjected to HPLC purification. [Nanosyn-Pack Microsorb (100-10) C-18 column (50×300 mm); flow rate: 100 mL/min; injection volume 15 mL; mobile phase A: 100% water, 0.1% TFA; mobile phase B: 100% ACN, 0.1% TFA; isocratic elution at 0% B in 5 min, gradient elution from 0% to 20% B in 20 min, isocratic elution at 20% B in 20 min, gradient elution from 20% B to 45% B in 40 min; detection at 254 nm]. Fractions containing the desired compound were combined and concentrated in vacuo. The residue was dissolved in ACN (˜2 mL) and 0.1 N HCl (˜8 mL), and lyophilized overnight to provide the hydrochloric salt of Compound KC-13 in 90% yield (0.65 g, 0.59 mmol, 93.1% purity) as a white solid. LC-MS [M+H] 725.8 (C₃₆H₅₂N₈O₈+H, calc: 725.4).

### Example 17

### Synthesis of N-(oxycodone-6-enol-carbonyl)-R-(piperidine-2-methylamine)-L-arginine-glycine-malonate (Compound KC-17)

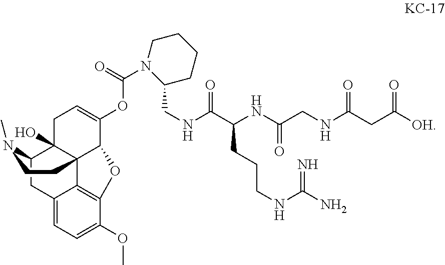

Compound KC-17 was prepared following the method described in Example 16 to prepare N-(oxycodone-6-enol-carbonyl)piperidine-2-methylamine-L-arginine-L-alanine-acetate (Compound KC-13), but using (R)-piperidine-2-yl-methylcarbamic acid tert-butyl ester instead of (R,S)-piperidine-2-yl-methylcarbamic acid tert-butyl ester, using Boc-Gly-OH instead of Boc-Ala-OH, and using mono-tert-butyl malonate instead of acetic anhydride. LC-MS [M+H] 755.5 (C₃₆H₅₀N₈O₁₀+H, calc: 755.4).

### Example 18

### Synthesis of N-(hydrocodone-6-enol-carbonyl)-R-(piperidine-2-methylamine)-L-arginine-glycine-malonate (Compound KC-31)

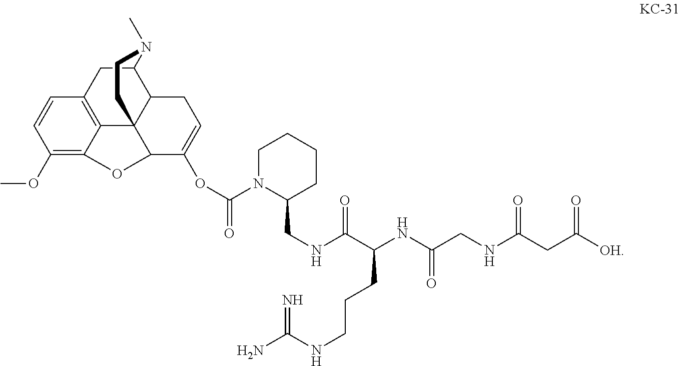

Compound KC-31 was prepared following the method described in Example 17 to prepare N-(oxycodone-6-enol-carbonyl)-R-(piperidine-2-methylamine)-L-arginine-glycine-malonate (Compound KC-17), except hydrocodone was used instead of oxycodone. LC-MS [M+H] 739.6 (C₃₆H₅₀N₈O₉+H calc:739.9).

### Example 19

### Oral Administration of a Ketone-Modified Opioid Prodrug Co-Dosed with a Trypsin Inhibitor to Rats

This Example demonstrates the ability of a trypsin inhibitor to affect the ability of a ketone-modified opioid prodrug of the embodiments to release opioid into plasma when such ketone-modified opioid prodrug was co-administered with such a trypsin inhibitor orally to rats.

Saline solutions of prodrug Compound KC-17 (which can be prepared as described in the examples herein) were co-dosed with increasing concentrations of Compound 109 (Catalog No. 3081, Tocris Bioscience, Ellisville, Mo., USA or Catalog No. WS38665, Waterstone Technology, Carmel, Ind., USA) as indicated in Table 5, to rats, via oral gavage into jugular vein-cannulated male Sprague Dawley rats (4 per group) that had been fasted for 16-18 h prior to oral dosing. At specified time points, blood samples were drawn, harvested for plasma via centrifugation at 5,400 rpm at 4° C. for 5 min, and 100 microliters (μl) plasma transferred from each sample into a fresh tube containing 2 μl of 50% formic acid. The tubes were vortexed for 5-10 seconds, immediately placed in dry ice, and then stored in a −80° C. freezer until analysis by HPLC/MS.

Table 8 provides oxycodone exposure results for rats administered a 50 mg/kg (60 μmol/kg) dose of Compound KC-17, each co-dosed with increasing amounts of trypsin inhibitor Compound 109. The oxycodone Cmax, Tmax, and AUC values in Table 8 are reported, for each group of four rats, as (a) maximum plasma concentration value (Cmax) of oxycodone (OC) (average±standard deviation), (b) time after administration of compound to reach maximum oxycodone concentration value (Tmax) (average±standard deviation) and (c) area under the curve value (AUC) from 0 to 24 h (average±standard deviation).

TABLE 8
Cmax, Tmax and AUC values of oxycodone in rat plasma

| KC- | | | | | | |
|---|---|---|---|---|---|---|
| 17 | KC-17 | Compound | Compound | | | |
| Dose, | Dose, | 109 Dose, | 109 Dose, | OC Cmax ± | | AUC ± sd, |
| mg/kg | μmol/kg | mg/kg | μmol/kg | sd, ng/mL | Tmax ± sd, h | ng * h/mL |
| 50 | 60 | 0 | 0 | 40.1 ± 10* | 1.25 ± 0.50 | 127 ± 15 |
| 50 | 60 | 1 | 1.9 | 32.4 ± 11* | 3.50 ± 3.0 | 201 ± 190 |
| 50 | 60 | 5 | 9.3 | 21.0 ± 9.0* | 4.50 ± 1.0 | 117 ± 25 |
| 50 | 60 | 10 | 18.5 | 23.2 ± 3.0* | 4.50 ± 1.0 | 145 ± 54 |

\hat{ }Lower limit of quantitation was 0.100 ng/mL
*Lower limit of quantitation was 0.500 ng/mL

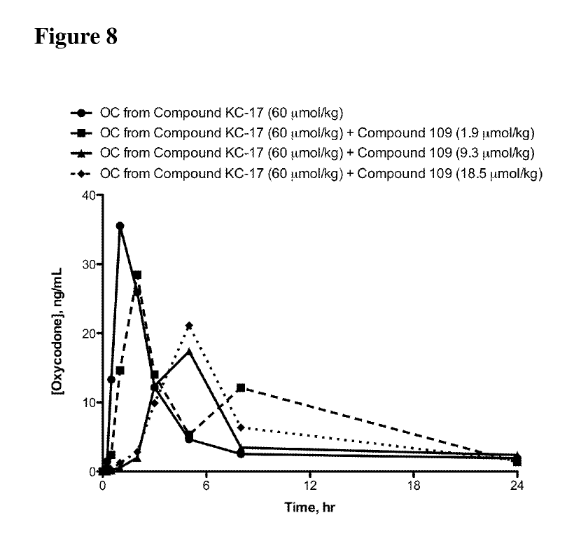

FIG. 8 compares the mean plasma concentrations over time of oxycodone release following PO administration of prodrug Compound KC-17 with increasing amounts of co-dosed trypsin inhibitor Compound 109 to rats.

The results in Table 8 and FIG. 8 indicate Compound 109's ability to attenuate release of oxycodone by prodrugs of the embodiments.

### Synthesis of Amphetamine Prodrug Compounds

### Example 20

### Synthesis of 2-Acetylamino-5-guanidino-pentanoic acid ((R)-1-methyl-2-phenyl-ethyl)-amide (Compound AM-1) and N-[4-Guanidino-1-((R)-1-methyl-2-phenyl-ethylcarbamoyl)-butyl]-malonamic acid (Compound AM-2)

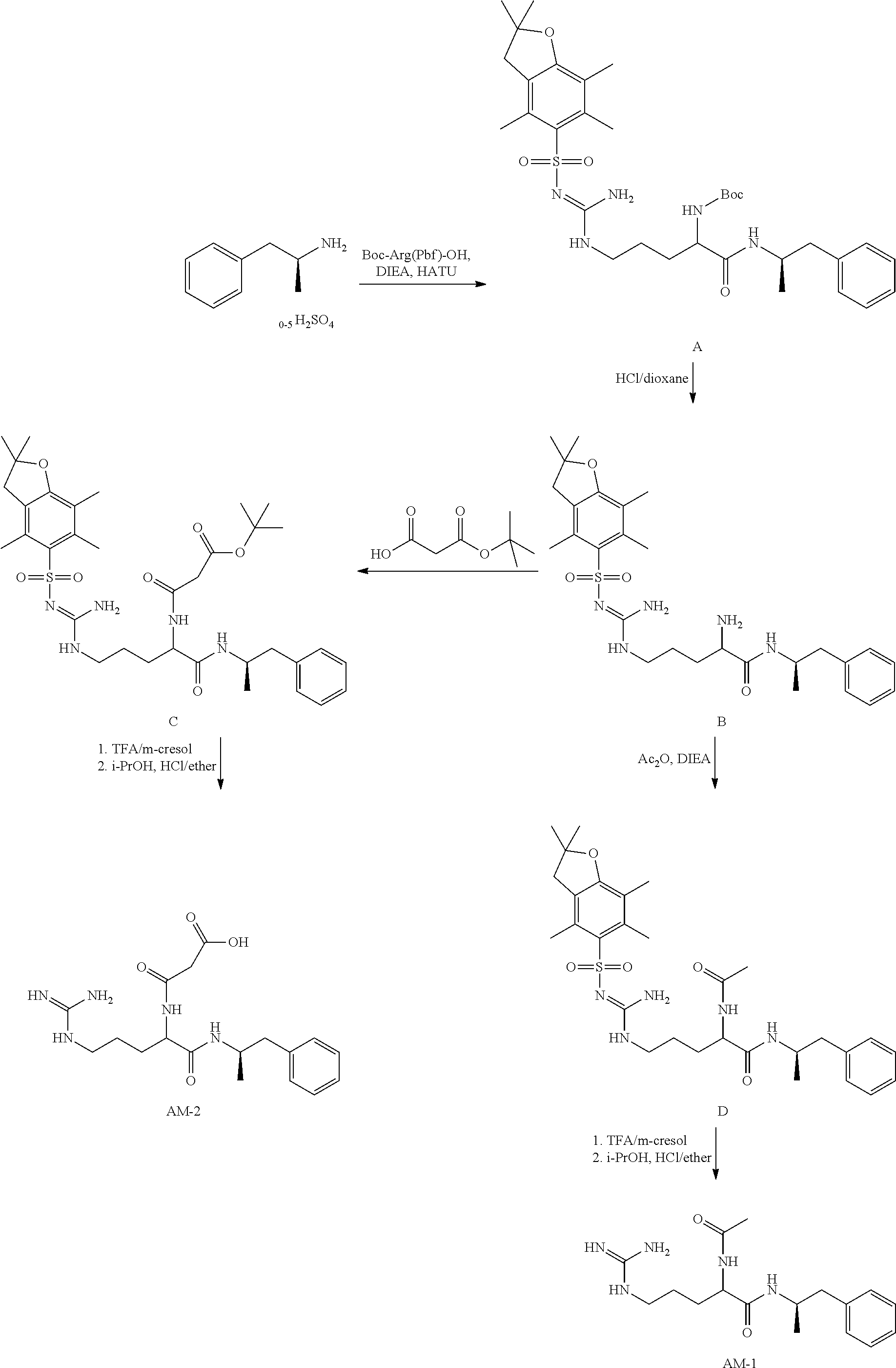

### Preparation 1

### Synthesis of [4-({Amino-[(E)-2,2,4,6,7-pentamethyl-2,3-dihydro-benzofuran-5-sulfonylimino]-methyl}-amino)-1-((R)-1-methyl-2-phenyl-ethylcarbamoyl)-butyl]-carbamic acid tert-butyl ester (A)

D-Amphetamine sulfate (5.0 g, 27.1 mmol), Boc-Arg(Pbf)-OH (10.0 g, 19.0 mmol) and HATU (10.8 g, 28.5 mmol) were suspended in DMF, brought to ˜5° C. and treated drop wise with DIEA (13.3 mL, 76 mmol) over 10 min. The reaction mixture was stirred at ˜5° C. for an additional 10 min, warmed to ambient temperature, followed by stirring for 30 min. The reaction was then diluted with EtOAc (400 mL) and poured into water (600 mL). The layers were separated, the aqueous layer extracted with EtOAc (3×300 mL) and the combined organic layers washed with 2% aq. H₂SO₄ (150 mL), water (2×600 mL) and brine (600 mL). The organic layer was dried over MgSO₄, filtered and concentrated in vacuo to give compound A (13.9 g, ˜100%) as a yellowish foamy solid. LC-MS [M+H] 644.7 (C₃₃H₄₉N₅O₆S+H, calc: 644.8). Compound A was used without further purification.

### Preparation 2

### Synthesis of 2-Amino-5-({amino-[(E)-2,2,4,6,7-pentamethyl-2,3-dihydro-benzofuran-5-sulfonylimino]-methyl}-amino)-pentanoic acid ((R)-1-methyl-2-phenyl-ethyl)-amide (B)

A solution of compound A (13.9 g, 19.0 mmol) in DCM (80 mL) was treated with 4 M HCl in dioxane (48 mL, 190 mmol) and the mixture stirred at ambient temperature for 45 min. Ether (1 L) was added and the resulting white precipitate filtered, washed with ether (50 mL), hexane (50 mL) and then dried in vacuo to give compound B as an off-white solid (11.9 g, ˜100%). LC-MS [M+H] 544.4 (C₂₈H₄₁N₅O₄S+H, calc: 544.7). Compound B was used without further purification.

### Preparation 3

### Synthesis of N-[4-({Amino-[(E)-2,2,4,6,7-pentamethyl-2,3-dihydro-benzofuran-4-sulfonylimino]-methyl}-amino-1-((R)-1-methyl-2-phenyl-ethylcarbamoyl)-butyl]-malonamic acid tert-butyl ester (C)

To a cooled solution (˜5° C.) of compound B (11.9 g, 19 mmol) and mono tert-butyl malonate (3.2 g, 20.0 mmol) in DMF (70 mL) was added portion wise, BOP (9.0 g, 20.3 mmol) over 5 min, followed by DIEA (13.3 mL, 76 mmol) drop wise over 15 min. After an additional 15 min, the ice bath was removed and the mixture warmed to ambient temperature. After 30 min, the reaction mixture was diluted with EtOAc (600 mL) and poured into water (600 mL). The layers were separated and the aqueous layer extracted with EtOAc (2×300 mL). The combined organic layers were washed with 2% aq. H₂SO₄ (150 mL), water (2×450 mL) and brine (400 mL). After drying (over MgSO₄) the solvent was evaporated in vacuo and the residue dried under high vacuum to give compound C (12.1 g, 17.6 mmol, 93%) as a yellowish foamy solid. LC-MS [M+H] 686.5 (C₃₅H₅₁N₅O₇S+H, calc: 685.9). Compound C was used without further purification.

### Preparation 4

### Synthesis of N-[4-Guanidino-1-((R)-1-methyl-2-phenyl-ethylcarbamoyl)-butyl]-malonamic acid (Compound AM-2)

A solution of compound C (12.1 g, 17.6 mmol) in 5% m-cresol/TFA (350 mL) was stirred at ambient temperature. After 45 min, the solvent was evaporated until about 100 mL volume remained, followed by dilution with hexane (500 mL). The two layers were separated, the oily precipitate (21.0 g) was concentrated and the residue dissolved in 0.1% TFA/H₂O (125 mL), sonicated for 30 min and the layers separated. The aqueous emulsion (140 mL total volume) cleared up after standing in the refrigerator overnight. This product was divided into four portions and each subjected to HPLC purification [Nanosyn-Pack Microsorb (100-10) C-18 column (50×300 mm); flow rate: 100 mL/min; injection volume: 40 mL; mobile phase A: 100% water, 0.1% TFA; mobile phase B: 100% acetonitrile, 0.1% TFA; isocratic elution at 0% B in 2 min, gradient elution to 8% B in 12 min, isocratic elution at 8% B in 30 min, gradient elution from 8% B to 33% B in 51 min; detection at UV 254 nm]. Fractions containing the desired compound were combined and concentrated in a rotary evaporator and dried under high vacuum. HPLC purifications yielded Compound AM-2 as a TFA salt (colorless viscous oil 4.0 g, 6.7 mmol, 96% purity) in 38% yield. A part of this material (640 mg, 1.06 mmol) was dissolved in i-PrOH (3 mL) and treated with 2 N HCl in ether (30 mL, 60 mmol) to give the hydrochloride salt of Compound AM-2 (430 mg, 0.95 mmol, 99% purity) in 90% yield. LC-MS [M+H] 378.3 (C₁₈H₂₇N₅O₄+H, calc: 378.4). Retention time [Chromolith SpeedRod RP-18e C18 column (4.6×50 mm); flow rate: 1.5 ml/min; mobile phase A: 0.1% TFA/water; mobile phase B 0.1% TFA/ACN; gradient elution from 5% B to 100% B over 9.6 min, detection 254 nm]: 2.15 min.

### Preparation 5

### Synthesis of 2-Acetylamino-5-({amino-[(E)-2,2,4,6,7-pentamethyl-2,3-dihydrobenzofuran-5-sulfonylimino]-methyl}-amino)-pentanoic acid ((R)-1-methyl-2-phenyl-ethyl)-amide (D)

To a solution of compound B (3.3 g, 5.0 mmol) in chloroform (30 mL) was added DIEA (2.3 mL, 13.1 mmol) and acetic anhydride (697 mg, 6.8 mmol). After stirring at ambient temperature for 30 min, the mixture was treated with 2 M EtNH₂ in THF (2.8 mL, 1.8 mmol). Stirring was continued for an additional 30 min. The solvent was then evaporated and the residue acidified to pH ˜3 with 2% aq. H₂SO₄ and extracted with EtOAc (3×100 mL). The combined organic layers were washed with water (150 mL), sat. NaHCO₃ solution (150 mL) and brine (150 mL). After drying over MgSO₄, the solvent was evaporated and the residue dried in vacuo to give compound D (2.8 g, 4.8 mmol) in 95% yield as a colorless foamy solid. LC-MS [M+H] 586.2 (C₃₀H₄₃N₅O₅S+H, calc: 586.8). Compound D was used without further purification.

### Preparation 6

### Synthesis of 2-Acetylamino-5-guanidino-pentanoic acid ((R)-1-methyl-2-phenylethyl)-amide (Compound AM-1)

A solution of compound D (2.8 g, 4.8 mmol) in 5% m-cresol/TFA (70 mL) was stirred at ambient temperature. After 45 min, TFA was evaporated and the residue taken into MeOH (10 mL), diluted with hexane (400 mL) and cooled in the refrigerator (˜4° C.) for 30 min. After separation from the hexane layer, the oily precipitate (3.8 g) was dissolved in water (30 mL) and purified by HPLC. [Nanosyn-Pack Microsorb (100-10) C-18 column (50×300 mm); flow rate: 100 mL/min; injection volume: 35 mL; mobile phase A: 100% water, 0.1% TFA; mobile phase B: 100% acetonitrile, 0.1% TFA; isocratic elution at 5% B in 5 min, gradient elution to 12% B in 7 min, isocratic elution at 12% B in 20 min, gradient elution from 12% B to 40% B in 28 min; detection at UV 254 nm]. Fractions containing the desired compound were combined and concentrated in vacuo. Traces of water were removed by dissolving the residue in i-PrOH (50 mL) followed by evaporation in vacuo (procedure was repeated twice). The residue was dissolved in i-PrOH (20 mL) and treated with 2 N HCl in ether (100 mL, 200 mmol) to give the hydrochloride salt of Compound AM-1 (1.34 g, 3.6 mmol, 99% purity) in 76% yield. LC-MS [M+H] 334.4 (C₁₇H₂₇N₅O₂+H, calc: 334.4). Retention time [Chromolith SpeedRod RP-18e C18 column (4.6×50 mm); flow rate: 1.5 ml/min; mobile phase A: 0.1% TFA/water; mobile phase B 0.1% TFA/ACN; gradient elution from 5% B to 100% B over 9.6 min, detection 254 nm]: 2.43 min.

### Example 21

### Oral Administration of Compound AM-1 and Trypsin Inhibitor Compound 109 to Rats

This Example demonstrates the ability of a trypsin inhibitor of the embodiments to affect drug release into plasma from Compound AM-1 administered orally.

Saline solutions of Compound AM-1 (which can be prepared as described in the examples herein) were dosed at 16 μmol/kg (6 mg/kg) with or without a co-dose of 55 μmol/kg (30 mg/kg) Compound 109 (Catalog No. 3081, Tocris Bioscience or Catalog No. WS38665, Waterstone Technology, Carmel, Ind.) as indicated in Table 3 via oral gavage into jugular vein-cannulated male Sprague Dawley rats (4 per group) that had been fasted for 16-18 hr prior to oral dosing. At specified time points, blood samples were drawn, harvested for plasma via centrifugation at 5,400 rpm at 4° C. for 5 min, and 100 μl plasma transferred from each sample into a fresh tube containing 2 μl of 50% formic acid. The tubes were vortexed for 5-10 seconds, immediately placed in dry ice and then stored in a −80° C. freezer until analysis by HPLC/MS.

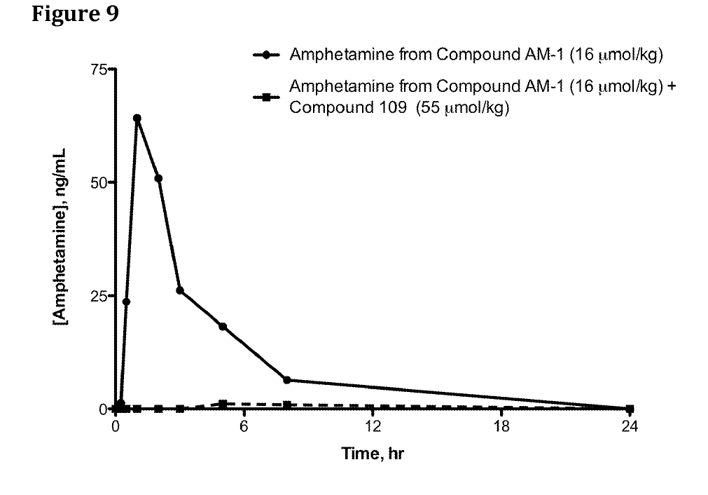

Table 9 and FIG. 9 provide amphetamine exposure results for rats administered with Compound AM-1 with or without a co-dose of trypsin inhibitor. Results in Table 9 are reported, for each group of 4 rats, as (a) maximum plasma concentration (Cmax) of amphetamine (AMP) (average±standard deviation) and (b) time after administration of Compound AM-1, to reach maximum amphetamine concentration (Tmax) (average±standard deviation).

TABLE 9
Cmax and Tmax values of amphetamine in rat plasma

| AM-1 | AM-1 | Compound | Compound | AMP | Tmax ± |
|---|---|---|---|---|---|
| Dose, | Dose, | 109 Dose, | 109 Dose, | Cmax ± sd, | sd, |
| mg/kg | μmol/kg | mg/kg | μmol/kg | ng/mL | hr |
| 6 | 16 | 0 | 0 | 64.2 ± 10 | 1.0 ± 0.0 |
| 6 | 16 | 30 | 55 | 2.08 ± 2.4 | 6.5 ± 2.1 |

Lower limit of quantitation was 1.000 ng/mL.

FIG. 9 compares mean plasma concentrations over time of amphetamine release following PO administration of Compound AM-1 with or without a co-dose of trypsin inhibitor.

The results in Table 9 and FIG. 9 indicated Compound 109's ability to attenuate Compound AM-1's ability to release amphetamine both by suppressing Cmax and by delaying Tmax.

### Example 22

### Oral Administration of Compound AM-2 and Trypsin Inhibitor Compound 109 to Rats

This Example demonstrates the ability of a trypsin inhibitor of the embodiments to affect drug release into plasma from Compound AM-2 administered orally.

Saline solutions of Compound AM-2 (which can be prepared as described in the examples herein) were dosed at 16 μmol/kg (6 mg/kg) with or without a co-dose of 55 μmol/kg (30 mg/kg) Compound 109 (Catalog No. 3081, Tocris Bioscience or Catalog No. WS38665, Waterstone Technology) as indicated in Table 7 via oral gavage into jugular vein-cannulated male Sprague Dawley rats (4 per group) that had been fasted for 16-18 hr prior to oral dosing. At specified time points, blood samples were drawn, harvested for plasma via centrifugation at 5,400 rpm at 4° C. for 5 min, and 100 μl plasma transferred from each sample into a fresh tube containing 2 μl of 50% formic acid. The tubes were vortexed for 5-10 seconds, immediately placed in dry ice and then stored in a −80° C. freezer until analysis by HPLC/MS.

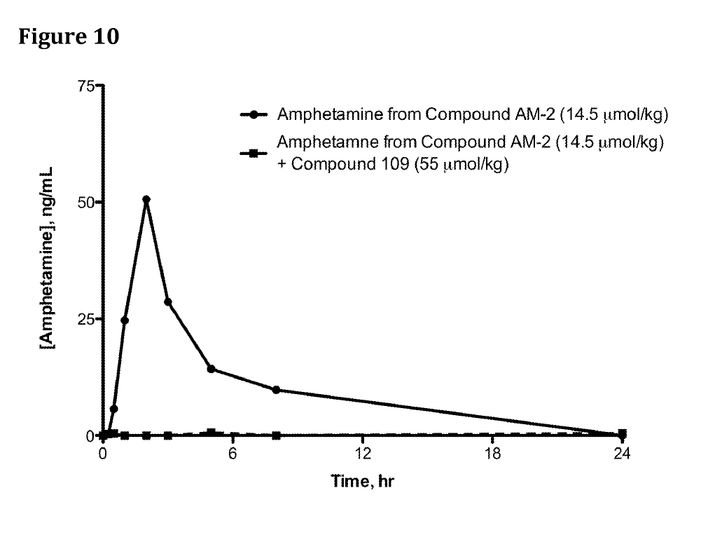

Table 10 and FIG. 10 provide amphetamine exposure results for rats administered with Compound AM-2 and with or without a co-dose of trypsin inhibitor. Results in Table 10 are reported, for each group of 4 rats, as (a) maximum plasma concentration (Cmax) of amphetamine (AMP) (average±standard deviation) and (b) time after administration of Compound AM-2, to reach maximum amphetamine concentration (Tmax) (average±standard deviation).

TABLE 10
Cmax and Tmax values of amphetamine in rat plasma

| | | Com- | | | |
|---|---|---|---|---|---|
| AM-2 | AM-2 | pound | Compound | AMP | Tmax ± |
| Dose, | Dose, | 109 Dose, | 109 Dose, | Cmax ± sd, | sd, |
| mg/kg | μmol/kg | mg/kg | μmol/kg | ng/mL | hr |
| 6 | 14.5 | 0 | 0 | 50.6 ± 6.3 | 2.0 ± 0.0 |
| 6 | 14.5 | 30 | 55 | 0.91 ± 1.1 | 12.3 ± 17 |

Lower limit of quantitation was 1.000 ng/mL.

FIG. 10 compares mean plasma concentrations over time of amphetamine release following PO administration of Compound AM-2 with or without a co-dose of trypsin inhibitor.

The results in Table 10 and FIG. 10 indicated Compound 109's ability to attenuate

Compound AM-2's ability to release amphetamine both by suppressing Cmax and by delaying Tmax.

### Example 23

### Pharmacokinetics of a GABA_{A} Inhibitor and a Hydromorphone Prodrug Following PO Administration to Dogs with or without Co-Administration of a Trypsin Inhibitor

This Example tests whether the pharmacokinetic profile of a GABA_{A} inhibitor is affected when administered with or without a prodrug of the embodiments and with or without a co-dose of trypsin inhibitor. This Example also tests whether the attenuation of hydromorphone release by a trypsin inhibitor is affected by the GABA_{A} inhibitor.

Purebred male young adult/adult beagles were fasted overnight. Each group of four dogs was administered prodrug Compound PC-5 (which can be prepared as described in Example 1 herein) and/or the GABA_{A} inhibitor Alprazolam XR (available from Mylan Pharmaceuticals, Inc, Morgantown, W. Va.), with or without trypsin inhibitor Compound 109 (available from Changzhou Institute of Materia Medica (China)), according to Table 11. Doses were administered in water via oral gavage for Compound PC-5 and Compound 109, and administered manually for Alprazolam XR tablets. The tablet dose was followed by approximately 10 mL of water to facilitate swallowing. Blood was collected from each animal via a jugular vein at various times over a 24-h period, centrifuged, and 0.8 mL plasma transferred to a fresh tube containing 8 μL formic acid; samples were vortexed, then immediately placed in dry ice, and stored in a −80° C. freezer until analysis by HPLC/MS.

Table 11 provides hydromorphone and Alprazolam XR exposure results for dogs administered the indicated compounds. Results in Table 11 are reported, for each group of dogs, as (a) maximum plasma concentration (Cmax) of hydromorphone (HM) (average±standard deviation), (b) time after administration of compound to reach maximum hydromorphone concentration (HM Tmax) (average±standard deviation), (c) maximum plasma concentration (Cmax) of Alprazolam XR (AZ) (average±standard deviation), and (d) time after administration of compound to reach maximum Alprazolam XR concentration (AZ Tmax) (average±standard deviation).

TABLE 11
Cmax and Tmax values of hydromorphone or Alprazolam XR in dog plasma

| Compound | Alprazolam | Compound | | | AZ Cmax ± | |
|---|---|---|---|---|---|---|
| PC-5 | XR | 109 | HM Cmax ± | HM Tmax ± | sd, | AZ Tmax ± |
| mg/kg | mg tablet | mg/kg | sd, ng/mL | sd, h | ng/mL | sd, h |
| 0.5# | n/a | n/a | 0.512 ± 0.17 | 2.00 ± 0.82 | n/a | n/a |
| 0.5# | 0.5* | n/a | 0.675 ± 0.24 | 1.50 ± 0.58 | 2.02 ± 0.46 | 4.00 ± 0.00 |
| 0.5# | 0.5§ | 1 | 0.0704 ± 0.050 | 1.25 ± 1.3 | 2.69 ± 2.7 | 2.25 ± 0.50 |
| n/a | 0.5* | n/a | n/a | n/a | 3.26 ± 2.3 | 2.75 ± 0.96 |
| n/a | 0.5§ | 1 | n/a | n/a | 2.89 ± 1.3 | 3.00 ± 0.82 |

*Lower limit of Alprazolam quantitation was 0.0500 ng/mL
§Lower limit of Alprazolam quantitation was 0.100 ng/mL
#Lower limit of hydromorphone quantitation was 0.0500 ng/mL
n/a = not applicable

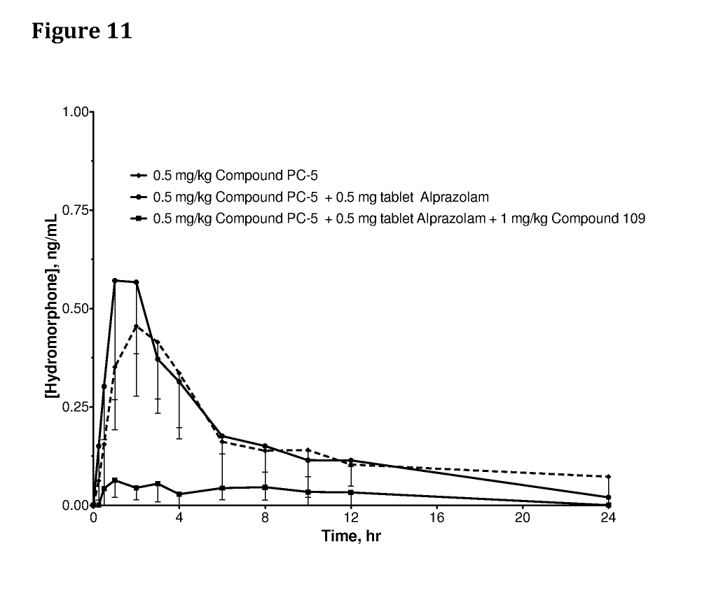

FIG. 11 compares mean plasma concentrations over time of hydromorphone following PO administration to dogs of (a) Compound PC-5, (b) co-administration of Compound PC-5 with Alprazolam XR, and (c) co-administration of Compound PC-5 and Compound 109 with Alprazolam XR.

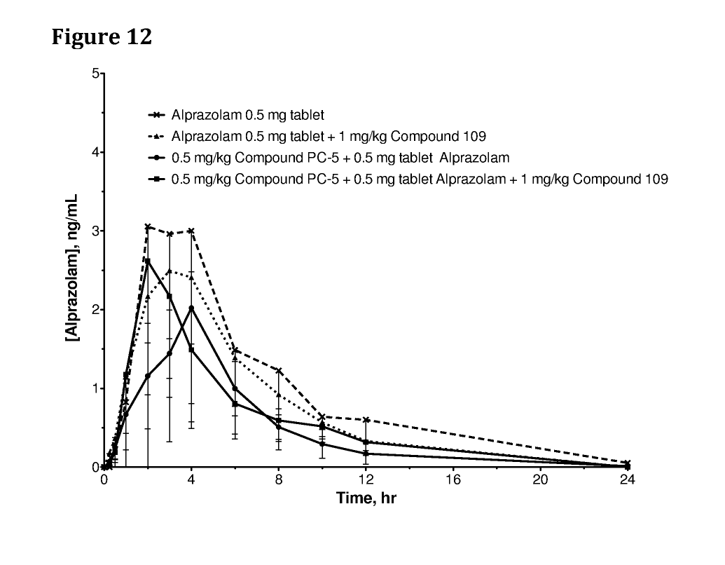

FIG. 12 compares mean plasma concentrations over time of Alprazolam XR following PO administration to dogs of (a) Alprazolam XR, (b) co-administration of Alprazolam XR with Compound 109, (c) co-administration of Alprazolam XR with Compound PC-5 and, (d) co-administration of Alprazolam XR with Compound PC-5 and Compound 109.

The results in Table 11 and FIG. 11 indicate that Alprazolam XR, administered orally to dogs with prodrug Compound PC-5, does not significantly affect hydromorphone release by Compound PC-5. Furthermore, Alprazolam XR, administered orally to dogs with prodrug Compound PC-5 and trypsin inhibitor Compound 109 does not significantly affect attenuation of hydromorphone release. The results in Table 11 and FIG. 12 indicate that the plasma levels of Alprazolam XR are not significantly affected by co-administration with (a) trypsin inhibitor Compound 109, (b) hydromorphone prodrug Compound PC-5, or (c) Compound 109 and Compound PC-5 administered together.

While the present invention has been described with reference to the specific embodiments thereof, it should be understood by those skilled in the art that various changes may be made and equivalents may be substituted without departing from the scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A composition comprising a GABA_{A} agonist and a GI enzyme inhibitor.

2. The composition of claim 1, wherein the GABA_{A} agonist is selected from benzodiazepines, non-benzodiazepines, barbiturates, neuroactive steroids, methaqualone, progabide, and tiagabine.

3. The composition of claim 1, wherein the GABA_{A} agonist is a benzodiazepine.

4. The composition of claim 1, wherein the GI enzyme inhibitor is a trypsin inhibitor.

5. The composition of claim 1, wherein the GI enzyme inhibitor is a lysine or arginine mimic.

6. The composition of claim 1, wherein the GI enzyme inhibitor is a compound of Formulae T-I to T-IV.

7. The composition of claim 1, wherein the GI enzyme inhibitor is Compound 109, which is 6-carbamimidoylnaphthalen-2-yl 4-(diaminomethyleneamino)benzoate.

8. A composition comprising (a) a GI enzyme inhibitor and (b) a first drug, wherein said first drug interacts in an additive or synergistic manner with a second drug to produce an adverse effect when the second drug is co-ingested as a GI enzyme-cleavable prodrug with the first drug.

9. The composition of claim 8, wherein the first drug is selected from a GABA_{A} agonist, a drug that interacts with an adrenergic receptor, an NMDA receptor antagonist, a monoamine oxidase inhibitor (MAOI), a central nervous system (CNS) depressant, and a drug that causes serotonin syndrome.

10. The composition of claim 9, wherein the first drug is a muscle relaxant.

11. A composition comprising a CNS depressant and a GI enzyme inhibitor.

12. The composition of claim 11, wherein the CNS depressant is GABA_{A} agonist.

13. The composition of claim 12, wherein the GABA_{A} agonist is selected from benzodiazepines, non-benzodiazepines, barbiturates, neuroactive steroids, methaqualone, progabide, and tiagabine.

14. The composition of claim 12, wherein the GABA_{A} agonist is a benzodiazepine.

15. The composition of claim 8, wherein the GI enzyme inhibitor is a trypsin inhibitor.

16. The composition of claim 8, wherein the GI enzyme inhibitor is a lysine or arginine mimic

17. The composition of claim 8, wherein the GI enzyme inhibitor is a compound of Formulae T-I to T-IV.

18. The composition of claim 8, wherein the GI enzyme inhibitor is Compound 109, which is 6-carbamimidoylnaphthalen-2-yl 4-(diaminomethyleneamino)benzoate.
